(19) United States
(12) Patent Application Publication
(10) Pub. No.: US 2003/0068650 A1
(43) Pub. Date: Apr. 10, 2003

(54) Target analysis for chemistry of specific and broad spectrum anti-infectives and other therapeutics

(76) Inventors: Jack Greenblatt, Toronto (CA); Aled Edwards, Toronto (CA); Cheryl Arrowsmith, North York (CA); John D. Mendlein, Encincitas, CA (US)

Correspondence Address: FOLEY HOAG LLP, PATENT GROUP, WORLD TRADE CENTER WEST, 155 SEAPORT BOULEVARD, BOSTON, MA, 02110-2600, US

(21) Appl. No.: 10/097,193

(22) Filed: Mar. 12, 2002

Related U.S. Application Data
(60) Provisional application No. 60/275,216, filed on Mar. 12, 2001.

(51) Int. Cl. C12Q 1/70; G01N 33/53

(52) U.S. Cl. 435/7.1; 435/5

(57) ABSTRACT

The present invention relates to methods for learning structural information about a molecule or molecular complex. The invention also provides methods for identifying a compound that binds to a molecule or molecular complex. The invention also provides methods for identifying a compound that binds to one molecule or molecular complex and not to one or more other molecules or molecular complexes. Other methods that are provided can be used to identify a compound that binds to at least two molecules or molecular complexes.

### RELATED APPLICATIONS

This application claims the benefit of U.S. Provisional Application No. 60/275,216, filed Mar. 12, 2001, which is incorporated herein in its entirety.

### INTRODUCTION

Discovery of novel drugs originally stemmed largely from empirical observations of the medicinal properties of various substances. For example, the observation that extracts of the foxglove plant alleviate congestive heart failure led to the discovery of digitalis and other cardiac glycosides present in foxglove. Alexander Fleming's observation that contamination of bacterial cultures by the mold Penicillium chrysogenum led to the discovery and isolation of the antibiotic penicillin. Once the structure of this prototype drug had been elucidated, chemical modification was undertaken to generate derivatives of the parent compound in an effort to find other drugs with similar activities, augmented potency, and/or diminished side effects. Such efforts stemming from penicillin led to discovery of, for example, methicillin.

Another technique for drug discovery is massive screening of candidate compounds for desired activity. For example, once the antibiotic effect of Penicillium chrysogenum had been discovered, thousands of other soil microorganisms were tested for their ability to kill bacteria. Such screening programs are often run using assays that model the disease state for which medical therapies are sought.

More recent efforts at drug design have relied on increasingly sophisticated methods for identifying desirable properties of candidate compounds and then synthesizing compounds on this basis. Such so-called “rational” drug design has resulted in more rapid lead identification because drugs put into the screening pipeline are already known to possess desirable properties. These properties may include affinity for various catalytic sites or regions in enzymes, other proteins, and nucleic acids, such as ATP binding sites, kinase domains, DNA binding sites, and other sites of protein-protein, protein-nucleic acid, and nucleic acid-nucleic acid interaction. The ability to interact at such sites may confer on a candidate compound the ability to abrogate, potentiate, or otherwise modulate the attendant interaction.

Anti-infective drugs are a particular goal of rational drug design due to the large and growing need to develop novel therapies directed against various infective organisms. Genomic sequence data, expression data, and proteomic data for such infective organisms provide a rich basis for identifying potential drug targets by rational drug design to modulate protein activity, or protein-protein, protein-nucleic acid, and nucleic acid-nucleic acid interactions necessary for a given microorganism to establish an infection or progress through its life cycle.

Past efforts at rational drug design have employed “structural” and “predictive” methods. Structural methods include mass spectroscopy (MS) nuclear magnetic resonance (NMR) and x-ray crystallography (XRC) characterization of proteins to determine structure information of a protein. Compounds may then be constructed using computer modeling which possess structural characteristics enabling them to access and interact with these sites, perhaps akin to designing a key to fit a particular lock. See Becker et al. (U.S. Pat. No. 5,834,228) for an example of using the structure of the apopain:Ac-DEVD-CHO complex as determined by x-ray crystallography to design drugs that inhibit apopain. Inouye et al. (U.S. Pat. No. 6,162,627) describe the use of NMR for structural analysis of a transmembrane sensor histidine kinase. Parekh et al. (U.S. Pat. No. 6,064,754) used MS to identify biomolecules in a biological sample. Balaji et al. (U.S. Pat. No. 5,579,250) employ computer modeling of conformational features of peptides for the purposes of rational drug discovery.

Individually, these techniques have been useful in promoting rational drug design. However, there remains in the art a need for highly selective structural characterization of potential drug targets and identification of compounds that may be useful as therapeutic agents. We have now discovered that combinations of mass spectroscopy, NMR and x-ray crystallography may be used to accelerate, enhance or enable interrogation of protein function or structure previously not available. These technologies may also be used to create high throughput platforms from rational drug design.

### SUMMARY OF THE INVENTION

The present invention provides novel methods for determining structure information of a polypeptide using two or more of the following techniques:

1) mass spectrometry to determine one or more properties of a protein, including primary sequence, post translation modification, protein-small molecule interaction, or protein-protein interaction ability;

2) NMR, including 1D NMR, multidimensional NMR, and multinuclear NMR, such as ¹⁵N/¹H HSQC spectra, to determine one or more properties of a protein including three dimensional structure, conformational states, aggregation level, state of protein folding or unfolding, or the dynamic properties of the protein; and

3) x-ray crystallography to determine one or more properties of a protein, including three dimensional structure, diffraction of its crystal form or its space group.

The invention also provides methods for determining structure information of a polypeptide in the presence and absence of another molecule, including other polypeptides, nucleic acids or small molecules, so as to aid in identifying druggable regions and designing therapeutically relevant compounds. The methods of the invention also provide means for designing, identifying or selecting small molecules that interact with a polypeptide and modulate its function or activity level. The methods of the invention also provide means to determine the selectivity of a molecule for interacting with, or modulating the activity of, two or more polypeptides.

In certain embodiments, the methods of the invention utilize functional assays to measure the activity of a polypeptide or to monitor the activity of a protein in the presence of one or more test compounds.

In another aspect, the methods of the invention may be used to identify inhibitors, agonists or antagonists against a target polypeptide, or biological complex, that may be used to treat any disease or other treatable condition of a patient (including humans and animals).

In other aspects, the information determined using the methods of the invention, such as sequence information about one or more polypeptides, and structural and functional information about the polypeptides, will be incorporated into databases. Such databases will provide investigators with a powerful tool to analyze the polypeptides and aid in the rapid discovery and design of therapeutic and diagnostic molecules.

The present invention further allows relationships between polypeptides for the same and multiple species to be compared by isolating and studying the various polypeptides using high throughput methods. By such comparison studies involving multi-variable analysis as appropriate, it is possible to identify drugs that will affect polypeptides from multiple species, or that will be selective for polypeptides from a particular species.

In other embodiments, the invention contemplates kits to carry out the methods of the invention including nucleic acids, polypeptides, crystallized polypeptides, antibodies, and other subject materials, and optionally instructions for their use. Uses for such kits include, for example, diagnostic and/or therapeutic applications.

The embodiments and practices of the present invention, other embodiments, and their features and characteristics, will be apparent from the description, figures and claims that follow, with all of the claims hereby being incorporated by this reference into this Summary.

### DETAILED DESCRIPTION OF THE INVENTION

1. Definitions

For convenience, certain terms employed in the specification, examples, and appended claims are collected here. Unless defined otherwise, all technical and scientific terms used herein have the same meaning as commonly understood by one of ordinary skill in the art to which this invention belongs. Generally, the nomenclature used herein and the laboratory procedures in spectroscopy, drug discovery, cell culture, molecular genetics, diagnostics, amino acid and nucleic acid chemistry described below are those well known and commonly employed in the art. The practice of the present invention will employ, unless otherwise indicated, conventional techniques of cell biology, cell culture, molecular biology, transgenic biology, microbiology, recombinant DNA, chemical syntheses, chemical analyses, biological assays, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press:1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Protein Purification: Principles and Practice, (R. K. Scopes, Third Edition, Springer Advanced Texts in Chemistry, 1994).

The articles “a” and “an” are used herein to refer to one or to more than one (i.e., to at least one) of the grammatical object of the article. By way of example, “an element” means one element or more than one element.

The term “amino acid” is intended to embrace all molecules, whether natural or synthetic, which include both an amino functionality and an acid functionality and capable of being included in a polymer of naturally occurring amino acids. Exemplary amino acids include naturally occurring amino acids; analogs, derivatives and congeners thereof; amino acid analogs having variant side chains; and all stereoisomers of any of any of the foregoing.

The term “agonist” as used herein, refers to a molecule which augments formation of a protein complex or which, when bound to a complex of the invention or a molecule in the complex, increases the amount of, or prolongs the duration of, the activity of the complex. Agonists may include proteins, nucleic acids, carbohydrates, or any other molecules, including, for example, chemicals, metals, organometallic agents, etc., that bind to a complex or molecule of the complex. Agonists also include a functional peptide or peptide fragment derived from a protein member of the subject complexes, or it may include a protein member itself. Peptide mimetics, synthetic molecules with physical structures designed to mimic structural features of particular peptides, may serve as agonists. The stimulation may be direct, or indirect, or by a competitive or non-competitive mechanism.

The term “animal” refers to mammals, including, for example, humans, primates, bovines, porcines, canines, felines, and rodents (such as mice and rats).

The term “antagonist”, as used herein, refers to a molecule which, when bound to a complex of the invention or a protein in the complex, decreases the amount of or duration of the activity of the complex or a protein member thereof, or decreases amount of complex formed. Antagonists may include proteins, including antibodies, that compete for binding at a binding region of a member of the complex, nucleic acids including anti-sense molecules that arrest expression of a member of the complex at the genetic level, carbohydrates, or any other molecules, including, for example, chemicals, metals, organo-metallic agents, etc., that bind to a mammalian, preferably human, protein, to an extent efficient for preventing complex formation or activity. Antagonists also include a peptide or peptide fragment derived from a protein, as well as dominant negative point mutations. Peptide mimetics, synthetic molecules with physical structures designed to mimic structural features of particular peptides, may serve as antagonists. The inhibition may be direct, or indirect, or by a competitive or non-competitive mechanism.

The terms “bait” or “bait protein” refer to a polypeptide which is used as a target to find other proteins which may associate with it. Typically, a bait protein is tagged or immobilized so as to allow easy isolation of complexes involving the bait protein.

The term “binding” refers to an association, which may be a stable association, between two molecules, e.g., between a polypeptide and a binding partner, due to, for example, electrostatic, hydrophobic, ionic and/or hydrogen-bond interactions under physiological conditions.

The term “binding pocket” refers to a region of a molecule or molecular complex, that, as a result of its shape, favorably associates with another chemical entity or modulator. Exemplary binding pockets include active sites, surface grooves or contours or surfaces of a protein or complex which are capable of participating in interactions with another modulator. Typically, the volume of which corresponds to a carbon based molecule of at least about 200 MW and often up to about 800 MW. Although in some stances of larger binding pockets it will be appreciated, particularly for binding pockets capable of binding natural products and open ring structures, the volume of such binding pockets may correspond to a carbon based molecule of at least about 600 MW and often up to about 1600 MW.

The terms “biological activity” or “bioactivity” or “activity” or “biological function” refer to an effector or antigenic function that is directly or indirectly performed by a polypeptide, nucleic acid, chemical entity, macromolecule, complex, species or the like (whether in its native, denatured or other conformation).

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. The term “recombinant cell” refers to a cell that contains heterologous nucleic acid, and the term “naturally occurring cell” refers to a cell that does not contain heterologous nucleic acid introduced by the hand of man.

A “comparison window,” as used herein, refers to a conceptual segment of at least 20 contiguous amino acid positions wherein a protein sequence may be compared to a reference sequence of at least 20 contiguous amino acids and wherein the portion of the protein sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Natl. Acad. Sci. (U.S.A.) 85:2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods may be identified.

The terms “compound”, “test compound” and “molecule” are used herein interchangeably and are meant to include, but are not limited to, peptides, nucleic acids, carbohydrates, small organic molecules, natural product extract libraries, and any other molecules (including, but not limited to, chemicals, metals and organometallic compounds).

The term “complex” refers to an association between at least two moieties (e.g. chemical or biochemical) that have an affinity for one another. Examples of complexes include associations between antigen/antibodies, lectin/avidin, target polynucleotide/probe oligonucleotide, antibody/anti-antibody, receptor/ligand, enzyme/ligand and the like. “Member of a complex” refers to one moiety of the complex, such as an antigen or ligand. “Protein complex” or “polypeptide complex” refers to a complex comprising at least one polypeptide.

A “compound with therapeutic activity” refers to a therapeutic compound that binds to a polypeptide to alter or modulate its function for a particular indication.

The term “conserved residue” refers to an amino acid that is a member of a group of amino acids having certain common properties. The term “conservative amino acid substitution” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). One example of a set of amino acid groups defined in this manner include:

(i) a charged group, consisting of Glu and Asp, Lys, Arg and His,

(ii) a positively-charged group, consisting of Lys, Arg and His,

(iii) a negatively-charged group, consisting of Glu and Asp,

(iv) an aromatic group, consisting of Phe, Tyr and Trp,

(v) a nitrogen ring group, consisting of His and Trp,

(vi) a large aliphatic nonpolar group, consisting of Val, Leu and Ile,

(vii) a slightly-polar group, consisting of Met and Cys,

(viii) a small-residue group, consisting of Ser, Thr, Asp, Asn, Gly, Ala, Glu, Gln and Pro,

(ix) an aliphatic group consisting of Val, Leu, Ile, Met and Cys, and

(x) a small hydroxyl group consisting of Ser and Thr.

The term “DNA sequence encoding a polypeptide” may refer to one or more genes, or an open reading frame thereof, within an organism. As is well known in the art, genes for a particular polypeptide may exist in single or multiple copies within the genome of an organism. Such duplicate genes may be identical or may have certain modifications, including nucleotide substitutions, additions or deletions, which all still code for polypeptides having substantially the same activity. Moreover, certain differences in nucleotide sequences may exist between individual organisms, which are called alleles. Such allelic differences may result in differences in amino acid sequence of the encoded polypeptide yet still encode a protein with the same or substantially similar biological activity.

The term “domain” as used herein refers to a region within a protein that comprises a particular structure or function different from that of other sections of the molecule.

The terms “druggable target,” “druggable region” and “druggable target region” are used herein interchangeably and refer to a region on the three dimensional structure of a polypeptide or complex which is a likely target for binding a modulator. A druggable region generally refers to a region wherein several amino acids of a polypeptide or complex would be capable of interacting with a modulator. Exemplary druggable regions including binding pockets, enzymatic active sites, surface grooves or contours or surfaces of a polypeptide or complex which are capable of participating in interactions with another molecule.

A “fusion protein” or “fusion polypeptide” refers to a polypeptide comprising a first amino acid sequence encoding a polypeptide linked to at least one other amino acid sequence encoding another polypeptide that is not substantially homologous with any domain of the first polypeptide. The two polypeptide sequences may be linked in frame. A fusion protein may include a domain which is found (albeit in a different protein) in an organism which also expresses the first protein, or it may be an “interspecies”, “intergenic”, etc. fusion expressed by different kinds of organisms. In various embodiments, the fusion polypeptide may comprise one or more amino acid sequences linked to the first polypeptide. In the case where more than one amino acid sequence is fused to the first polypeptide, the fusion sequences may be multiple copies of the same sequence, or alternatively, may be different amino acid sequences. The fusion polypeptides may be fused to the N-terminus, the C-terminus, or the N- and C-terminus of the first polypeptide. Exemplary fusion proteins include polypeptides comprising a glutathione S-transferase tag (GST-tag), histidine tag (His-tag), an immunoglobulin domain or an immunoglobulin binding domain.

As used herein, the term “gene” or “recombinant gene” refers to a nucleic acid comprising an open reading frame encoding a polypeptide of the present invention, including both exon and (optionally) intron sequences. A “recombinant gene” refers to nucleic acid encoding a polypeptide and comprising exon coding sequences, though it may optionally include intron sequences derived from a chromosomal gene. The term “intron” refers to a DNA sequence present in a given gene which is not translated into protein and is generally found between exons.

The term “having substantially similar biological activity” of a first molecule or complex, and like terms, refers to a biological activity of a first molecule or complex which is substantially similar to at least one of the biological activities of a second molecule or complex. A substantially similar biological activity means that the molecules or complexes carry out a similar function in the cell, e.g., a similar enzymatic reaction or a similar physiological process, etc. For example, two homologous proteins may have a substantially similar biological activity if they are involved in a similar enzymatic reaction, e.g., they are both kinases which catalyze phosphorylation of a substrate polypeptide, however, they may phosphorylate different regions on the same protein substrate or different substrate proteins altogether. Alternatively, two homologous proteins may also have a substantially similar biological activity if they are both involved in a similar physiological process, e.g., transcription. For example, two proteins may be transcription factors, however, they may bind to different DNA sequences or bind to different polypeptide interactors. Substantially similar biological activities may also be associated with proteins carrying out a similar structural role in the cell, for example, two membrane proteins.

The term “heavy-metal atoms” refers to an atom that can be used to solve an x-ray crystallography phase problem, including but not limited to a transition element, a lanthanide metal, or an actinide metal. Lanthanide metals include elements with atomic numbers between 57 and 71, inclusive. Actinide metals include elements with atomic numbers between 89 and 103, inclusive.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SLAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

The term “isolated”, as used herein with reference to proteins and protein complexes, refers to a preparation of protein or protein complex that is essentially free from contaminating proteins that normally would be present in association with the protein or complex, e.g., in the cellular milieu in which the protein or complex is found endogenously. Thus, an isolated protein complex is isolated from cellular components that normally would “contaminate” or interfere with the study of the complex in isolation, for instance while screening for modulators thereof. It is to be understood, however, that such an “isolated” complex may incorporate other proteins the modulation of which, by the subject protein or protein complex, is being investigated.

The term “isolated” as also used herein with respect to nucleic acids, such as DNA or RNA, refers to molecules separated from other DNAs, or RNAs, respectively, that are present in the natural source of the macromolecule. For example, isolated nucleic acids encoding a polypeptide preferably include no more than 10 kilobases (kb) of nucleic acid sequence which naturally immediately flanks a particular gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state.

The terms “label” or “labeled” refer to incorporation of a detectable marker into a molecule, such as a polypeptide. Various methods of labeling polypeptides are known in the art and may be used. Examples of labels for polypeptides include, but are not limited to, the following: radioisotopes, fluorescent labels, heavy atoms, enzymatic labels or reporter genes, chemiluminescent groups, biotinyl groups, predetermined polypeptide epitopes recognized by a secondary reporter (e.g., leucine zipper pair sequences, binding sites for secondary antibodies, metal binding domains, epitope tags). Examples and use of such labels are described in more detail below. In some embodiments, labels are attached by spacer arms of various lengths to reduce potential steric hindrance.

Polypeptides referred to herein as “mammalian homologs” of a protein refers to other mammalian paralogs, or other mammalian orthologs.

The term “modulation”, when used in reference to a functional property or biological activity or process (e.g., enzyme activity or receptor binding), refers to the capacity to either up regulate (e.g., activate or stimulate), down regulate (e.g., inhibit or suppress) or change a quality of such property, activity or process. In certain instances, such regulation may be contingent on the occurrence of a specific event, such as activation of a signal transduction pathway, and/or may be manifest only in particular cell types.

The term “modulator” refers to a polypeptide, nucleic acid, macromolecule, complex, molecule, small molecule, species or the like (naturally occurring or non-naturally occurring), or an extract made from biological materials such as bacteria, plants, fungi, or animal cells or tissues, that may be capable of causing modulation. Modulators may be evaluated for potential activity as inhibitors or activators (directly or indirectly) of a functional property, biological activity or process, or combination of them, (e.g., agonist, partial antagonist, partial agonist, inverse agonist, antagonist, anti-microbial agents, inhibitors of microbial infection or proliferation, and the like) by inclusion in assays. In such assays, many modulators may be screened at one time. The activity of a modulator may be known, unknown or partially known.

The term “motif” refers to an amino acid sequence that is commonly found in a protein of a particular structure or function. Typically a consensus sequence is defined to represent a particular motif. The consensus sequence need not be strictly defined and may contain positions of variability, degeneracy, variability of length, etc. The consensus sequence may be used to search a database to identify other proteins that may have a similar structure or function due to the presence of the motif in its amino acid sequence. For example, on-line databases may be searched with a consensus sequence in order to identify other proteins containing a particular motif. Various search algorithms and/or programs may be used, including FASTA, BLAST or ENTREZ. FASTA and BLAST are available as a part of the GCG sequence analysis package (University of Wisconsin, Madison, Wis.). ENTREZ is available through the National Center for Biotechnology Information, National Library of Medicine, National Institutes of Health, Bethesda, Md.

The term “naturally-occurring”, as applied to an object, refers to the fact that an object may be found in nature. For example, a polypeptide or polynucleotide sequence that is present in an organism (including bacteria) that may be isolated from a source in nature and which has not been intentionally modified by man in the laboratory is naturally-occurring.

The term “nucleic acid”, which is often used herein interchangeably with “polynucleotides”, refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The term “operably linked”, when describing the relationship between two nucleic acid regions, refers to a juxtaposition wherein the regions are in a relationship permitting them to function in their intended manner. For example, a control sequence “operably linked” to a coding sequence is ligated in such a way that expression of the coding sequence is achieved under conditions compatible with the control sequences, such as when the appropriate molecules (e.g., inducers and polymerases) are bound to the control or regulatory sequence(s).

The terms “pharmaceutical agent” or “drug” refer to a compound or composition capable of inducing a desired therapeutic effect when properly administered to a patient.

The term “phenotype” refers to the entire physical, biochemical, and physiological makeup of a cell, e.g., having any one trait or any group of traits.

The term “polypeptide”, and the terms “protein” and “peptide” which are used interchangeably herein, refers to a polymer of amino acids. Exemplary polypeptides include gene products, naturally occurring proteins, homologs, orthologs, paralogs, fragments, and other equivalents and analogs of the foregoing.

The term “polypeptide fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions may occur at the amino-terminus or carboxy-terminus of the reference polypeptide. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long.

The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “substantially free of other contaminating proteins”) is defined as encompassing individual preparations of each of the component proteins comprising less than 20% (by dry weight) contaminating protein, and preferably comprises less than 5% contaminating protein. Functional forms of each of the component proteins can be prepared as purified preparations by using a cloned gene as described in the attached examples. By “purified”, it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% to 90% by dry weight, more preferably in the range of 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above.

“Isolated” and “purified” do not encompass either protein in its native state (e.g. as a part of a cell), or as part of a cell lysate, or that have been separated into components (e.g., in an acrylamide gel) but not obtained either as pure (e.g. lacking contaminating proteins) substances or solutions. The term isolated as used herein also refers to a component protein that is substantially free of cellular material or culture medium when produced by recombinant DNA techniques, or chemical precursors or other chemicals when chemically synthesized.

The term “recombinant protein” refers to a protein which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein, is meant to include within the meaning of “recombinant protein” those proteins having an amino acid sequence of a native protein, or an amino acid sequence similar thereto which is generated by mutations including substitutions and deletions of a naturally occurring protein.

The term “regulatory sequence” is a generic term used throughout the specification to refer to polynucleotide sequences, such as initiation signals, enhancers, and promoters, that are necessary or desirable to effect the expression of coding and non-coding sequences to which they are operably linked. Exemplary regulatory sequences are described in Goeddel; Gene Expression Technology: Methods in Enzymology, Academic Press, San Diego, Calif. (1990), and include, for example, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. The nature and use of such control sequences may differ depending upon the host organism. In prokaryotes, such regulatory sequences generally include promoter, ribosomal binding site, and transcription termination sequences. The term “regulatory sequence” is intended to include, at a minimum, components whose presence may influence expression, and may also include additional components whose presence is advantageous, for example, leader sequences and fusion partner sequences. In certain embodiments, transcription of a polynucleotide sequence is under the control of a promoter sequence (or other regulatory sequence) which controls the expression of the polynucleotide in a cell-type in which expression is intended. It will also be understood that the polynucleotide can be under the control of regulatory sequences which are the same or different from those sequences which control expression of the naturally-occurring form of the polynucleotide.

As used herein, a “reporter gene construct” is a nucleic acid that includes a “reporter gene” operatively linked to a transcriptional regulatory sequence. Transcription of the reporter gene is controlled by these sequences. The transcriptional regulatory sequences can include a promoter and other regulatory regions, such as enhancer sequences, that modulate the level of expression of a reporter gene in response to the level of a substrate protein. Examples of such reporter genes include, but are not limited to, luciferase, fluorescent protein (e.g., green fluorescent protein), chloramphenicol acetyl transferase, ss-galactosidase, secreted placental alkaline phosphatase, ss-lactamase, human growth hormone, and other secreted enzyme reporters. Generally, a reporter gene encodes a polypeptide not otherwise produced by the host cell, which is detectable by analysis of the cell(s), e.g., by the direct fluorometric, radioisotopic or spectrophotometric analysis of the cell(s) and preferably without the need to kill the cells for signal analysis. In certain instances, a reporter gene encodes an enzyme, which produces a change in fluorometric properties of the host cell, which is detectable by qualitative, quantitative or semiquantitative function or transcriptional activation. Exemplary enzymes include esterases, β-lactamase, phosphatases, peroxidases, proteases (tissue plasminogen activator or urokinase) and other enzymes whose function may be detected by appropriate chromogenic or fluorogenic substrates known to those skilled in the art or developed in the future.

By “semi-purified”, with respect to protein preparations, it is meant that the proteins have been previously separated from other cellular or viral proteins. For instance, in contrast to whole cell lysates, the proteins of reconstituted conjugation system, together with the substrate protein, can be present in the mixture to at least 50% purity relative to all other proteins in the mixture, more preferably are present at least 75% purity, and even more preferably are present at 90-95% purity.

The term “semi-purified cell extract” or, alternatively, “fractionated lysate”, as used herein, refers to a cell lysate which has been treated so as to substantially remove at least one component of the whole cell lysate, or to substantially enrich at least one component of the whole cell lysate. “Substantially remove”, as used herein, means to remove at least 10%, more preferably at least 50%, and still more preferably at least 80%, of the component of the whole cell lysate. “Substantially enrich”, as used herein, means to enrich by at least 10%, more preferably by at least 30%, and still more preferably at least about 50%, at least one component of the whole cell lysate compared to another component of the whole cell lysate. The term “semi-purified cell extract” is also intended to include the lysate from a cell, when the cell has been treated so as to have substantially more, or substantially less, of a given component than a control cell. For example, a cell which has been modified (by, e.g., recombinant DNA techniques) to produce none (or very little) of a particular cellular component, will, upon cell lysis, yield a semi-purified cell extract.

The term “sequence homology” refers to the proportion of base matches between two nucleic acid sequences or the proportion of amino acid matches between two amino acid sequences. When sequence homology is expressed as a percentage, e.g., 50%, the percentage denotes the proportion of matches over the length of sequence from a desired sequence (e.g., SEQ. ID NO. 1) that is compared to some other sequence. Gaps (in either of the two sequences) are permitted to maximize matching; gap lengths of 15 bases or less are usually used, 6 bases or less are used more frequently, with 2 bases or less used even more frequently. The term “sequence identity” means that sequences are identical (i.e., on a nucleotide-by-nucleotide basis for nucleic acids or amino acid-by-amino acid basis for polypeptides) over a window of comparison. The term “percentage of sequence identity” is calculated by comparing two optimally aligned sequences over the window of comparison, determining the number of positions at which the identical amino acids occurs in both sequences to yield the number of matched positions, dividing the number of matched positions by the total number of positions in the window of comparison (i.e., the window size), and multiplying the result by 100 to yield the percentage of sequence identity. Methods to calculate sequence identity are known to those of skill in the art and described in further detail below.

The terms “signal transduction,” “signaling,” “signal transduction pathway,” “signaling pathway,” etc. are used herein interchangeably and refer to the processing of physical or chemical signals from the cellular environment through the cell membrane, and may occur through one or more of several mechanisms, such as activation/inactivation of enzymes (such as proteases, or other enzymes which may alter phosphorylation patterns or other post-translational modifications), activation of ion channels or intracellular ion stores, effector enzyme activation via guanine nucleotide binding protein intermediates, formation of inositol phosphate, activation or inactivation of adenylyl cyclase, direct activation (or inhibition) of a transcriptional factor and/or activation, etc.

The term “small molecule” refers to a compound, which has a molecular weight of less than about 5 kD, preferably less than about 2.5 kD, more preferably less than about 1.5 kD, and most preferably less than about 0.9 kD. Small molecules may be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention. The term “small organic molecule” refers to a small molecule that is often identified as being an organic or medicinal compound, and does not include molecules that are exclusively nucleic acids, peptides or polypeptides.

The term “soluble” as used herein with reference to a polypeptide, means that upon expression in cell culture, at least some portion of the polypeptide expressed remains in the cytoplasmic fraction of the cell and does not fractionate with the cellular debris upon lysis and centrifugation of the lysate. Solubility of a polypeptide may be increased by a variety of art recognized methods, including fusion to a heterologous amino acid sequence, deletion of amino acid residues, amino acid substitution (e.g., enriching the sequence with amino acid residues having hydrophilic side chains), and chemical modification (e.g., addition of hydrophilic groups). The solubility of polypeptides may be measured using a variety of art recognized techniques, including, dynamic light scattering to determine aggregation state, UV absorption, centrifugation to separate aggregated from non-aggregated material, and SDS gel electrophoresis (e.g., the amount of protein in the soluble fraction is compared to the amount of protein in the soluble and insoluble fractions combined). When expressed in a host cell, polypeptides may be at least about 1%, 2%, 5%, 10%, 20%, 30%, 40%, 50%, 60%, 70%, 80%, 90% or more soluble, e.g., at least about 1%, 2%, 5%, 10%, 20%, 30%, 40%, 50%, 60%, 70%, 80%, 90% or more of the total amount of protein expressed in the cell is found in the cytoplasmic fraction. In certain embodiments, a one liter culture of cells expressing a polypeptide will produce at least about 0. 1, 0.2, 0.5, 1, 2, 5, 10, 20, 30, 40, 50 milligrams or more of soluble protein. In an exemplary embodiment, a polypeptide is at least about 10% soluble and will produce at least about 1 milligram of protein from a one liter cell culture.

As used herein, the term “specifically hybridizes” refers to the ability of a nucleic acid probe/primer of the invention to hybridize to at least 15, 25, 50 or 100 consecutive nucleotides of a target gene sequence, or a sequence complementary thereto, or naturally occurring mutants thereof, such that it has less than 15%, preferably less than 10%, and more preferably less than 5% background hybridization to a cellular nucleic acid (e.g., MRNA or genomic DNA) other than the target gene.

The term “structurally stable domain” refers to a portion of a polypeptide which is suitable for structural characterization by NMR and/or x-ray crystallography.

The term “structural motif” of a polypeptide or protein refers to a structural motif of a polypeptide or protein that, although it may have different amino acid sequences, may result in a similar structure, wherein by structure is meant that the motif forms generally the same tertiary structure, or that certain amino acid residues within the motif, or alternatively their backbone or side chains (which may or may not include the Ca) are positioned in a like relationship with respect to one another in the motif. Such structural motifs are known to be important to the functionality observed for proteins.

As used herein, the term “structural coordinates” refers to a set of values that define the position of one or more amino acid residues with reference to a system of axes. The term refers to a data set that defines the three dimensional structure of a molecule or molecules (e.g. Cartesian coordinates, temperature factors, and occupancies). Structural coordinates can be slightly modified and still render nearly identical three dimensional structures. A measure of a unique set of structural coordinates is the root-mean-square deviation of the resulting structure. Structural coordinates that render three dimensional structures (in particular a three dimensional structure of a ligand binding pocket) that deviate from one another by a root-mean-square deviation of less than 5 Å, 4 Å, 3 Å, 2 Å, or 1.5 Å may be viewed by a person of ordinary skill in the art as very similar.

As applied to polypeptides, “substantial sequence identity” means that two mammalian peptide sequences, when optimally aligned, such as by the programs GAP or BESTFIT using default gap which share at least 90 percent sequence identity, preferably at least 95 percent sequence identity, more preferably at least 99 percent sequence identity or more. Preferably, residue positions which are not identical differ by conservative amino acid substitutions. For example, the substitution of amino acids having similar chemical properties such as charge or polarity are not likely to effect the properties of a protein. Examples include glutamine for asparagine or glutamic acid for aspartic acid.

The term “target” refers to a biochemical entity involved in a biological process and against which a targeted molecule or construct is directed. In certain instances, a target may be a tumor, a site of infection, a molecular structure to which a targeting moiety is directed (e.g., a hapten, epitope, receptor, macromolecule, etc.), or a type of tissue. In many instances, targets are proteins that play a useful role in the physiology or biology of an organism.

As used herein, the term “test compound” means any compound which is potentially capable of associating with a protein, and/or inhibiting or enhancing its enzymatic acitivity or its ability to interact with another molecule. The test compound may be designed or obtained from a library of compounds which may comprise peptides, as well as other compounds, such as small organic molecules and particularly new lead compounds. By way of example, the test compound may be a natural substance, a biological macromolecule, or an extract made from biological materials such as bacteria, fungi, or animal (particularly mammalian) cells or tissues, an organic or an inorganic molecule, a synthetic test compound, a semi-synthetic test compound, a carbohydrate, a monosaccharide, an oligosaccharide or polysaccharide, a glycolipid, a glycopeptide, a saponin, a heterocyclic compound, a structural or functional mimetic, a peptide, a peptidomimetic, a derivatised test compound, a peptide cleaved from a whole protein, or a peptides synthesised synthetically (such as, by way of example, either using a peptide synthesizer or by recombinant techniques or combinations thereof), a recombinant test compound, a natural or a non-natural test compound, a fusion protein or equivalent thereof and mutants, derivatives or combinations thereof.

As used herein, the term “transfection” means the introduction of a nucleic acid, e.g., an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of a polypeptide of the present invention or where anti-sense expression occurs from the transferred gene so that the expression of a naturally-occurring form of the gene is disrupted.

As used herein, the term “transgene” means a nucleic acid sequence, which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, such as introns, that may be necessary for optimal expression of a selected nucleic acid.

The term “transgenic animal” refers to any animal, for example, a mouse, rat or other non-human mammal, a bird or an amphibian, in which one or more of the cells of the animal contain heterologous nucleic acid introduced by way of human intervention, such as by transgenic techniques well known in the art. The nucleic acid is introduced into the cell, directly or indirectly, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of a protein. However, transgenic animals in which the recombinant gene is silent are also contemplated.

The term “treating” is intended to encompass curing as well as ameliorating at least one symptom of a condition or disease.

The term “unit cell” refers to the smallest and simplest volume element (i.e. parallelpiped-shaped block) of a crystal that is completely representative of the unit of pattern of the crystal. The unit cell axial lengths are represented by a, b, and c. Those of skill in the art understand that a set of atomic coordinates determined by X-ray crystallography is not without standard error.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto. The term “whole lysate” refers to a cell lysate which has not been manipulated, e.g. either fractionated, depleted or charged, beyond the step of merely lysing the cell to form the lysate.

2. Methods of the Invention

In one embodiment, the invention provides a method for identifying a site or binding region on a protein, wherein the site has a particular structure that is not present in one or more other proteins. A “site” or a “binding region” is a region in a biological molecule, e.g., a protein, to which a molecule is capable of binding with a certain affinity, e.g., e.g., 10⁻⁶ M; 10⁻⁷ M; 10⁻¹ M or 10⁻⁹ M. A site can be within a structurally stable domain. The method may comprise (i) providing isolated and purified first and second proteins; (ii) subjecting a portion of the purified and isolated first and second proteins to MS; (iii) subjecting a portion of the purified and isolated first and second proteins to NMR spectroscopic analysis; and (iv) subjecting a portion of the purified and isolated first and second proteins to X-ray diffraction. Alternatively, 1, 2 or 3 of these steps may be sufficient. The method may then comprise analyzing the structural information obtained to identify one or more sites (or binding regions) on the first or second protein that are not present on the second and first protein, respectively. Preferably, the method will use proteins from the same sample or preparation but this is not generally necessary. This method, e.g., allows the identification of sites on the proteins that have a sufficiently different structure that one would not expect a drug binding to the first or second protein to bind to the second or first protein, respectively. Thus, this method permits to design drugs that act selectively on one protein. The method may be used, e.g., for identifying drugs that kill specifically an infectious agent without significantly affecting the subject, e.g., a human, being treated for elimination of the infectious agent. The method may also be used to identify a drug that will act specifically or selectively on a particular protein in a cell of a subject, but essentially not on other proteins, thereby permitting the identification of drugs that have reduced toxicity.

This method may be used to identify a drug that will bind to an modulate the activity of a class of proteins of one type, such as viral proteins, and not eukaryotic proteins, to give a drug that is a broad spectrum antiviral.

In another embodiment, the invention provides a method for identifying a site or binding region on a protein, wherein the site has a particular structure that is present with sufficient similarity in one or more other proteins. The particular structure may be at least 1, 2, 3, 4, 5, 7 or 10 amino acids that are either linked together or not. Generally, a particular structure refers to the structure of a region in a protein to which another molecule can bind with significant affinity, e.g., 10⁻⁶ M; 10⁻⁷ M; 10⁻⁸ M or 10⁻⁹ M. The method may involve the same steps (i) to (iv) as the method in the previous paragraph, or at least 1, 2, or 3 steps thereof. The method may then comprise analyzing the structural information obtained to identify one or more sites or binding regions on the first or second protein that are present with sufficient similarity in the second or first protein, respectively. This method allows the identification of sites or binding regions on the proteins that have a sufficiently similar structure that one would expect a drug binding to the first or second protein to bind to the second or first protein, respectively. This method can be used, e.g., for identifying drugs that act on several different proteins, such as several different proteins of a pathogenic organism, and thereby increase the efficiency of the drug. The method can also be used, e.g., for identifying drugs that act on several different proteins in the cells of a subject, wherein the several different proteins are involved in a particular disease.

In yet other embodiments, the invention is a combination of the two above-described methods. For example, in another embodiment, the invention provides a method for identifying a site on a protein, wherein the site has a particular structure that is present with sufficient similarity in a first set of one or more other proteins and is essentially not present in a second set of one or more proteins.

The site on a protein can be any region to which one expects that a molecule would be able to bind and optionally modulate the activity of the protein. Exemplary sites include binding pockets, active sites, and sites to which cofactors or other molecules bind. Other sites include those, which when bound by a molecule trigger a conformational change of the protein, thereby potentially affecting the activity of the protein or binding of other molecules to it.

In another embodiment, the invention provides a method for identifying a compound that binds preferably to a first protein or complex relative to a second protein or complex. In a preferred embodiment, the method comprises subjecting the first and the second protein or complex to analysis by mass spectrometric (MS) analysis to obtain structural information on the first and the second protein. The method preferably further comprises subjecting the protein or complex to NMR spectroscopic analysis and/or X-ray diffraction in the presence and/or absence of a test compound. Analysis in the presence and in the absence of a test compound indicates the location at which the test compound binds to the protein or complex, since different results will be obtained in NMR analysis of a protein and a protein to which a ligand is binding. Similarly, X-ray diffraction will indicate whether a compound binds and if so, where the compound binds.

Accordingly, in one embodiment, the invention provides a method for identifying a compound that binds preferably to a first protein relative to a second protein, comprising (i) providing isolated and purified first and second proteins; (ii) subjecting a portion of the purified and isolated first and second proteins to MS; (iii) subjecting a portion of the purified and isolated first and second proteins to NMR spectroscopic analysis in the presence of a test compound; (iv) subjecting a portion of the purified and isolated first and second proteins to NMR spectroscopic analysis in the absence of a test compound; (v) subjecting a portion of the purified and isolated first and second proteins to X-ray diffraction in the presence of a test compound; (vi) subjecting a portion of the purified and isolated first and second proteins to X-ray diffraction in the absence of a test compound; to thereby determine whether the test compound binds to the two proteins, and if so, to determine the location in the first and second proteins to which the test compound binds. In other embodiments, only some of these steps are performed, e.g., at least two, 3, 4, or 5 of the above-steps are performed.

The method is applicable to identifying a compound that binds preferably or selectively to a first protein or complex relative to at least two other proteins or complexes. The method is also applicable to identifying a compound that binds to at least two proteins or complexes. The number of proteins or complexes that can be analyzed, e.g., in parallel, can be at least 3, 5, 7, 10 or more. In a preferred embodiment, the first and the other at least two proteins or complexes are subjected to MS and to NMR spectroscopic analysis and/or X-ray diffraction. In certain embodiments, some proteins or complexes are not subjected to MS or NMR or X-ray diffraction.

In another embodiment, two or more test compounds are tested simultaneously in the same sample. For example, two or more compounds can be incubated with the protein or complex or portion thereof in NMR and/or X-ray crystallography. The results will indicate whether one or more of the test compounds bind to a site on the protein or complex.

A person of skill in the art will recognize that the method described herein can also be performed on a molecular complex, e.g., a protein complex. Thus, the invention provides methods for identifying sites of a molecular complex, e.g., a protein complex, having a particular structure, that is similar or different to those found on one or more other proteins or molecular complexes. The invention also provides methods for identifying compounds or drugs that bind to one or more molecular complexes and which essentially do not bind to one or more other molecular complexes or proteins.

A person of skill in the art will also recognize that, when referring to proteins or protein complexes, the proteins can be modified, e.g., with posttranslation modifications, such as glycosylation, pegylation, phosphorylation. It will also be apparent that molecules other than proteins can be used according to the methods of the invention.

In some embodiments, the method comprises obtaining MS, NMR and/or X-ray information on a protein or complex and comparing the information to data on one or more other proteins or complexes that are present in a computer readable storage medium. The comparison of the structural information obtained can be conducted with a computer.

It can be the same protein or complex that is subjected to these different analyses or different portions of the protein or complex can be used in the different analyses. For example, MS analysis can be conducted on the full length protein and NMR and/or x-ray analysis conducted on a portion of the protein. The portion can be selected, e.g., based on the results obtained from the MS. For example if the MS results indicate the presence of a domain in a particular region, the NMR and/or x-ray analysis can be conducted on the particular domain, or on a region that does not include the domain.

The proteins or complexes that can be analyzed according to the methods of the invention can be soluble or membrane bound proteins or complexes. They can be extracellular, membraneous, or intracellular, e.g., cytoplasmic or nuclear proteins or complexes. The proteins or complexes can be prokaryotic or eukaryotic, e.g., vertebrate, such as mammalian, e.g., human, simian, equine, bovine, ovine, porcine, canine, feline, or rodent proteins. Proteins can also be viral or from plants.

Exemplary proteins can be targeted include growth or differentiation factors; hormones; lymphokines; interleukins (ILs); tumor necrosis factor (TNF); lymphotoxins; soluble or membrane receptors to ligands, e.g., receptors to growth or differentiation factors; protiens from the transcription machinery, e.g., RNA polymerase; transcription factors; proteins that mediate signal transduction in a cell; proteins encoded by oncogenes; cell surface proteins; enzymes; and structural proteins. Table I provides examples of proteins that can be used in the invention, as well as diseases with which these proteins are associated.

Prokaryotic proteins that can be targeted, particularly of pathogenic microorganisms include cell wall proteins; capsule proteins; ribosomes; proteins from the transcription machinery, e.g., RNA polymerase; transcription factors; nucleic acid binding proteins; and other cytoplasmic proteins.

Viral proteins that can be targeted include coat proteins; proteins necessary for transcription, such as reverse transcriptase; glycoprotein; nucleocapsid protein; and matrix protein. Exemplary viruses include retroviruses, such as lentiviruses, e.g., human immunodeficiency virus (HIV); hepatitis viruses; papillomaviruses, herpesviruses; and viruses from the following families: papovaviruses, adenoviruses, poxviruses, parvoviruses, picomaviruses, orthomyoxoviruses, paramyxoviruses, reoviruses, togaviruses and falviviruses, bunayaviridae, and rhabdoviruses.

In one embodiment of the invention, the method comprises analyzing two or more proteins or complexes that are from different species, e.g., one being human and the other being from yeast. This allows the identification of druggable sites and drugs that are specific to one species, e.g., which kill cells of one species but not of others. In an illustrative embodiment, the structures of the two or more proteins or complexes from different species are compared to identify potential drug binding sites that are present in the protein or complex of one species but not in the protein or complex of the others, such that the drug would only have an effect on the protein or complex of the species having a protein to which the compound binds.

The proteins that may be used in the invention may be significantly related, e.g., they may have an amino acid sequence that is at least about 60%; 70%; 80%; 90% or 95% identical or homologous to each other. The proteins can also be structurally similar, i.e., having a three dimensional structure that has similar features, even if their amino acid sequence is not similar. The methods described herein are particularly suitable to identify sites for drug targeting or compounds that bind to such sites in family of genes, at least since the x-ray diffraction information (i.e., coordinates) obtained from one protein may be used to determine the coordinates of a related protein, e.g., by molecular replacement. The methods of the invention can also be used to compare two proteins having similar structural motifs, e.g., DNA binding domain; transcriptional activation domain; active site; dimerization or multimerization domains; and domains interacting with specific molecules; e.g., other proteins.

Other proteins that may be used include a wild-type and a mutated protein, e.g., a protein whose mutated form is associated with a disease. The methods of the invention permit the identification of compounds that can selectively interact with the mutated form, thereby preventing its biological activity, and its deleterious effect on a subject expressing such mutant protein.

The proteins to be analyzed can be from the same gene family. Thus, a compound that binds to and potentially modulates the biological activity of at least two proteins from a same gene family can be identified according to the methods of the invention. It is desirable to identify drugs that interact with several proteins in one family to obtain a stronger effect. For example, where one desires to inhibit the activity of a protein that belongs to a family of proteins, it may be desirable to also inhibit the activity of other proteins from that family, to prevent other family members to take over the biological activity that the first protein carried out in a cell. Alternatively, in certain cases, it may be desirable to specifically target one member of a family and not the others.

Exemplary gene families include kinases; phosphatases; nuclear receptors and phosphodiestereases, as further described in Table 1.

In another embodiment, the invention provides a method for identifying a compound that binds to a protein or complex. The method can comprise (i) providing an isolated and purified protein; (ii) subjecting a portion of the isolated and purified protein to MS; (iii) subjecting a portion of the isolated and purified protein to NMR spectroscopic analysis in the presence of a test compound; (iv) subjecting a portion of the isolated and purified protein to NMR spectroscopic analysis in the absence of a test compound; (v) subjecting a portion of the isolated and purified protein to X-ray diffraction in the presence of a test compound; and/or (vi) subjecting a portion of the isolated and purified protein to X-ray diffraction in the absence of a test compound; to thereby determine whether the test compound binds to the protein, and if so, to determine the location in the protein to which the test compound binds. In other embodiments, only some of these steps are performed, e.g., at least two, 3, 4, or 5 of the above-steps are performed. For example, the method may include MS, and NMR in the presence and absence of the test compound. Another method may include MS, and X-ray diffraction in the presence or absence of the test compound. Yet another method may include MS, and NMR and X-ray diffraction in the presence of the test compound.

In yet another embodiment, the invention provides methods for obtaining structural information about one or more proteins. The structural information can be the three dimensional structure of at least a portion of a protein or complex. For example, structural information can be information on the secondary (folding into helices and sheets), tertiary (folding between helices and sheets and combination of secondary features into compact shapes, e.g., domains), or quaternary structure (organization of several polypeptide chains into a single protein molecule) of at least a portion of a protein or complex. The method may comprise (i) providing an isolated and purified protein; (ii) subjecting a portion of the isolated and purified protein to MS; (iii) subjecting a portion of the isolated and purified protein to NMR spectroscopic analysis in the presence of a test compound; (iv) subjecting a portion of the isolated and purified protein to NMR spectroscopic analysis in the absence of a test compound; (v) subjecting a portion of the isolated and purified protein to X-ray diffraction in the presence of a test compound; and/or (vi) subjecting a portion of the isolated and purified protein to X-ray diffraction in the absence of a test compound, to thereby obtain structural information. The structural information may contain coordinates of at least a region of the protein, which may be used, e.g., in rational drug design to identify potential compounds that interact with the protein.

The methods described herein may be combined with rational drug design. For example, in methods for identifying a site of a protein, wherein the site has a particular structure, after having identified a site of interest, the method may further include identifying a drug by rational drug design. In an illustrative embodiment, a method described herein further comprises a computer-assisted method, comprising: (a) supplying a computer modeling application with a set of structure coordinates, and optionally structural information from MS and/or NMR, of a protein or complex; (b) supplying the computer modeling application with a set of structure coordinates of a chemical entity; and determining whether the chemical entity is expected to bind to the protein or complex. The structure coordinates and optionally other structural information may be those of a portion of the protein including the site of interest. A site of interest, e.g., a binding pocket, may be defined by sets of points having a root mean square deviation of less than from about 1.5 Å to about 1.1 Å from points representing the backbone atoms of the amino acids of the site of interest. Alternatively, a site of interest may also be defined by sets of points having a root mean square deviation of less than about 1.5 Å or 1.1 Å from points representing the side chain atoms and optionally the Ca atoms of the amino acids of the site of interest. Determining whether the chemical entity binds to the site of interest of a protein and thereby potentially acts as a modulator can comprise performing a fitting operation between the chemical entity and the site of interest of the protein or molecular complex, followed by computationally analyzing the results of the fitting operation to quantify the association between the chemical entity and the site of interest. The method can further comprise screening a library of chemical entities.

A rational drug design step can also be performed as follows: (a) supplying a computer modeling application with the structural coordinates and/or other structural information of a particular site on a protein or complex; (b) supplying the computer modeling application with a set of structure coordinates for a chemical entity; (c) evaluating the potential binding interactions between the chemical entity and the site of interest of the protein or molecular complex; (d) structurally modifying the chemical entity to yield a set of structure coordinates for a modified chemical entity; and (e) determining whether the modified chemical entity binds to the site of interest and optionally modulates the activity of the protein or complex. The set of structure coordinates for the chemical entity can be obtained from a chemical fragment library.

In another embodiment, rational drug design comprises a computer-assisted method for designing a compound that binds to the site of interest de novo comprising, e.g., (a) supplying a computer modeling application with a set of structure coordinates and optionally other structural information of the site of interest; (b) computationally building a chemical entity represented by set of structure coordinates; and (c) determining whether the chemical entity binds the site of interest. The method may then further comprise supplying or synthesizing the compound, then assaying it to determine whether it binds and whether it modulates the activity of the protein or complex.

The invention also provides a method for making a compound that binds to a site of interest on a protein or complex, the method comprising synthesizing a chemical entity to yield a compound, the chemical entity having been identified by any of the methods described herein.

The invention also provides methods for identifying a site on a protein, wherein the site has a particular structure that is present or absent from other proteins, or methods for identifying a compound that binds to one or more proteins, wherein the method comprises subjecting the protein to MS to identify a particular domain or portion of the protein, e.g., a structurally stable domain, and then subjecting that particular domain or portion of the protein to NMR and/or X-ray diffraction in the presence or absence of the compound.

The particular steps of methods described herein do not have to be performed in a particular order. For example, NMR or X-ray diffraction analysis can be conducted prior to MS analysis. In certain embodiments, the steps are conducted essentially simultaneously.

In yet other embodiments, the same protein sample is used for one or more of the steps. For example, a protein sample can be subjected to NMR and then directly introduced into the mass spectrometer for MS analysis.

In other embodiments of the methods disclosed herein, the proteins or complexes are labeled. For example, the proteins can be labeled with one or more labels. Labels can be heavy atom labels for X-ray crystallography and labels used in NMR analysis. In an illustrative embodiment, a fraction of a purified protein is subjected to MS; another fraction of the purified protein is labeled with a heavy atom; and yet a third fraction is labeled with a label suitable for NMR analysis. Alternatively, one and the same protein sample can be labeled with a heavy atom and with a label suitable for NMR analysis.

In embodiments using a test compound, the test compound can be also be labeled, e.g., with a heavy atom and/or with a label suitable for NMR analysis, or other labels such as those described herein.

The methods of the invention can also be combined with one or more activity assay, e.g., biological assays for determining whether the compound that was identified is a modulator of the biological activity of the protein or complex. Such assays can be conducted as further described herein.

In another embodiment, the site or binding region of the protein is accessible to the exterior, i.e., located on the outside of a protein.

In yet another embodiment, the method comprises determining a first binding region or structurally stable domain from a first target, e.g., a protein, using one or more of the following MS, NMR or x-ray crystallography; (b) determining a second binding region or structurally stable domain from a second target, e.g., protein, using one or more of the following MS, NMR or x-ray crystallography. The method may further comprise comparing the first structurally stable domain to said second structurally stable domain to identify specific coordinating groups that face the outside of the protein that have comparable physical properties in 3 dimensions. The first target may be from a first species and said second target may be from a second species. Determining of said first structurally stable domain may comprise a MS determination and the first species may be a bacterial species. Determining of the first structurally stable domain may comprises an x-ray determination at a resolution of 2.5 Angstroms or better and the first species is a rodent species. Determining of the structural domain may comprise incubating the first or second structural domain with a small molecule ligand that coordinates with specific coordinating groups. Comparing may comprise identifying specific coordinating groups with a small molecule ligand. Specific coordinating groups may also be identified in a second and third protein or structurally stable domain thereof. The first target may be incubated with at least about 5 small molecule ligands that share a common substructure comprising at least one or more of the following: 6 carbons, 2 fluorines and two ring structures. The targets may have from about 30% to about 90%; from about 60% to about 90%; or from about 80% to about 909% homology or identity at the amino acid sequence level. In certain embodiments, the first and second target were not previously known to bind a common ligand, e.g., as it occurs in nature or a synthetic or recombinant entity. The targets can be from different bacterial species. The targets can also be from different rodent species. At least one of the targets can be recombinantly expressed, e.g., in bacteria.

In another embodiment, the invention provides a method of identifying a binding region on a target, comprising (a) determining a first binding site or structurally stable domain from a first target using one or more of the following MS, NMR or x-ray crystallography; (b) determining a first affinity site for a chemical entity in said first structurally stable domain; (c) determining a first undesired site for said chemical entity in said first structurally stable domain, and (d) modifying said chemical entity to have less binding energy at said undesired site. The term “affinity site” refers to a site or binding region on a biological molecule that is present in several biological molecules. For example, an ATP binding site in a kinase is referred to herein as an “affinity site.” An “undesired site” refers to a site in a biological molecule, e.g., a protein, which, when it interacts with a chemical property of a molecule, e.g., a chemical entity, results in undesirable, e.g., toxic, effects on the cell and a subject when administered to a subject. The method may further include determining a first selectivity site for said chemical entity. The term “selectivity site” refers to the site or binding region of a biological molecule that may not be found on other biological molecules. An exemplary selectivity site is a catalytic domain of a kinase. In certain instances, a single of compound may bind to the same affinity site across a number of proteins that have a substantially similar biological function, whereas the same or different compounds may only bind one of the selectivity sites for such proteins.

Binding to an affinity site or other site may reduce the binding energy or provide binding energy by at least about 20%; 30%; 50% or 60%. Determining of the affinity site may comprise determining the costructure of said modified form. Determining of the first selectivity site may provide for determining at least a one third log less binding between an apparent Kd of said chemical entity between first structural domain and a second structural domain. Determining of the first selectivity site may include determining the co-structure of the modified form of the chemical entity with said first structural domain. Determining of the first selectivity site provides for determining at least a one third log more binding between an apparent Kd of the chemical entity between the first structural domain and a second structural domain; wherein the first structural domain and the second structural domain have more than 60% homology at the amino acid sequence level.

In another embodiment, determining the first undesired site comprises determining at least about a one third log decrease in activity between an apparent P450 activity of the chemical entity between the structural domain and a second structurally stable domain. Determining of an apparent P450 activity can be with cells. Determining the first undesired property site may comprise determining at least about 20% less activity between an apparent P450 activity of the chemical entity and a modified form of the chemical entity that binds to the first undesired site with less binding energy. Determining an apparent P450 activity may include a determination of affinity of both the chemical entity and the modified form for a P450. Determining the first undesired site may further comprise comparing said chemical entity bound to the first structural domain and bound to a second structural domain from a second target. The first and the second structural domain may be from a kinase, a phosphodiesterase, or a protease. The first structural domain may be from a micro-organism and the second structural domain may be from a human.

In yet another embodiment, the first undesired property site may interact with a chemical property of a chemical entity that leads to an increase in apparent P450 activity of the chemical entity compared to a modified form of said chemical entity that binds to the first undesired site with less binding energy. The first undesired site may interact with a chemical property of the chemical entity that leads to a decrease in an apparent mammalian membrane permeability of said chemical entity compared to a modified form of said chemical entity that binds to the first undesired property site with less binding energy. The first undesired site may interact with a chemical property of said chemical entity that leads to an increase in an apparent mammalian toxicity of said chemical entity compared to a modified form of the chemical entity that binds to the first undesired property site with less binding energy. The first undesired site may interact with a chemical property of the chemical entity that leads to an increase in an apparent mammalian excretion of the chemical entity compared to a modified form of the chemical entity that binds to the first undesired property site with less binding energy. The first undesired site may also interact with a chemical property of said chemical entity that leads to an increase in an apparent mammalian blood brain transport of said chemical entity compared to a modified form of the chemical entity that binds to the first undesired property site with less binding energy. The modified form may have has less amino acid transporter activity with one or more amino acid transport systems.

3. Polypeptides

The methods of the present invention utilize polypeptides, or fragments thereof, suitable for structural characterization by various techniques, including, for example, mass spectroscopy, NMR and x-ray crystallography. In certain embodiments, the polypeptides are soluble, purified and/or isolated polypeptides which may optionally comprise a tag or label to facilitate expression, purification and/or structural or functional characterization.

In certain embodiments, a polypeptide which may be used in accordance with the methods of the invention is a fusion protein containing a domain which increases it solubility and/or facilitates its purification, identification, detection, and/or structural or functional characterization. Exemplary domains, include, for example, glutathione S-transferase (GST), protein A, protein G, calmodulin-binding peptide, thioredoxin, maltose binding protein, HA, myc, poly arginine, poly His, poly His-Asp or FLAG fusion proteins and tags. Additional exemplary domains include domains that alter protein localization in vivo, such as signal peptides, type III secretion system-targeting peptides, transcytosis domains, nuclear localization signals, etc. In various embodiments, a polypeptide may comprise one or more heterologous fusions. Polypeptides may contain multiple copies of the same fusion domain or may contain fusions to two or more different domains. The fusions may occur at the N-terminus of the polypeptide, at the C-terminus of the polypeptide, or at both the N- and C-terminus of the polypeptide. It is also within the scope of the invention to include linker sequences between the polypeptide and the fusion domain in order to facilitate construction of the fusion protein or to optimize protein expression or structural constraints of the fusion protein. In another embodiment, the polypeptide may be constructed so as to contain protease cleavage sites between the fusion polypeptide and polypeptide in order to remove the tag after protein expression or thereafter. Examples of suitable endoproteases, include, for example, Factor Xa and TEV proteases.

In another embodiment, a polypeptide which may be used in accordance with the methods of the invention may be modified so that its rate of traversing the cellular membrane is increased. For example, the polypeptide may be fused to a second peptide which promotes “transcytosis,” e.g., uptake of the peptide by cells. The peptide may be a portion of the HIV transactivator (TAT) protein, such as the fragment corresponding to residues 37-62 or 48-60 of TAT, portions which have been observed to be rapidly taken up by a cell in vitro (Green and Loewenstein, (1989) Cell 55:1179-1188). Alternatively, the internalizing peptide may be derived from the Drosophila antennapedia protein, or homologs thereof. The 60 amino acid long homeodomain of the homeo-protein antennapedia has been demonstrated to translocate through biological membranes and can facilitate the translocation of heterologous polypeptides to which it is couples. Thus, polypeptides may be fused to a peptide consisting of about amino acids 42-58 of Drosophila antennapedia or shorter fragments for transcytosis (Derossi et al. (1996) J Biol Chem 271:18188-18193; Derossi et al. (1994) J Biol Chem 269:10444-10450; and Perez et al. (1992) J Cell Sci 102:717-722). The transcytosis polypeptide may also be a non-naturally occurring membrane-translocating sequence (MTS), such as the peptide sequences disclosed in U.S. Pat. No. 6,248,558.

In another embodiment, a polypeptide which may be used in accordance with the methods of the invention is labeled with an isotopic label to facilitate its detection and or structural characterization using nuclear magnetic resonance or another applicable technique. Exemplary isotopic labels include radioisotopic labels such as, for example, potassium-40 (⁴K), carbon-14 (¹⁴C), tritium (³H), sulphur-35 (³⁵S), phosphorus-32 (³²p), technetium-99m (^{99m}Tc), thallium-201 (201Tl), gallium-67 (⁶⁷Ga), indium-111 (¹¹¹In), iodine-123 (¹²³I), iodine-131 (¹³¹I), yttrium-90 (⁹⁰Y), samarium-153 (¹⁵³Sm), rhenium-186 (¹⁸⁶Re), rhenium-188 (¹⁸⁸Re), dysprosium-165 (¹⁶⁵Dy) and holmium-166 (166Ho). The isotopic label may also be an atom with non zero nuclear spin, including, for example, hydrogen-1 (¹H), hydrogen-2 (²H), hydrogen-3 (³H), phosphorous-31 (³¹p), sodium-23 (²³Na), nitrogen-14 (¹⁴N), nitrogen-15 (¹⁵N), carbon-13 (¹³C) and fluorine-19 (¹⁹F). In certain embodiments, the polypeptide is uniformly labeled with an isotopic label, for example, wherein at least 50%, 70%, 80%, 90%, 95%, or 98% of the possible labels in the polypeptide are labeled, e.g., wherein at least 50%, 70%, 80%, 90%, 95%, or 98% of the nitrogen atoms in the polypeptide are 1⁵N, and/or wherein at least 50%, 70%, 80%, 90%, 95%, or 98% of the carbon atoms in the polypeptide are ¹³C, and/or wherein at least 50%, 70%, 80%, 90%, 95%, or 98% of the hydrogen atoms in the polypeptide are ²H. In other embodiments, the isotopic label is located in one or more specific locations within the polypeptide, for example, the label may be specifically incorporated into one or more of the leucine residues of the polypeptide. The invention also encompasses the embodiment wherein a single polypeptide comprises two or more different isotopic labels, for example, the polypeptide comprises both ¹⁵N and ¹³C labeling.

In yet another embodiment, the polypeptides which may be used in accordance with the methods of the invention are labeled to facilitate structural characterization using x-ray crystallography or another applicable technique. Exemplary labels include heavy atom labels such as, for example, cobalt, selenium, krypton, bromine, strontium, molybdenum, ruthenium, rhodium, palladium, silver, cadmium, tin, iodine, xenon, barium, lanthanum, cerium, praseodymium, neodymium, samarium, europium, gadolinium, terbium, dysprosium, holmium, erbium, thulium, ytterbium, lutetium, tantalum, tungsten, rhenium, osmium, iridium, platinum, gold, mercury, thallium, lead, thorium and uranium. In an exemplary embodiment, the polypeptides are labeled with seleno-methionine.

In another embodiment, the polypeptides which may be used in accordance with the methods of the invention comprise two or more labels in a single polypeptide so as to facilitate structural characterization of a single preparation of the polypeptide using different structural techniques. For example, a single polypeptide may contain one or more labels suitable for structural characterization by NMR (e.g., one or more isotopic labels) and one or more labels suitable for characterization by x-ray crystallography (e.g., one or more heavy atom labels). In an exemplary embodiment, the polypeptide is labeled with ¹⁵N, ¹³C and seleno-methionine.

In still another embodiment, the polypeptides which may be used in accordance with the methods of the invention are labeled with a fluorescent label to facilitate their detection, purification, or structural characterization. In an exemplary embodiment, a polypeptide is fused to a heterologous polypeptide sequence which produces a detectable fluorescent signal, including, for example, green fluorescent protein (GFP), enhanced green fluorescent protein (EGFP), Renilla Reniformis green fluorescent protein, GFPmut2, GFPuv4, enhanced yellow fluorescent protein (EYFP), enhanced cyan fluorescent protein (ECFP), enhanced blue fluorescent protein (EBFP), citrine and red fluorescent protein from discosoma (dsRED).

In other embodiments, the polypeptides which may be used in accordance with the methods of the invention are contained within a vessels useful for manipulation of the polypeptide sample. For example, the polypeptides may be contained within a microtiter plate to facilitate detection, proteolytic digestion, screening or purification of the polypeptide. The polypeptides may also be contained within an NMR tube in order to enable characterization by nuclear magnetic resonance techniques.

In still other embodiments, the polypeptides which may be used in accordance with the methods of the invention are crystallized and mounted for examination by x-ray crystallography as described further below.

In certain embodiments, it may be advantageous to provide naturally-occurring or experimentally derived homologs of a polypeptide used in accordance with the methods of the invention. Such homologs may function in a limited capacity as a modulator to promote or inhibit a subset of the biological activities of the naturally-occurring form of the polypeptide. For instance, antagonistic homologs may be generated which interfere with the ability of the wild-type polypeptide to associate with certain proteins, but which do not substantially interfere with the formation of complexes between the native polypeptide and other cellular proteins.

In certain embodiments, it may be advantageous to utilize fragments derived from full length proteins. Isolated peptidyl portions of proteins may be obtained by screening polypeptides recombinantly produced from the corresponding fragment of the nucleic acid encoding such polypeptides. In addition, fragments may be chemically synthesized using techniques known in the art such as conventional Merrifield solid phase f-Moc or t-Boc chemistry. For example, proteins may be arbitrarily divided into fragments of desired length with no overlap of the fragments, or may be divided into overlapping fragments of a desired length. The fragments may be produced (recombinantly or by chemical synthesis) and tested to identify those peptidyl fragments having a desired property, for example, the capability of functioning as a modulator of the polypeptides. In an illustrative embodiment, peptidyl portions of a protein of the invention may be tested for binding activity, as well as inhibitory ability, by expression as, for example, thioredoxin fusion proteins, each of which contains a discrete fragment of a protein of the invention (see, for example, U.S. Pat. Nos. 5,270,181 and 5,292,646; and PCT publication WO 94/02502).

In other embodiments, it may be useful to modify the structure of a polypeptide so as to enhance its stability and facilitate use in the methods of the invention. Such modified polypeptides, when designed to retain at least one activity of the naturally-occurring form of the protein, are considered “functional equivalents” of the un-modified polypeptide. Such modified polypeptides may be produced, for instance, by amino acid substitution, deletion, or addition, which substitutions may consist in whole or part by conservative amino acid substitutions.

For instance, it is reasonable to expect that an isolated conservative amino acid substitution, such as replacement of a leucine with an isoleucine or valine, an aspartate with a glutamate, a threonine with a serine, will not have a major effect on the biological activity of the resulting molecule. Whether a change in the amino acid sequence of a polypeptide results in a functional homolog may be readily determined by assessing the ability of the variant polypeptide to produce a response similar to that of the wild-type protein. Polypeptides in which more than one replacement has taken place may readily be tested in the same manner.

In other embodiments, a polypeptide which may be used in accordance with the methods of the invention may be part of a library of polypeptides. Such libraries may contain polypeptides having a common characteristic, such as, for example, a common species of origin, a substantially similar functionally activity, orthologs of a protein from a variety of species, proteins in a particular biosynthetic pathway, proteins derived from a particular organelle, etc. In exemplary embodiments, the polypeptides may be part of library derived from a non-membrane proteins from specific cell type, membrane-associated proteins from a particular cell type, proteins in a specific organelle (e.g. nucleus, ER, Golgi, ribosome or mitochondria), or proteins in a pathway (e.g. Ca pathway, CRE, NFAT, Jac Stat, etc.).

In other embodiments, polypeptides which may be used in accordance with the methods of the invention, include kinases, proteases, phosphatases, P450s, conjugation enzymes, ATPases, GTPase, nucleotide binding proteins, DNA processing enzymes, helicases, polymerases, RNA polymerases, DNA polymerases, GPCRs, intracellular receptors, metabolic enzymes, nuclear receptors, channels, phosphodiesterases, essential bacterial proteins, Ca binding proteins, bacterial proteins, non-membrane bacterial proteins, human proteins that bind viral proteins, viral proteins, and nonmembrane viral proteins. In exemplary embodiments, the polypeptides which are used in accordance with the methods of the invention are bacterial proteins derived from Eschericia coli, Helicobacter pylori, Pseudomonas aeruginosa, Chlaydia trachomatis, Haemophilus influenzae, Neisseria meningitidis, Rickettsia prowazekii, Borrelia burgdorferi, Bacillus subtilis, Staphylococcus aureus, Streptococcus pneumoniae, Mycoplasma genitalium, or Enterococcus faecalis.

This invention further contemplates a method of generating sets of combinatorial mutants of polypeptides, as well as truncation mutants, and is especially useful for identifying potential variant sequences (e.g. homologs). The purpose of screening such combinatorial libraries is to generate, for example, homologs which may modulate the activity of a polypeptide, or alternatively, which possess novel activities all together. Combinatorially-derived homologs may be generated which have a selective potency relative to a naturally occurring protein. Such homologs may be used in the development of therapeutics.

Likewise, mutagenesis may give rise to homologs which have intracellular half-lives dramatically different than the corresponding wild-type protein. For example, the altered protein may be rendered either more stable or less stable to proteolytic degradation or other cellular process which result in destruction of, or otherwise inactivation of the protein. Such homologs, and the genes which encode them, may be utilized to alter protein expression by modulating the half-life of the protein. As above, such proteins may be used for the development of therapeutics or treatment.

In similar fashion protein homologs may be generated by the present combinatorial approach to act as antagonists, in that they are able to interfere with the activity of the corresponding wild-type protein.

In a representative embodiment of this method, the amino acid sequences for a population of protein homologs are aligned, preferably to promote the highest homology possible. Such a population of variants may include, for example, homologs from one or more species, or homologs from the same species but which differ due to mutation. Amino acids which appear at each position of the aligned sequences are selected to create a degenerate set of combinatorial sequences. In certain embodiments, the combinatorial library is produced by way of a degenerate library of genes encoding a library of polypeptides which each include at least a portion of potential protein sequences. For instance, a mixture of synthetic oligonucleotides may be enzymatically ligated into gene sequences such that the degenerate set of potential nucleotide sequences are expressible as individual polypeptides, or alternatively, as a set of larger fusion proteins (e.g. for phage display).

There are many ways by which the library of potential homologs may be generated from a degenerate oligonucleotide sequence. Chemical synthesis of a degenerate gene sequence may be carried out in an automatic DNA synthesizer, and the synthetic genes may then be ligated into an appropriate vector for expression. One purpose of a degenerate set of genes is to provide, in one mixture, all of the sequences encoding the desired set of potential protein sequences. The synthesis of degenerate oligonucleotides is well known in the art (see for example, Narang, SA (1983) Tetrahedron 39:3; Itakura et al., (1981) Recombinant DNA, Proc. 3rd Cleveland Sympos. Macromolecules, ed. AG Walton, Amsterdam: Elsevier pp. 273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis may be utilized to generate a combinatorial library. For example, protein homologs (both agonist and antagonist forms) may be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of proteins.

A wide range of techniques are known in the art for screening gene products of combinatorial libraries made by point mutations and truncations, and for screening cDNA libraries for gene products having a certain property. Such techniques will be generally adaptable for rapid screening of the gene libraries generated by the combinatorial mutagenesis of protein homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In an illustrative embodiment of a screening assay, candidate combinatorial gene products are displayed on the surface of a cell and the ability of particular cells or viral particles to bind to the combinatorial gene product is detected in a “panning assay”. For instance, the gene library may be cloned into the gene for a surface membrane protein of a bacterial cell (Ladner et al., WO 88/06630; Fuchs et al., (1991) Bio/Technology 9:1370-1371; and Goward et al., (1992) TIBS 18:136-140), and the resulting fusion protein detected by panning, e.g. using a fluorescently labeled molecule which binds the cell surface protein, e.g. FITC-substrate, to score for potentially functional homologs. Cells may be visually inspected and separated under a fluorescence microscope, or, when the morphology of the cell permits, separated by a fluorescence-activated cell sorter. This method may be used to identify substrates or other polypeptides that can interact with a polypeptide.

In similar fashion, the gene library may be expressed as a fusion protein on the surface of a viral particle. For instance, in the filamentous phage system, foreign peptide sequences may be expressed on the surface of infectious phage, thereby conferring two benefits. First, because these phage may be applied to affinity matrices at very high concentrations, a large number of phage may be screened at one time. Second, because each infectious phage displays the combinatorial gene product on its surface, if a particular phage is recovered from an affinity matrix in low yield, the phage may be amplified by another round of infection. The group of alnost identical E. coli filamentous phages M13, fd, and fl are most often used in phage display libraries, as either of the phage gIII or gVIII coat proteins may be used to generate fusion proteins without disrupting the ultimate packaging of the viral particle (Ladner et al., PCT publication WO 90/02909; Garrard et al., PCT publication WO 92/09690; Marks et al., (1992) J. Biol. Chem. 267:16007-16010; Griffiths et al., (1993) EMBO J. 12:725-734; Clackson et al., (1991) Nature 352:624-628; and Barbas et al., (1992) PNAS USA 89:4457-4461). Other phage coat proteins may be used as appropriate.

The invention also provides for reduction of the subject proteins to generate mimetics, e.g. peptide or non-peptide agents, which are able to mimic binding of the authentic protein to another cellular partner. Such mutagenic techniques as described above, as well as the thioredoxin system, are also particularly useful for mapping the determinants of a protein which participates in a protein-protein interaction with another protein. To illustrate, the critical residues of a protein which are involved in molecular recognition of a substrate protein may be determined and used to generate peptidomimetics that may bind to the substrate protein. The peptidomimetic may then be used as an inhibitor of the wild-type protein by binding to the substrate and covering up the critical residues needed for interaction with the wild-type protein, thereby preventing interaction of the protein and the substrate. By employing, for example, scanning mutagenesis to map the amino acid residues of a protein which are involved in binding a substrate polypeptide, peptidomimetic compounds may be generated which mimic those residues in binding to the substrate. For instance, non-hydrolyzable peptide analogs of such residues may be generated using benzodiazepine (e.g., see Freidinger et al., in Peptides: Chemistry and Biology, G.R. Marshall ed., ESCOM Publisher: Leiden, Netherlands, 1988), azepine (e.g., see Huffinan et al., in Peptides: Chemistry and Biology, G.R. Marshall ed., ESCOM Publisher: Leiden, Netherlands, 1988), substituted gama lactam rings (Garvey et al., in Peptides: Chemistry and Biology, G.R. Marshall ed., ESCOM Publisher: Leiden, Netherlands, 1988), keto-methylene pseudopeptides (Ewenson et al., (1986) J. Med. Chem. 29:295; and Ewenson et al., in Peptides: Structure and Function (Proceedings of the 9th American Peptide Symposium) Pierce Chemical Co. Rockland, Ill., 1985), β-turn dipeptide cores (Nagai et al., (1985) Tetrahedron Lett 26:647; and Sato et al., (1986) J Chem Soc Perkin Trans 1:1231), and β-aminoalcohols (Gordon et al., (1985) Biochem Biophys Res Commun 126:419; and Dann et al., (1986) Biochem Biophys Res Commun 134:71).

The present invention further pertains to methods of producing the polypeptides which may be used in accordance with the methods of the invention. For example, a host cell transfected with an expression vector encoding a polypeptide may be cultured under appropriate conditions to allow expression of the polypeptide to occur. The polypeptide may be secreted and isolated from a mixture of cells and medium containing the polypeptide. Alternatively, the polypeptide may be retained cytoplasmically and the cells harvested, lysed and the protein isolated. A cell culture includes host cells, media and other byproducts. Suitable media for cell culture are well known in the art. The polypeptide may be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of a polypeptide.

Thus, a nucleotide sequence derived from the cloning of a gene encoding all or a selected portion of polypeptide, may be used to produce a recombinant form of the protein via microbial or eukaryotic cellular processes. Ligating the gene sequence into a polynucleotide construct, such as an expression vector, and transforming or transfecting into hosts, either eukaryotic (yeast, avian, insect or mammalian) or prokaryotic (bacterial cells), are standard procedures. Similar procedures, or modifications thereof, may be employed to prepare recombinant polypeptides by microbial means or tissue-culture technology in accord with the subject invention.

Expression vehicles for production of a recombinant protein include plasmids and other vectors. For instance, suitable vectors for the expression of a polypeptide include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al., (1983) in Experimental Manipulation of Gene Expression, ed. M. Inouye Academic Press, p. 83). These vectors may replicate in E. coli due the presence of the pBR322 ori, and in S. cerevisiae due to the replication determinant of the yeast 2 micron plasmid. In addition, drug resistance markers such as ampicillin may be used.

In certain embodiments, mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant protein by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In another variation, protein production may be achieved using in vitro translation systems. In vitro translation systems are, generally, a translation system which is a cell-free extract containing at least the minimum elements necessary for translation of an RNA molecule into a protein. An in vitro translation system typically comprises at least ribosomes, tRNAs, initiator methionyl-tRNAMet, proteins or complexes involved in translation, e.g., eIF2, eIF3, the cap-binding (CB) complex, comprising the cap-binding protein (CBP) and eukaryotic initiation factor 4F (eIF4F). A variety of in vitro translation systems are well known in the art and include commercially available kits. Examples of in vitro translation systems include eukaryotic lysates, such as rabbit reticulocyte lysates, rabbit oocyte lysates, human cell lysates, insect cell lysates and wheat germ extracts. Lysates are commercially available from manufacturers such as Promega Corp., Madison, Wis.; Stratagene, La Jolla, Calif.; Amersham, Arlington Heights, Ill.; and GIBCO/BRL, Grand Island, N.Y. In vitro translation systems typically comprise macromolecules, such as enzymes, translation, initiation and elongation factors, chemical reagents, and ribosomes. In addition, an in vitro transcription system may be used. Such systems typically comprise at least an RNA polymerase holoenzyme, ribonucleotides and any necessary transcription initiation, elongation and termination factors. In vitro transcription and translation may be coupled in a “one pot” reaction to produce proteins from one or more isolated DNAs.

When expression of a carboxy terminal fragment of a polypeptide is desired, i.e. a truncation mutant, it may be necessary to add a start codon (ATG) to the oligonucleotide fragment containing the desired sequence to be expressed. It is well known in the art that a methionine at the N-terminal position may be enzymatically cleaved by the use of the enzyme methionine aminopeptidase (MAP). MAP has been cloned from E. coli (Ben-Bassat et al., (1987) J. Bacteriol. 169:751-757) and Salmonella typhimurium and its in vitro activity has been demonstrated on recombinant proteins (Miller et al., (1987) PNAS USA 84:2718-1722). Therefore, removal of an N-terminal methionine, if desired, may be achieved either in vivo by expressing such recombinant polypeptides in a host which produces MAP (e.g., E. coli or CM89 or S. cerevisiae), or in vitro by use of purified MAP (e.g., procedure of Miller et al.).

Alternatively, coding sequences for a polypeptide of interest may be incorporated as a part of a fusion gene including a nucleotide sequence encoding a different polypeptide. This type of expression system can be useful under conditions where it is desirable, e.g., to produce an immunogenic fragment of a polypeptide. For example, the VP6 capsid protein of rotavirus may be used as an immunologic cartier protein for portions of polypeptide, either in the monomeric form or in the form of a viral particle. The nucleic acid sequences corresponding to the portion of a polypeptide to which antibodies are to be raised may be incorporated into a fusion gene construct which includes coding sequences for a late vaccinia virus structural protein to produce a set of recombinant viruses expressing fusion proteins comprising a portion of the protein as part of the virion. The Hepatitis B surface antigen may also be utilized in this role as well. Similarly, chimeric constructs coding for fusion proteins containing a portion of a polypeptide and the poliovirus capsid protein may be created to enhance immunogenicity (see, for example, EP Publication NO: 0259149; and Evans et al., (1989) Nature 339:385; Huang et al., (1988) J. ViroL 62:3855; and Schlienger et al., (1992) J. Virol. 66:2).

In another embodiment, a fusion gene coding for a purification leader sequence, such as a poly-(His)/enterokinase cleavage site sequence at the N-terminus of the desired portion of the recombinant protein, may allow purification of the expressed fusion protein by affinity chromatography using a Ni²⁺ metal resin. The purification leader sequence may then be subsequently removed by treatment with enterokinase to provide the purified protein (e.g., see Hochuli et al., (1987) J. Chromatography 411: 177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene may be synthesized by conventional techniques including automated DNA synthesizers. Alternatively, PCR amplification of gene fragments may be carried out using anchor primers which give rise to complementary overhangs between two consecutive gene fragments which may subsequently be annealed to generate a chimeric gene sequence (see, for example, Current Protocols in Molecular Biology, eds. Ausubel et al., John Wiley & Sons: 1992).

In certain embodiments, the polypeptides which may be used in accordance with the methods of the invention may be synthesized chemically, ribosomally in a cell free system, or ribosomally within a cell. Chemical synthesis of polypeptides may be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. Native chemical ligation employs a chemoselective reaction of two unprotected peptide segments to produce a transient thioester-linked intermediate. The transient thioester-linked intermediate then spontaneously undergoes a rearrangement to provide the full length ligation product having a native peptide bond at the ligation site. Full length ligation products are chemically identical to proteins produced by cell free synthesis. Full length ligation products may be refolded and/or oxidized, as allowed, to form native disulfide-containing protein molecules. (see e.g., U.S. Pat. Nos. 6,184,344 and 6,174,530; and T. W. Muir et al., Curr. Opin. Biotech. (1993): vol. 4, p 420; M. Miller, et al., Science (1989): vol. 246, p 1149; A. Wlodawer, et al., Science (1989): vol. 245, p 616; L. H. Huang, et al., Biochemistry (1991): vol. 30, p 7402; M. Schnolzer, et al., Int. J. Pept. Prot. Res. (1992): vol. 40, p 180-193; K. Rajarathnam, et al., Science (1994): vol. 264, p 90; R. E. Offord, “Chemical Approaches to Protein Engineering”, in Protein Design and the Development of New therapeutics and Vaccines, J. B. Hook, G. Poste, Eds., (Plenum Press, New York, 1990) pp. 253-282; C. J. A. Wallace, et al., J. Biol. Chem. (1992): vol. 267, p 3852; L. Abrahmsen, et al., Biochemistry (1991): vol. 30, p 4151; T. K. Chang, et al., Proc. Natl. Acad. Sci. USA (1994) 91: 12544-12548; M. Schnlzer, et al., Science (1992): vol., 3256, p 221; and K. Akaji, et al., Chem. Pharm. Bull. (Tokyo) (1985) 33: 184).

4. Structural Characterization of Polypeptides

In various embodiments, the methods of the invention involve determining structure information of a polypeptide using mass spectroscopy in combination with NMR or x-ray crystallography. In other embodiments, the methods of the invention involve use of mass spectroscopy, NMR and x-ray crystallography to structurally characterize a polypeptide.

In some instances, it may be advantageous to determine the structure of a polypeptide while complexed with another molecule, such as another polypeptide, nucleic acid or small molecule. In exemplary embodiments, the polypeptide is subjected to analysis by one or more of the structural techniques in both the presence and absence of another molecule so as to produce comparative data that is useful, for example, in designing modulators of the polypeptide or polypeptide complex.

In still other embodiments, the structure of two or more proteins are characterized and compared. Such data will be useful, for example, in determining the selectivity of a potential modulator for a particular polypeptide. For example, it may be desirable to identify an anti-bacterial therapeutic that modulates the activity of a bacterial polypeptide target but does not similarly affect the activity of a corresponding mammalian homolog or ortholog, e.g., the therapeutic is selective for the bacterial target. Comparison of structural information from two or more homologs or orthologs of interest will help to facilitate designing or identifying drugs with the desired selectivity. In certain embodiments, it may be desirable to determine the selectivity of a particular molecule by determine the ability of the molecule to bind and/or modulate the activity of at least 10, 25%, 30%, or 50% of the proteins in a defined proteome (e.g., membrane-associated and/or non-membrane associated proteins from a particular cell type, proteins from a particular organelle, proteins in a particular biosynthetic pathway, etc.).

(a) Analysis of Proteins by Mass Spectrometry

Mass spectrometry may be used to characterize the structure of a polypeptide in accordance with the methods of the invention. In particular, mass spectrometry can be used, for example, to determine the amino acid sequence, to obtain a peptide map, to identify post-translational modifications (e.g., phosphorylation, etc.) of a polypeptide, or to identifying regions of the polypeptide that interact with other molecules, including other polypeptides, nucleic acids and small molecules.

In certain embodiments, a polypeptide used in accordance with the methods of the invention is subjected to limited proteolysis prior to analysis by mass spectrometry. Limited proteolysis of a polypeptide may be used to identify and/or isolate stable domains of a protein that are suitable for structural characterization using NMR analysis or x-ray crystallography. Limited proteolysis of a polypeptide may be performed by incubating a protein with at least one concentration of a proteolytic enzyme for an amount of time suitable to produce proteolytic cleavage of the protein of interest. In certain embodiments, digestion of the polypeptide may be carried out by incubation with two or more proteolytic enzymes, at two or more concentrations of enzyme, and/or for varying amounts of time. Such reactions may be carried out in solution or by exposing the polypeptide to an immobilized proteolytic enzyme to facilitate isolation of the polypeptide fragments from the digestion mixture. The digestion products may be analyzed and/or isolated using electrophoretic or chromatographic techniques. Proteolytically stable fragments resulting from the enzymatic digestion may be identified based on the mass of the peptide as determined by mass spectrometry.

The stable proteolytic fragment may then be produced in suitable quantities to allow further structural characterization, for example, by NMR or x-ray crystallography. In certain embodiments, the proteolytic fragment is produced by expressing the full length protein, subjecting it limited proteolysis and then purifying the appropriate proteolytic fragment using electrophoresis, chromatography, or a combination thereof. Alternatively, identification of the boundaries of the proteolytic fragment within the sequence of the protein will allow recombinant production of the fragment. In this embodiment, a nucleic acid sequence encoding for the stable domain may be cloned into an expression vector, expressed under appropriate conditions and isolated using standard techniques.

In other embodiments, mass spectroscopy is used to obtain a peptide map and/or sequence information of a polypeptide. This information may be used to determine stable domains of the polypeptide by analysis of the amino acid sequence using, for example, various publicly available databases (e.g., http://smart.embl-heidelberg.de/). For example, based on the primary amino acid sequence, protein domains having a particular function or three dimensional structure may be identified. Polypeptide chains may fold into two or more domains joined by a flexible polypeptide chain segment. Such flexible regions may make it difficult to produce a crystallized polypeptide suitable for x-ray diffraction. Sequence analysis of the polypeptide will allow functional or structural domains to be identified and produced recombinantly in order to obtain a stable fragment of a polypeptide suitable for structural characterization using, for example, NMR or x-ray crystallography.

In other embodiments, mass spectroscopy may be used to identify post-translational modifications of a polypeptide. This may be achieved by obtaining the peptide map of a polypeptide before and after treatment of the polypeptide to remove or modify a particular type of post-translational modification. For example, if it is desirable to determine if a protein is phosphorylated, and at what sites in the polypeptide these phosphorylations occur, a peptide map of the polypeptide before and after treatment with a phosphatase may be generated. Each phosphorylation contained in a peptide fragment will shift the mass of the peptide by 80 Da. Identification of the particular residue(s) in the peptide which is modified by phosrphorylation may be determined by generating a peptide ladder to determine the amino acid sequence of the peptide. Similar analysis may be performed to analyze other post-translational modifications, such as, for example, glycosylation.

In still other embodiments, mass spectroscopy is used to identify regions of a polypeptide which interact with other molecules, including polypeptides, nucleic acids or small molecules. In certain embodiments, regions of a protein which interact with other molecules are determined by generating a peptide map of the protein in the presence and absence of the other molecule. Changes in the pattern of cleavage of the protein will allow identification of regions of the polypeptide that have become inaccessible to the proteolytic enzyme due to interaction with the other molecule. In other embodiments, regions of a protein which interact with other molecules may be identified by subjecting the protein to proteolytic digestion, preferably limited proteolytic digestion as described above, and using affinity chromatography to isolate fragments of the protein which interact with another molecule. For example, a protein digest may be run over a column functionalized with a test compound to isolate the fragments of the protein capable of interacting with the test compound. The protein fragments which bound to the column may then be eluted and subjected to analysis by mass spectrometry to identify the fragment of the protein which interacted with the test compound.

Typically, mass spectroscopy first requires protein isolation followed by either chemical or enzymatic digestion of the protein into smaller peptide fragments. For peptide mapping applications, the proteolytic digest should be essentially complete, e.g., resulting in at least about 70%, preferably at least 80%, 90%, 95% or 99% of the recombinant protein being digested. The proteolytic digests are also referred to as “peptide mixtures.”

A variety of proteolytic enzymes may be used to produce limited or complete digestion of polypeptides in accordance with the methods of the invention. Proteolytic enzymes which cut polypeptides into fragments appropriate for analysis by MS include, for example, aminopeptidase M; bromelain; carboxypeptidase A, B and Y; chymopapain; chymotrypsin; clostripain; collagenase; elastase; endoproteinase Arg-C, Glu-C, Asp-N and LysC; Factor Xa; ficin; Gelatinase; kallikrein; metalloendopeptinidase; papain; pepsin; plasmin; plasminogen; peptidase; pronase; proteinase A; proteinase K; subsilisin; thermolysin; thrombin; trypsin, or other suitable proteolytic enzymes.

If a tag has been used to facilitate protein expression or purification, a proteolytic enzyme which separates the tag from the recombinant polypeptide may be utilized. In certain embodiments, the proteolytic digestion can comprise one protease that removes the tag peptide and another protease that cuts the recombinant polypeptide into fragments of a size appropriate for MS. Alternatively, the same proteolytic enzyme may be used to remove the tag peptide and to cleave the recombinant protein into fragments.

In certain embodiments, the proteolytic enzyme may be attached to a solid support prior to incubation with the polypeptide to be digested. This allows easy removal of the proteolytic enzyme from the protein fragments prior to MS analysis, and thereby reduces background signals originating from the proteolytic enzyme. Solid supports are well known to those of skill in the art, and include any matrix used as a solid support for linking proteins. Supports, which can have a flat surface or a surface with structures, include, but are not limited to, beads such as silica gel beads, controlled pore glass beads, magnetic beads, Dynabeads, Wang resin; Merrifield resin, SEPHADEX/SEPHAROSE beads or cellulose beads; capillaries: flat supports such as glass fiber filters, glass surfaces, metal surfaces (including steel, gold silver, aluminum, silicon and copper), plastic materials (including multiwell plates or membranes (formed, for example, of polyethylene, polypropylene, polyamide, polyvinylidene difluoride), wafers, combs, pins or needles (including arrays of pins suitable for combinatorial synthesis or analysis) or beads in an array of pits; wells, particularly nanoliter wells, in flat surfaces, including wafers such as silicon wafers; and wafers with pits, with or without filter bottoms. A solid support is appropriately functionalized for conjugation of the proteolytic enzyme and can be of any suitable shape appropriate for the support.

A proteolytic enzyme can be conjugated directly to a solid support or can be conjugated indirectly through a functional group present either on the support, or a linker attached to the support, or the proteolytic enzyme or both. For example, a proteolytic enzyme can be immobilized to a solid support due to a hydrophobic, hydrophilic or ionic interaction between the support and the proteolytic enzyme.

A proteolytic enzyme also can be modified to facilitate conjugation to a solid support, for example, by incorporating a chemical or physical moiety at an appropriate position in the polypeptide, generally the C-terminus or N-terminus. It can also be modified at an amino acid in the peptide, for example, to a reactive side chain, or to the peptide backbone. It should be recognized, however, that a naturally occurring amino acid normally present in the proteolytic enzyme also can contain a functional group suitable for conjugating the polypeptide to the solid support. For example, a cysteine residue present in the polypeptide can be used to conjugate the polypeptide to a support containing a sulfhydryl group, for example, a support having cysteine residues attached thereto, through a disulfide linkage.

Digested proteins can be desalted and concentrated for increased MS, e.g., MALDI-TOF MS, sensitivity and resolution. The peptide fragments may be purified, for example by use of gel electrophoresis or column chromatography. A solid support that differentially binds the peptides and not reagents that were present in the proteolytic digestion may be used. The peptides can be eluted from the solid support into a small volume of a solution that is compatible with mass spectrometry (e.g., 50% acetonitrile/0.1% trifluoroacetic acid). Washing and purification procedures which remove reaction mixture components away from the peptides will increase the resolution of the spectrum resulting from mass spectrometric analysis of the recombinant polypeptide.

In a certain embodiment, MS samples can also be prepared by subjecting the proteolytically digested proteins to purification using Zip Tip_{C18} tips (Millipore), which are pipette tips that contain immobilized C18 attached at their very tip occupying about 0.5μl volume. For example, the Tips can be wet by aspirating and dispensing 100% methanol 5×; 2% acetonitrile/1% acetic acid (5×); 65% acetonitrile/1% acetic (5×); and 2% acetonitrile/1% acetic acid (5×). The Tips can then be placed back into the ZipTip rack; the digested proteins are then be bound to the ZipTips; the salts can be removed by washing the ZipTips with 2% acetonitrile/1% acetic acid (5×) and the digested proteins can be eluted by aspirating 65% acetonitrile/1% acetic acid. Multiple samples can be purified simultaneously using, e.g., an electronic pipettor, e.g., the 12-channel Biohit electronic pipettor (Biohit Inc., Neptune, N.J.).

The proteolytically digested proteins (or peptide mixtures) can also be conditioned prior to MS by treating the peptide mixtures with a cation exchange material or an anion exchange material, which can reduce the charge heterogeneity of the peptides, thereby reducing or eliminating peak broadening. In addition, contacting a polypeptide with an alkylating agent such as alkyliodide, iodoacetamide, iodoethanol, or 2,3-epoxy-1-propanol, for example, can prevent the formation of disulfide bonds in the polypeptide, thereby increasing resolution of a mass spectrum of the polypeptide. In certain embodiments, disulfide bonds of proteins are reduced, and the free thiols are alkylated after reduction, and preferably prior to digestion of the protein with protease. Reduction can be accomplished by incubation of the protein with a reducing agent, e.g., dithiothreitol. Likewise, charged amino acid side chains can be converted to uncharged derivatives by contacting the polypeptides with trialkylsilyl chlorides, thus reducing charge heterogeneity and increasing resolution of the mass spectrum.

Conditioning also can involve incorporating modified amino acids into the polypeptide, for example, mass modified amino acids, which can increase resolution of a mass spectrum. For example, the incorporation of a mass modified leucine residue in a polypeptide of interest can be useful for increasing the resolution (e.g., by increasing the mass difference) of a leucine residue from an isoleucine residue, thereby facilitating determination of an amino acid sequence of the polypeptide. A modified amino acid also can be an amino acid containing a particular blocking group, such as those groups used in chemical methods of amino acid synthesis. For example, the incorporation of a glutamic acid residue having a blocking group attached to the side chain carboxyl group can mass modify the glutamic acid residue and, provides the additional advantage of removing a charged group from the polypeptide, thereby further increasing resolution of a mass spectrum of a polypeptide containing the blocked amino acid. Incorporation of modified amino acids can be done at the time the protein is synthesized. The expression system that lends itself best to including such modified amino acids is an in vitro translation system, as described above.

The peptide mixtures are prepared for MS by mixing the peptide mixtures with a matrix appropriate for the particular MS used. The selection of a solution or reagent system, for example, an organic or inorganic solvent, will depend on the type of mass spectrometry performed, and is well known in the art (see, for example, Vorm et al., Anal. Chem. 66:3281 (1994), for MALDI; Valaskovic et al., Anal. Chem. 67:3802 (1995), for ESI). Mass spectrometry of peptides also is described, for example, in International PCT application No. WO 93/24834 to Chait et al. and U.S. Pat. No. 5,792,664.

A solvent is also selected so as to considerably reduce or fully exclude the risk that the peptides will be decomposed by the energy introduced for the vaporization process. A reduced risk of peptide decomposition can be achieved, for example, by embedding the sample in a matrix, which can be an organic compound such as a sugar, for example, a pentose or hexose, or a polysaccharide such as cellulose. Such compounds are decomposed thermolytically into CO₂ and H₂O such that no residues are formed that can lead to chemical reactions. The matrix also can be an inorganic compound such as nitrate of ammonium, which is decomposed essentially without leaving any residue. Use of these and other solvents is known to those of skill in the art (see, e.g., U.S. Pat. No. 5,062,935).

The peptide mixture and matrix are then applied to a plate for MS analysis, e.g., a metal target plate, according to methods known in the art. In a preferred embodiment, the plates are anchor plates, e.g., plates having a hydrophobic coating and hydrophilic patches (“anchors”). The hydrophobic coating can be, e.g., Teflon. An exemplary plate that can be used is the Bruker Daltonics's Anchor Chip™. Samples can be applied to the plates according to the manufacturer's instructions. Briefly, μl sample droplets are deposited onto the plates. The droplets shrink during solvent evaporation and center themselves onto the anchor positions. This allows the peptides to be concentrated in smaller spots and thereby increases the sensitivity of MS detection. Samples can be spotted automatically, e.g., by SpotBot™ Personal Microarrayer (TeleChem International, Inc.).

The peptide mixtures may also be subjected to a reverse phase column and elution of the peptides from the column directly into a mass spectrometer using an electrospray or nano-electrospray sample introduction interface. For example, peptides may be eluted directly into an ion trap or triple quadrupole mass spectrometer.

Mass spectrometer formats for use in analyzing the peptide mixtures include ionization (I) techniques, such as, but not limited to, matrix assisted laser desorption (MALDI), continuous or pulsed electrospray (ESI) and related methods such as ionspray or thermospray, and massive cluster impact (MCI). Such ion sources can be matched with detection formats, including linear or non-linear reflectron time-of-flight (TOF), single or multiple quadrupole, single or multiple magnetic sector, Fourier transform ion cyclotron resonance (FTICR), ion trap, and combinations thereof such as ion-trap/time-of-flight. For ionization, numerous matrix/wavelength combinations (MALDI) or solvent combinations (ESI) can be employed. Sub-attomole levels of protein have been detected, for example, using ESI mass spectrometry (Valaskovic, et al., Science 273:1199-1202 (1996)) and LDI mass spectrometry (Li et al., J. Am. Chem. Soc. 118:1662-1663(1996)).

Accordingly, the following mass spectrometers may be used in accordance with the methods of the present invention: triple quadrupole mass spectrometers, magnetic sector instruments (magnetic tandem mass spectrometer, JEOL, Peabody, Mass), ionspray mass spectrometers (Bruins et al., Anal Chem. 59:2642-2647, 1987; Fenn et al. J. Phys. Chem. 88:4451-59 (1984); PCT Application No. WO 90/14148; Smith et al., Anal. Chem. 62:882-89 (1990); Ardrey, Electrospray Mass Spectrometry, Spectroscopy Europe 4:10-18 (1992)); electrospray mass spectrometers (Fenn et al., Science 246:64-71, 1989); laser desorption time-of-flight mass spectrometers (Karas and Hillenkamp, Anal. Chem. 60:2299-2301 (1988), and Fourier Transform Ion Cyclotron Resonance Mass Spectrometer (Extrel Corp., Pittsburgh, Mass.). Generally, the methods of the invention can be practiced with any mass spectrometer that has the capability of measuring peptide masses with high mass accuracy, precision, and resolution, as well as the capability of measuring the masses of fragments generated from a specific peptide when analyzed under conditions that induce dissociation of the peptide.

In an exemplary embodiment, matrix assisted laser desorption (MALDI) is used in conjunction with methods described herein. Peptide masses are typically accurately measured using a MALDI-TOF or a MALDI-Q-Star mass spectrometer down to the low ppm (parts per million) precision level. MALDI ionization is a technique in which samples of interest, in this case peptides, are co-crystallized with an acidified matrix. The matrix is a small molecule, which absorbs at a specific wavelength, generally in the ultraviolet (UV) range and dissipates the absorbed energy thermally. Typically, a pulse laser beam is used to transfer energy rapidly (e.g., a few ns) to the matrix. This rapid transfer of energy causes the matrix to rapidly dissociate from the surface generating a plume of matrix and the co-crystallized analytes into the gas phase. It is not clear if the analytes acquire their charge during the desorption process or after entering the gas plume of molecules by interacting with the matrix molecules. However, the end result is a small pocket of charged analytes that are present in the gas phase. To date, MALDI has been predominantly coupled in-line with time of flight (TOF) mass spectrometers.

The function of a time of flight mass spectrometer is to measure the time that analytes take to travel across a fixed path length (the TOF tube or chamber). The charged analytes present in the plume are therefore transferred to the TOF tube after an appropriate time delay. In order to move the analytes into the TOF tube, a high voltage is applied to the MALDI plate generating a strong electric field between the plates and the entrance of the TOF chamber. Smaller analytes will reach the entrance of the chamber more rapidly than larger analytes (i.e. constant kinetic energy applied, generating different velocity for the analytes). Once in flight, the analytes are in a field-free region and separate along the tube while moving toward the detector. Again, analytes of lesser mass move along the tube faster and reach the detector prior to analytes of greater mass. The detector is in tune with the laser shots and time delay, and measures the peptide and protein ions as they arrive over time. When the mass range is calibrated by using standards of known mass and charge, the time of flight for a given ion can be converted to masses. The end result is a spectrum comparing observed intensity versus ion (protein or polypeptide) mass. MALDI-TOF mass spectrometry has been described by Hillenkamp et al. (“Matrix Assisted UV-Laser Desorption/Ionization: A New Approach to Mass Spectrometry of Large Biomolecules, Biological Mass Spectrometry” (Burlingame and McCloskey, eds., Elsevier Science Publ. (1990), pp. 49-60).

MALDI-TOF MS is easily performed with modem mass spectrometers. Typically the samples of interest, in this case peptides, are mixed with a matrix mixture and successively spotted onto a polished stainless steel plate (MALDI plate). Commercially available MALDI plates can hold multiple samples per plate and are compatible with high throughput formats, e.g., 96 and 384 sample arrangements. The MALDI plate is then installed into the vacuum chamber of a MALDI mass spectrometer. The pulsed laser is activated and the time of flight acquisition triggered. An MS spectrum containing the mass to charge ratios of the peptides is then generated. The charge of molecules ionized by MALDI is typically 1.

Methods for performing MALDI are well known to those of skill in the art. Numerous methods for improving resolution are also known. For example, resolution in MALDI TOF mass spectrometry can be improved by reducing the number of high energy collisions during ion extraction (see, e.g., Juhasz et al. (1996) Analysis. Anal. Chem. 68:941-946, see also, e.g., U.S. Pat. No. 5,777,325, 5,742,049, 5,654,545, 5,641,959, 5,654,545, 5,760,393 and 5,760,393 for descriptions of MALDI and delayed extraction protocols).

MALDI-TOF is useful for high throughput procedures, since it takes approximately 30 seconds to analyze a sample by MALDI-TOF in an automated procedure, whereas it takes approximately one hour to merely introduce samples into the other kinds of instruments via micro-capillary HPLC. In addition, MALDI-TOF yields a high accuracy peptide mass spectrum (Patterson, Electrophoresis 1995, 16, 1104-14). This sensitive method is able to characterize proteins that are present at very low concentration, as low as sub-picomole levels.

Tandem mass spectrometry or post source decay can be used for proteins that cannot be identified by peptide-mass matching or to confirm the identity of proteins that are tentatively identified by an error-tolerant peptide mass search, described above. This method combines two consecutive stages of mass analysis to detect secondary fragment ions that are formed from a particular precursor ion. The first stage serves to isolate a particular ion of a particular peptide (polypeptide) of interest based on its m/z. The second stage is used to analyze the product ions formed by spontaneous or induced fragmentation of the selected ion precursor. Interpretation of the resulting spectrum provides limited sequence information for the peptide of interest. However, it is faster to use the masses of the observed peptide fragment ions to search an appropriate protein sequence database and identify the protein as described in Griffin et al, Rapid Commun. Mass. Spectrom. 1995, 9,1546-51.

The identity of a polypeptide analyzed by mass spectroscopy may be determined by using position and height of the peptide peaks to search protein/DNA databases in a method often called peptide mass fingerprinting. In this approach protein entries in the databases are ranked according to the number of peptide masses that match to their predicted trypsin digestion pattern. The peptide masses can be searched against in-house proprietary and public databases using a correlative mass matching algorithm. Statistical analysis can be performed upon each protein match to determine the validity of the match. Typical constraints include error tolerances within 0.1 Da for monoisotopic peptide masses. Cysteines are alkylated and searched as carboxyamidomethyl modifications. Identified proteins can be stored automatically in a relational database, e.g., having software links to SDS-PAGE images or ligand sequences. Often, even a partial peptide map of a protein is specific enough for identification of the protein. If no match is found, a more error-tolerant search can be used, for example using fewer peptides or allowing a larger margin for error. In these cases the tentative identity of the interacting protein should be confirmed by a second method.

Commercially available and in-house developed software packages can be utilized to calculate and/or summarize these characteristics/properties in database format. Protein identification and quantification can be obtained within minutes from MALDI-TOF MS generated data that is analyzed by both commercially available and in-house developed software packages.

In an exemplary embodiment, the KNEXUS/MS software (Proteometrics LLC, New York, N.Y.) is used. This software interprets and translates the raw mass spectra files and stores the results. Knexus uses the ProFound™ search engine (Proteometrics LLC, New York, N.Y.) for searching protein sequences from database matches, the CLIENT M/Z (Proteometrics LLC, New York, N.Y.) application to extract peak masses from spectra and the Sonar ms/ms™ (Proteometrics) engine for analyzing information from tandem mass spectrometry. The ProFound™ search engine identifies proteins based on statistics that clearly indicate the probability that a protein identification result is caused by random statistical coincidence. ProFound™ mimics the experiment by calculating the proteolytic peptide masses for all protein sequences in the database and creating a theoretical mass spectrum for each protein sequence. Each theoretical mass spectrum is compared to the experimental mass spectrum, and a score that reflects the similarity is calculated using Bayesian statistics. The algorithm uses detailed information about each individual protein sequence and incorporates additional experimental information (e.g. peptide fragment mass information, amino acid composition or sequence information) when available. Published algorithms provide accurate matches of fragments to proteins, ranking the matches using Bayesian statistics, and a display of errors (so that a requirement for the recallibration of the mass spectrometry spectra may be rapidly diagnosed). Hyperlinks in the Knexus Report connect to database files for the proteins, and connect directly to the Protein Analysis Work Sheet (PAWS).

Software for identifying proteins and peptide fragments from tandem mass spectrometry, Quadrapole, QTOF, TOF/TOF, Ion Trap and ESI-Nanospray are also publicly or commercially available, e.g., from Proteometrics (New York, N.Y.). For example, tandem mass spectra data can be analyzed with the Sonar ms/ms™ algorithm. Another algorithm useful for protein analysis is m/z (em-over-zee), a freeware program provided by Proteometrics (New York, N.Y.) for the analysis of protein mass spectra.

Another useful resource for protein analysis is Biopolymer markup language (BIOML) from Proteometrics (New York, N.Y.), which is a browser that allows the full specification of all experimental information known about molecular entities composed of biopolymers, for example, proteins and genes. BIOML provides an extensible framework for the annotation of biopolymers and to provide a common vehicle for exchanging this information between scientists using the World Wide Web.

(b) Analysis of Proteins by Nuclear Magnetic Resonance (NMR)

NMR may be used to characterize the structure of a polypeptide in accordance with the methods of the invention. In particular, NMR can be used, for example, to determine the three dimensional structure, the conformational state, the aggregation level, the state of protein folding/unfolding or the dynamic properties of a polypeptide. Changes in these properties due to interaction with other molecules can also be monitored using NMR. Thus, the invention also encompasses methods for detecting, designing and characterizing interactions between a polypeptide and another molecule, including polypeptides, nucleic acids and small molecules utilizing NMR techniques.

Polypeptides in aqueous solution usually populate an ensemble of 3-dimensional (3D) structures which can be determined by NMR. The 2-dimensional ¹H-¹⁵N HSQC (Heteronuclear Single Quantum Correlation) spectrum provides a diagnostic fingerprint of conformational state, aggregation level, state of protein folding, and dynamic properties of a polypeptide (Yee et al, PNAS 99, 1825-30 (2002)). When the polypeptide is a stable globular protein or domain of a protein, then the ensemble of solution structures is one of very closely related conformations. In this case one peak is expected for each non-proline residue with a dispersion of resonance frequencies with roughly equal intensity. Additional pairs of peaks from side-chain NH2 groups are also often observed, and correspond to approximately the number of Gln and Asn residues in the protein. This type of HSQC spectra usually indicates that the protein is amenable to structure determination by NMR methods.

If the HSQC spectrum shows well-dispersed peaks but there are either too few or too many in number, and/or the peak intensities differ throughout the spectrum, then the protein likely does not exist in a single globular conformation and is less amenable to NMR structure determination. Such spectral features are indicative of conformational heterogeneity with slow or nonexistent inter-conversion between states (too many peaks) or the presence of dynamic processes on an intermediate timescale that can broaden and obscure the NMR signals. Proteins with this type of spectrum can sometimes be stabilized into a single conformation more amenable to NMR structure determination by changing either the protein construct, the solution conditions, temperature or by binding of another molecule.

The ¹H-¹⁵N HSQC can also indicate whether a protein is has formed large nonspecific aggregates or has dynamic properties that make it unsuitable for structure determination or characterization by NMR. Polypeptides with these properties generally display ¹H-¹⁵N HSQC spectra with very broad peaks often with little spectral dispersion in which very few individual peaks can be identified.

Finally, proteins that are largely “unfolded” having very little regular secondary structure result in ¹H-¹⁵N HSQC spectra in which the peaks are all very narrow and intense, but have very little spectral dispersion in the ¹⁵N-dimension. This reflects the fact that many or most of the amide groups of amino acids in unfolded polypeptides are solvent exposed and experience similar chemical environments resulting in similar ¹H chemical shifts.

The use of the ¹H-¹⁵N HSQC, can thus allow the rapid characterization of the conformational state, aggregation level, state of protein folding, and dynamic properties of a polypeptide. This affords a rapid method for screening the characteristics of many polypeptides (Yee et al, PNAS 99, 1825-30 (2002)). Additionally other 2D spectra such as ¹H-¹³C HSQC, or HNCO spectra can also be used in a similar manner. Further use of the ¹H-¹⁵N HSQC combined with relaxation measurements can reveal the molecular rotational correlation time and dynamic properties of polypeptides. The rotational correlation time is proportional to size of the protein and therefore can reveal if it forms specific homo-oligomers such as homodimers, homotetramers, etc.

NMR analysis of a polypeptide in the presence and absence of a test compound (e.g., a polypeptide, nucleic acid or small molecule) may be used to characterize interactions between a polypeptide and another molecule. Because the ¹H-¹⁵N HSQC spectrum and other simple 2D NMR experiments can be obtained very quickly (on the order of minutes depending on protein concentration and NMR instrumentation), they are very useful for rapidly testing whether a polypeptide is able to bind to another molecule such as another protein, nucleic acid or small molecule. Changes in the resonance frequency (in one or both dimensions) of one or more peaks in the HSQC spectrum indicate an interaction with another molecule (Ref. Fesik et al's patent on SAR by NMR). Often only a subset of the peaks will have changes in resonance frequency upon binding to anther molecule, allowing one to map onto the 3D structure those residues directly involved in the interaction or involved in conformational changes as a result of the interaction. If the interacting molecule is relatively large (protein or nucleic acid) the peak widths will also broaden due to the increased rotational correlation time of the complex. In some cases the peaks involved in the interaction may actually disappear from the NMR spectrum if the interacting molecule is “in intermediate exchange on the NMR timescale (ie, exchanging on and off the polypeptide at a frequency that is similar to the resonance frequency of the monitored nuclei).

Briefly, the NMR technique involves placing the material to be examined (usually in a suitable solvent) in a powerful magnetic field and irradiating it with radio frequency (rf) electromagnetic radiation. The nuclei of the various atoms will align themselves with the magnetic field until energized by the rf radiation. They then absorb this resonant energy and re-radiate it at a frequency dependent on i) the type of nucleus and ii) its atomic environment. Moreover, resonant energy may be passed from one nucleus to another, either through bonds or through three-dimensional space, thus giving information about the environment of a particular nucleus and nuclei in its vicinity.

However, it is important to recognize that not all nuclei are NMR active. Indeed, not all isotopes of the same element are active. For example, whereas “ordinary” hydrogen, ¹H, is NMR active, heavy hydrogen (deuterium), ²H, is not active in the same way. Thus, any material that normally contains ¹H hydrogen may be rendered “invisible” in the hydrogen NMR spectrum by replacing all or almost all the ¹H hydrogens with ²H. It is for this reason that NMR spectroscopic analyses of water-soluble materials frequently are performed in ²H₂O to eliminate the water signal.

Conversely, “ordinary” carbon, ¹²C, is NMR inactive whereas the stable isotope, ¹³C, present to about 1% of total carbon in nature, is active. Similarly, while “ordinary” nitrogen, ¹⁴N, is NMR active, it has undesirable properties for NMR and resonates at a different frequency from the stable isotope ¹⁵N, present to about 0.4% of total nitrogen in nature.

By labeling proteins with ¹⁵N and ¹⁵N/¹³C, it is possible to conduct analytical NMR of macromolecules with weights of 15 kD and 40 kD, respectively. More recently, partial deuteration of the protein in addition to ¹³C- and ¹⁵N-labeling has increased the possible weight of proteins and protein complexes for NMR analysis still further, to approximately 60-70 kD. See Shan et al., J. Am. Chem.Soc., 118:6570-6579 (1996); L. E. Kay, Methods Enzymol., 339:174-203 (2001); and K. H. Gardner & L. E. Kay, Annu Rev Biophys Biomol Struct., 27:357-406 (1998); and references cited therein.

Isotopic substitution is usually accomplished by growing a bacterium or yeast or other type of cultured cells, transformed by genetic engineering to produce the protein of choice, in a growth medium containing ¹³C-, ¹⁵N- and/or ²H-labeled substrates. In practice, bacterial growth media usually consist of ¹³C-labeled glucose and/or 15N-labeled ammonium salts dissolved in D₂O where necessary. Kay, L. et al., Science, 249:411 (1990) and references therein and Bax, A., J. Am. Chem. Soc., 115, 4369 (1993). More recently, isotopically labeled media especially adapted for the labeling of bacterially produced macromolecules have been described. See U.S. Pat. No. 5,324,658.

The goal of these methods has been to achieve universal and/or random isotopic enrichment of all of the amino acids of the protein. By contrast, methods allow only certain residues to be relatively enriched in ¹H, ²H, ¹³C and ¹⁵N. For example, Kay et al., J. Mol. Biol., 263, 627-636 (1996) and Kay et al., J. Am. Chem. Soc., 119, 7599-7600 (1997) have described methods whereby isoleucine, alanine, valine and leucine residues in a protein may be labeled with ²H, ¹³C and ¹⁵N, but specifically labeled with ¹H at the terminal methyl position. In this way, study of the proton-proton interactions between some of the hydrophobic amino acids may be facilitated. Similarly, a cell-free system has been described by Yokoyama et al., J. Biomol. NMR, 6(2), 129-134 (1995)., wherein a transcription-translation system derived from E. coli was used to express human Ha-Ras protein incorporating ¹⁵N serine and/or aspartic acid.

Techniques for producing isotopically labeled proteins and macromolecules, such as glycoproteins, in mammalian or insect cells have been described. See U.S. Pat. Nos. 5,393,669 and 5,627,044; Weller, C. T., Biochem., 35, 8815-23 (1996) and Lustbader, J. W., J.Biomol. NMR, 7, 295-304 (1996).

The 3D structure of stable globular proteins can be determined through a series of well-described procedures. For a general review of structure determination of globular proteins in solution by nuclear magnetic resonance spectroscopy, see Wüthrich, Science 243: 45-50 (1989). See also, Billeter et al., J. Mol. Biol. 155: 321-346 (1982). Current methods for structure determination usually require the complete or nearly complete sequence-specific assignment of ¹H-resonance frequencies of the protein and subsequent identification of approximate inter-hydrogen distances (from nuclear Overhause effect (NOE) spectra) for use in restrained molecular dynamics calculations of the protein conformation. One approach for the analysis of NMR resonance assignments was first outlined by Wüthrich, Wagner and co-workers (Wüthrich, “NMR or proteins and nucleic acids” Wiley, New York, N.Y. (1986); Wüthrich, Science 243: 45-50 (1989); Billeter et al., J. Mol. Biol. 155: 321-346 (1982)). Newer methods for determining the structures of globular proteins include the use of residual dipolar coupling restraints (Tian, Valafar & Prestegard, A dipolar coupling based strategy for simultaneous resonance assignment and structure determination of protein backbones, J Am Chem Soc. 2001 Nov 28;123(47):11791-6.; Bax et al, Dipolar couplings in macromolecular structure determination Methods Enzymol. 2001;339:127-74.) and empirically derived conformational restraints (Zweckstetter & Bax, Single-step determination of protein substructures using dipolar couplings: aid to structural genomics, J Am Chem Soc. 2001 Sep 26;123(38):9490-1). Most recently Grishaev and Llinas (PNAS in press) have shown that it may be possible to determine 3D structures of globular proteins using only un-assigned NOE measurements, avoiding the labor intensive resonance assignment steps, which would make the procedure much faster.

NMR may also be used to determine ensembles of many inter-converting “unfolded” conformations (Choy and Forman-Kay, Calculation of ensembles of structures representing the unfolded state of an SH3 domain. J Mol Biol. 2001 May 18;308(5):1011-32).

In another embodiment, the invention provides a screening method for identifying small molecular weight compounds, or ligands, capable of interacting with a polypeptide of the invention. In one example, the screening process begins with the generation or acquisition of either a T₂-filtered or a diffusion-filtered one-dimensional proton spectrum of the compound or mixture of compounds. Means for generating T₂-filtered or diffusion-filtered one-dimensional proton spectra are well known in the art (see, e.g., S. Meiboom and D. Gill, Rev. Sci. Instrum. 29:688(1958), S. J. Gibbs and C. S. Johnson, Jr. J. Main. Reson. 93:395-402 (1991) and A. S. Altieri, et al. J. Am. Chem. Soc. 117: 7566-7567 (1995)).

To facilitate the acquisition of NMR data on a large number of compounds (e.g., a database of synthetic or naturally occurring small organic compounds), a sample changer may be employed. Using the sample changer, a larger number of samples, numbering 60 or more, may be run unattended. To facilitate processing of the NMR data, computer programs are used to transfer and automatically process the multiple one-dimensional NMR data.

Following acquisition of the first spectrum for the test compounds, the ¹⁵N- or ¹³C-labeled polypeptide is exposed to one or more test compounds. Where more than one test compound is to be tested simultaneously, it is preferred to use a database of compounds such as a plurality of small molecules. Such molecules are typically dissolved in perdeuterated dimethylsulfoxide. The compounds in the database may be purchased from vendors or created according to desired needs.

Individual compounds may be selected inter alia on the basis of size (molecular weight=100−300) and molecular diversity. Compounds in the collection may have different shapes (e.g., flat aromatic rings(s), puckered aliphatic rings(s), straight and branched chain aliphatics with single, double, or triple bonds) and diverse functional groups (e.g., carboxylic acids, esters, ethers, amines, aldehydes, ketones, and various heterocyclic rings) for maximizing the possibility of discovering compounds that interact with widely diverse binding sites of a subject polypeptide.

The NMR screening process of the present invention utilizes a range of test compound concentrations, e.g., from about 0.05 to about 1.0 mM. At those exemplary concentrations, compounds which are acidic or basic may significantly change the pH of buffered protein solutions. Chemical shifts are sensitive to pH changes as well as direct binding interactions, and “false positive” chemical shift changes, which are not the result of test compound binding but of changes in pH, may therefore be observed. It may therefore be necessary to ensure that the pH of the buffered solution does not change upon addition of the test compound.

Following exposure of the test compounds to a polypeptide (e.g., the target molecule for the experiment) a second one-dimensional T₂- or diffusion-filtered spectrum is generated. For the T₂-filtered approach, that second spectrum is generated in the same manner as set forth above. The first and second spectra are then compared to determine whether there are any differences between the two spectra. Differences in the one-dimensional T₂-filtered spectra indicate that the compound is binding to, or otherwise interacting with, the target molecule. Those differences are determined using standard procedures well known in the art. For the diffusion-filtered method, the second spectrum is generated by looking at the spectral differences between low and high gradient strengths—thus selecting for those compounds whose diffusion rates are comparable to that observed in the absence of target molecule.

To discover additional molecules that bind to the protein, molecules are selected for testing based on the structure/activity relationships from the initial screen and/or structural information on the initial leads when bound to the protein. By way of example, the initial screening may result in the identification of compounds, all of which contain an aromatic ring. The second round of screening would then use other aromatic molecules as the test compounds.

In another embodiment, the methods of the invention utilize a process for detecting the binding of one ligand to a polypeptide in the presence of a second ligand. In accordance with this embodiment, a polypeptide is bound to the second ligand before exposing the polypeptide to the test compounds.

See also: U.S. Pat. Nos. 5,668,734; 6,194,179; 6,162,627; 6,043,024; 5,817,474; 5,891,642; 5,989,827; 5,891,643; 6,077,682; WO 00/05414; WO 99/22019; Cavanagh, et al., Protein NMR Spectroscopy, Principles and Practice, 1996, Academic Press; Clore, et al., NMR of Proteins. In Topics in Molecular and Structural Biology, 1993, S. Neidle, Fuller, W., and Cohen, J. S., eds., Macmillan Press, Ltd., London; and Christendat et al., Nature Structural Biology 7: 903-909 (2000).

(c) Analysis of Proteins by X-ray Crystallography

X-ray crystallogray may be used to characterize the structure of a polypeptide in accordance with the methods of the invention. In particular, x-ray diffraction of a crystallized form of a polypeptide can be used, for example, to determine the three dimensional structure of a polypeptide or to determine the space group of the crystal of the polypeptide. The invention also encompasses methods for detecting, designing and characterizing interactions between a polypeptide and another molecule, including polypeptides, nucleic acids and small molecules utilizing x-ray crystallographic techniques.

Exemplary methods for obtaining the three dimensional structure of the crystalline form of a molecule or complex are described herein and, in view of this specification, variations on these methods will be apparent to those skilled in the art (see Ducruix and Geige 1992, IRL Press, Oxford, England).

X-ray crystallography techniques generally require that the protein molecules be available in the form of a crystal. Crystals may be grown from a solution containing a purified polypeptide, or a fragment thereof (e.g., a stable domain), by a variety of conventional processes. These processes include, for example, batch, liquid, bridge, dialysis, vapour diffusion (e.g., hanging drop or sitting drop methods). (See for example, McPherson, 1982 John Wiley, New York; McPherson, 1990, Eur. J. Biochem. 189: 1-23; Webber. 1991, Adv. Protein Chem. 41:1-36). In certain embodiments, native crystals of the invention may be grown by adding precipitants to the concentrated solution of the polypeptide. The precipitants are added at a concentration just below that necessary to precipitate the protein. Water may be removed by controlled evaporation to produce precipitating conditions, which are maintained until crystal growth ceases. The formation of crystals is dependent on a number of different parameters, including pH, temperature, protein concentration, the nature of the solvent and precipitant, as well as the presence of added ions or ligands to the protein. In addition, the sequence of the polypeptide being crystallized will have a significant affect on the success of obtaining crystals. Many routine crystallization experiments may be needed to screen all these parameters for the few combinations that might give crystal suitable for x-ray diffraction analysis (See, for example, Jancarik, J & Kim, S. H., J. Appl. Cryst. 1991 24: 409-411). Crystallization robots may automate and speed up the work of reproducibly setting up large number of crystallization experiments. Once some suitable set of conditions for growing the crystal are found, variations of the condition may be systematically screened in order to find the set of conditions which allows the growth of sufficiently large, single, well ordered crystals. In certain instances, a polypeptide is co-crystallized with a compound that stabilizes the polypeptide.

In certain embodiments of the methods of the subject invention, it may be useful to determine the three dimensional structure of a crystallized polypeptide in the presence of another molecule, such as another polypeptide, nucleic acid or small molecule. In such embodiments, a polypeptide may be co-crystallized with another molecule in order to provide a crystal suitable for determining the structure of the complex. Alternatively, a crystal of the polypeptide may be soaked in a solution containing the other molecule in order to form co-crystals by diffusion of the other molecule into the crystal of the polypeptide. In exemplary embodiments, the structure of the polypeptide obtained in the presence and absence of another molecule may be compared to determine structural information about the polypeptide and aid in identification of druggable regions.

A number of methods are available to produce suitable radiation for X-ray diffraction. For example, x-ray beams may be produced by synchrotron rings where electrons (or positrons) are accelerated through an electromagnetic field while traveling at close to the speed of light. Because the admitted wavelength may also be controlled, synchrotrons may be used as a tunable x-ray source (Hendrickson W A., Trends Biochem Sci 2000 Dec; 25(12):637-43). For less conventional Lane diffraction studies, polychromatic x-rays covering a broad wavelength window are used to observe many diffraction intensities simultaneously (Stoddard, B. L., Curr. Opin. Struct Biol 1998 Oct; 8(5):612-8). Neutrons may also be used for solving protein crystal structures (Gutberlet T, Heinemann U & Steiner M., Acta Crystallogr D 2001;57:349-54).

Before data collection commences, a protein crystal may be frozen to protect it from radiation damage. A number of different cryo-protectants may be used to assist in freezing the crystal, such as methyl pentanediol (MPD), isopropanol, ethylene glycol, glycerol, formate, citrate, mineral oil, or a low-molecular-weight polyethylene glycol (PEG). As an alternative to freezing the crystal, the crystal may also be used for diffraction experiments performed at temperatures above the freezing point of the solution. In these instances, the crystal may be protected from drying out by placing it in a narrow capillary of a suitable material (generally glass or quartz) with some of the crystal growth solution included in order to maintain vapour pressure.

X-ray diffraction results may be recorded by a number of ways know to one of skill in the art. Examples of area electronic detectors include charge coupled device detectors, multi-wire area detectors and phosphoimager detectors (Amemiya, Y, 1997. Methods in Enzymology, Vol. 276. Academic Press, San Diego, pp. 233-243; Westbrook, E. M., Naday, I. 1997. Methods in Enzymology, Vol. 276. Academic Press, San Diego, pp. 244-268; 1997. Kahn, R. & Fourme, R. Methods in Enzymology, Vol. 276. Academic Press, San Diego, pp. 268-286).

A suitable system for laboratory data collection might include a Bruker AXS Proteum R system, equipped with a copper rotating anode source, Confocal Max-Flux™ optics and a SMART 6000 charge coupled device detector. Collection of X-ray diffraction patterns are well documented by those skilled in the art (See, for example, Ducruix and Geige, 1992, IRL Press, Oxford, England).

The theory behind diffraction by crystal upon exposure to x-rays is well known. Because phase information is not directly measured in the diffraction experiment, and is needed to reconstruct the electron density map, methods that can recover this missing information are required. One method of solving structures ab initio are the real/reciprocal space cycling techniques. Suitable real/reciprocal space cycling search programs include shake-and-bake (Weeks C M, DeTitta G T, Hauptman H A, Thuman P, Miller R Acta Crystallogr A 1994; V50: 210-20).

Other methods for deriving phases may also be needed. These techniques generally rely on the idea that if two or more measurements of the same reflection are made where strong, measurable, differences are attributable to the characteristics of a small subset of the atoms alone, then the contributions of other atoms can be, to a first approximation, ignored, and positions of these atoms may be determined from the difference in scattering by one of the above techniques. Knowing the position and scattering characteristics of those atoms one may calculate what phase the overall scattering must have had to produce the observed differences.

One version of this technique is isomorphous replacement technique, which requires the introduction of new, well ordered, x-ray scatterers into the crystal. These additions are usually heavy metal atoms, (so that they make a significant difference in the diffraction pattern); and if the additions do not change the structure of the molecule or of the crystal cell, the resulting crystals should be isomorphous. Isomorphous replacement experiments are usually performed by diffusing different heavy-metal metals into the channels of a pre-existing protein crystal. Growing the crystal from protein that has been soaked in the heavy atom is also possible (Petsko, G. A., 1985. Methods in Enzymology, Vol. 114. Academic Press, Orlando, pp. 147-156). Alternatively, the heavy atom may also be reactive and attached covalently to exposed amino acid side chains (such as the sulfur atom of cysteine) or it may be associated through non-covalent interactions. It is sometimes possible to replace endogenous light metals in metallo-proteins with heavier ones, e.g., zinc by mercury, or calcium by samarium (Petsko, G. A., 1985. Methods in Enzymology, Vol. 114. Academic Press, Orlando, pp. 147-156). Exemplary sources for such heavy compounds include, without limitation, sodium bromide, sodium selenate, trimethyl lead actate, mercuric chloride, methyl mercury acetate, platinum tetracyanide, platinum tetrachloride, nickel chloride, and europium chloride.

A second technique for generating differences in scattering involves the phenomenon of anomalous scattering. X-rays that cause the displacement of an electron in an inner shell to a higher shell are subsequently rescattered, but there is a time lag that shows up as a phase delay. This phase delay is observed as a (generally quite small) difference in intensity between reflections known as Friedel mates that would be identical if no anomalous scattering were present. A second effect related to this phenomenon is that differences in the intensity of scattering of a given atom will vary in a wavelength dependent manner, given rise to what are known as dispersive differences. In principle anomalous scattering occurs with all atoms, but the effect is strongest in heavy atoms, and may be maximized by using x-rays at a wavelength where the energy is equal to the difference in energy between shells. The technique therefore requires the incorporation of some heavy atom much as is needed for isomorphous replacement, although for anomalous scattering a wider variety of atoms are suitable, including lighter metal atoms (copper, zinc, iron) in metallo-proteins. One method for preparing a protein for anomalous scattering involves replacing the methionine residues with selenium containing seleno-methionine. Soaks with halide salts such as bromides and other non-reactive ions may also be effective (Dauter Z, Li M, Wlodawer A., Acta Crystallogr D 2001; 57: 239-49).

In another process, known as multiple anomalous scattering or MAD, two to four suitable wavelengths of data are collected. (Hendrickson, W. A. and Ogata, C. M. 1997 Methods in Enzymology 276, 494-523). Phasing by various combinations of single and multiple isomorphous and anomalous scattering are possible too. For example, SIRAS (single isomorphous replacement with anomalous scattering) utilizes both the isomorphous and anomalous differences for one derivative to derive phases. More traditionally, several different heavy atoms are soaked into different crystals to get sufficient phase information from isomorphous differences while ignoring anomalous scattering, in the technique known as multiple isomorphous replacement (MIR) (Petsko, G. A., 1985. Methods in Enzymology, Vol. 114. Academic Press, Orlando, pp. 147-156).

Additional restraints on the phases may be derived from density modification techniques. These techniques use either generally known features of electron density distribution or known facts about that particular crystal to improve the phases. For example, because protein regions of the crystal scatter more strongly than solvent regions, solvent flattening/flipping may be used to adjust phases to make solvent density a uniform flat value (Zhang, K. Y. J., Cowtan, K. and Main, P. Methods in Enzymology 277, 1997 Academic Press, Orlando pp 53-64). If more than one molecule of the protein is present in the asymmetric unit, the fact that the different molecules should be virtually identical may be exploited to further reduce phase error using non-crystallographic symmetry averaging (Villieux, F. M. D. and Read, R. J. Methods in Enzymology 277, 1997 Academic Press, Orlando ppl8-52). Suitable programs for performing these processes include DM and other programs of the CCP4 suite (Collaborative Computational Project, Number 4. 1994. Acta Cryst. D50, 760-763) and CNX.

The unit cell dimensions, symmetry, vector amplitude and derived phase information can be used in a Fourier transform function to calculate the electron density in the unit cell, i.e., to generate an experimental electron density map. This may be accomplished using programs of the CNX or CCP4 packages . The resolution is measured in Angstrom (Å) units, and is closely related to how far apart two objects need to be before they can be reliably distinguished. The smaller this number is, the higher the resolution and therefore the greater the amount of detail that can be seen. Preferably, crystals of the invention diffract x-rays to a resolution of better than about 4.0, 3.5, 3.0, 2.5, 2.0, 1.5, 1.0, 0.5 Å or better.

As used herein, the term “modeling” includes the quantitative and qualitative analysis of molecular structure and/or function based on atomic structural information and interaction models. The term “modeling” includes conventional numeric-based molecular dynamic and energy minimization models, interactive computer graphic models, modified molecular mechanics models, distance geometry and other structure-based constraint models.

Model building may be accomplished by either the crystallographer using a computer graphics program such as TURBO or O (Jones, T A. et al., Acta Crystallogr. A47, 100-119, 1991) or, under suitable circumstances, by using a fully automated model building program, such as wARP (Anastassis Perrakis, Richard Morris & Victor S. Lamzin; Nature Structural Biology, May 1999 Volume 6 Number 5 pp 458-463) or MAID (Levitt, D. G., Acta Crystallogr. D 2001 V57: 1013-9). This structure may be used to calculate model-derived diffraction amplitudes and phases. The model-derived and experimental diffraction amplitudes may be compared and the agreement between them can be described by a parameter referred to as R-factor. A high degree of correlation in the amplitudes corresponds to a low R-factor value, with 0.0 representing exact agreement and 0.59 representing a completely random structure. Because the R-factor may be lowered by introducing more free parameters into the model, an unbiased, cross-correlated version of the R-factor known as the R-free gives a more objective measure of model quality. For the calculation of this parameter a subset of reflections (generally around 10%) are set aside at the beginning of the refinement and not used as part of the refinement target. These reflections are then compared to those predicted by the model (Kleywegt G J, Brunger A T., Structure 1996 Aug 15;4(8):897-904).

The model may be improved using computer programs that maximize the probability that the observed data was produced from the predicted model, while simultaneously optimizing the model geometry. For example, the CNX program may be used for model refinement, as can the XPLOR program (1992, Nature 355:472-475, G. N. Murshudov, A. A. Vagin and E. J. Dodson, (1997) Acta Cryst. D 53, 240-255). In order to maximize the convergence radius of refinement, simulated annealing refinement using torsion angle dynamics may be employed in order to reduce the degrees of freedom of motion of the model (Adams P D, Pannu N S, Read R J, Brunger A T., Proc Natl Acad Sci U S A 1997 May 13;94(10):5018-23). Where experimental phase information is available (e.g. where MAD data was collected) Hendrickson-Lattman phase probability targets may be employed. Isotropic or anisotropic domain, group or individual temperature factor refinement, may be used to model variance of the atomic position from its mean. Well defined peaks of electron density not attributable to protein atoms are generally modeled as water molecules. Water molecules may be found by manual inspection of electron density maps, or with automatic water picking routines. Additional small molecules, including ions, cofactors, buffer molecules or substrates may be included in the model if sufficiently unambiguous electron density is observed in a map.

In general, the R-free is rarely as low as 0.15 and may be as high as 0.35 or greater for a reasonably well-determined protein structure. The residual difference is a consequence of approximations in the model (inadequate modeling of residual structure in the solvent, modeling atoms as isotropic Gaussian spheres, assuming all molecules are identical rather than having a set of discrete conformers, etc.) and errors in the data (Lattman E E., Proteins 1996; 25: i-ii). In refined structures at high resolution, there are usually no major errors in the orientation of individual residues, and the estimated errors in atomic positions are usually around 0.1-0.2 up to 0.3 Å, provided the amino acid sequence is known.

The three dimensional structure of a new crystal may be modeled using molecular replacement. The term “molecular replacement” refers to a method that involves generating a preliminary model of a molecule or complex whose structure coordinates are unknown, by orienting and positioning a molecule whose structure coordinates are known within the unit cell of the unknown crystal, so as best to account for the observed diffraction pattern of the unknown crystal. Phases may then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. Lattman, E., “Use of the Rotation and Translation Functions”, in Methods in Enzymology, 115, pp. 55-77 (1985); M. G. Rossmann, ed., “The Molecular Replacement Method”, Int. Sci. Rev. Ser., No. 13, Gordon & Breach, New York, (1972).

Commonly used computer software packages for molecular replacement are CNX, X-PLOR (Brunger 1992, Nature 355: 472-475), AMoRE (Navaza, 1994, Acta Crystallogr. A50:157-163), the CCP4 package, the MERLOT package (P. M. D. Fitzgerald, J. Appl. Cryst., Vol. 21, pp. 273-278, 1988) and XTALVIEW (McCree et al (1992) J. Mol. Graphics 10: 44-46). It is preferable that the resulting structure not exhibit a root-mean-square deviation of more than about 3 Å. The quality of the model may be analyzed using a program such as PROCHECK or 3D-Profiler [Laskowski et al 1993 J. Appl. Cryst. 26:283-291; Luthy R. et al, Nature 356: 83-85, 1992; and Bowie, J. U. et al, Science 253: 164-170, 1991].

Homology modeling (also known as comparative modeling or knowledge-based modeling) methods may also be used to develop a three dimensional model from a polypeptide sequence based on the structures of known proteins. The method utilizes a computer model of a known protein, a computer representation of the amino acid sequence of the polypeptide with an unknown structure, and standard computer representations of the structures of amino acids. This method is well known to those skilled in the art (Greer, 1985, Science 228, 1055; Bundell et al 1988, Eur. J. Biochem. 172, 513; Knighton et al., 1992, Science 258:130-135, http://biochem.vt.edu/courses/modeling/homology.htn). Computer programs that can be used in homology modeling are Quanta and the Homology module in the Insight II modeling package distributed by Molecular Simulations Inc, or MODELLER (Rockefeller University, www.iucr.ac.uk/sinris-top/logical/prg-modeller.html).

Once a homology model has been generated it is analyzed to determine its correctness. A computer program available to assist in this analysis is the Protein Health module in Quanta which provides a variety of tests. Other programs that provide structure analysis along with output include PROCHECK and 3D-Profiler [Luthy R. et al, Nature 356: 83-85, 1992; and Bowie, J. U. et al, Science 253: 164-170, 1991]. Once any irregularities have been resolved, the entire structure may be further refined.

Other molecular modeling techniques may also be employed in accordance with this invention. See, e.g., Cohen, N. C. et al, J. Med. Chem., 33, pp. 883-894 (1990). See also, Navix, M. A. and M. A. Marko, Current Opinions in Structural Biology, 2, pp. 202-210 (1992).

Under suitable circumstances, the entire process of solving a crystal structure may be accomplished in an automated fashion by a system such as ELVES (http://ucxray.berkeley.edu/˜jamesh/elves/index.html) with little or no user intervention.

A three dimensional structure of the molecule or complex may be described by the set of atoms that best predict the observed diffraction data (that is, which possesses a minimal R value). Files may be created for the structure that defines each atom by its chemical identity, spatial coordinates in three dimensions, root mean squared deviation from the mean observed position and fractional occupancy of the observed position. Hydrogen bonds and other atomic interactions, both within the protein and to bound ligands, can be identified with a high degree of confidence. A crystal structure of the present invention may be used to make a structural or computer model of the polypeptide. A model may represent the secondary, tertiary and/or quaternary structure of the polypeptide. The model itself may be in two or three dimensions.

Those of skill in the art understand that a set of structure coordinates for an protein, complex or a portion thereof, is a relative set of points that define a shape in three dimensions. Thus, it is possible that an entirely different set of coordinates could define a similar or identical shape. Moreover, slight variations in the individual coordinates may have little effect on overall shape. Such variations in coordinates may be generated because of mathematical manipulations of the structure coordinates. For example, structure coordinates could be manipulated by crystallographic permutations of the structure coordinates, fractionalization of the structure coordinates, integer additions or subtractions to sets of the structure coordinates, inversion of the structure coordinates or any combination of the above.

Alternatively, modifications in the crystal structure due to mutations, additions, substitutions, and/or deletions of amino acids, or other changes in any of the components that make up the crystal could also account for variations in structure coordinates. If such variations are within an acceptable standard error as compared to the original coordinates, the resulting three-dimensional shape is considered to be the same.

For the purpose of this invention, any molecule, protein, complex or fragment or portion thereof that has a root mean square deviation of conserved residue backbone atoms (e.g., for a polypeptide, N, Cα, C, O) of less than 1.75 Å when superimposed on the relevant backbone atoms described by structure coordinates of a related material are considered identical. Alternatively, the root mean square deviation is less than about 1.50, 1.25 or 1.0 Å. The term “root mean square deviation” means the square root of the arithmetic mean of the squares of the deviations from the mean. It is a way to express the deviation or variation from a trend or object. For purposes of this invention, when used in reference to a polypeptide, the “root mean square deviation” defines the variation in the backbone of a protein from the backbone of another protein, such as a polypeptide or a fragment or portion thereof.

In another embodiment, a computer may be used to produce a three-dimensional representation of a polypeptide, or a complex containing said polypeptide, defined by structure coordinates, or a three-dimensional representation of a homologue of said molecule or complex, wherein said homologue comprises a amino acid sequence that has a root mean square deviation from the backbone atoms of the amino acids of said polypeptide of not more than 1.5 Å.

According to an alternate embodiment, the invention provides a computer for determining at least a portion of the structure coordinates corresponding to X-ray diffraction data obtained from a molecule or molecular complex, wherein said computer comprises:

(a) a machine-readable data storage medium comprising a data storage material encoded with machine-readable data, wherein said data comprises at least a portion of the structural coordinates of a polypeptide;

(b) a machine-readable data storage medium comprising a data storage material encoded with machine-readable data, wherein said data comprises X-ray diffraction data from said molecule or molecular complex;

(c) a working memory for storing instructions for processing said machine-readable data of (a) and (b);

(d) a central-processing unit coupled to said working memory and to said machine-readable data storage medium of (a) and (b) for performing a Fourier transform of the machine readable data of (a) and for processing said machine readable data of (b) into structure coordinates; and

(e) a display coupled to said central-processing unit for displaying said structure coordinates of said molecule or molecular complex.

For example, the Fourier transform of the structure coordinates of a polypeptide may be used to determine at least a portion of the structure coordinates of other related polypeptides.

Thus, in accordance with the present invention, X-ray coordinate data capable of being processed into a three dimensional graphical display of a polypeptide or a fragment or complex thereof. The X-ray coordinate data, when used in conjunction with a computer programmed with software to translate those coordinates into the 3-dimensional structure of a molecule or molecular complex, may be used for a variety of purposes, such as drug discovery, as described in greater detail below. For example, the structure encoded by the data may be computationally evaluated for its ability to associate with chemical entities. Chemical entities that associate with a polypeptide, or a portion thereof, and thereby inhibit that enzyme are potential drug candidates. Alternatively, the structure encoded by the data may be displayed in a graphical three-dimensional representation on a computer screen. This allows visual inspection of the structure, as well as visual inspection of the structure's association with chemical entities.

In another embodiment, the structural coordinates of a known crystal structure may be applied to nuclear magnetic resonance (NMR) data to determine the three dimensional structures of polypeptides with uncharacterized or incompletely characterized structure. (See for example, Wuthrich, 1986, John Wiley and Sons, New York: 176-199; Pflugrath et al., 1986, J. Molecular Biology 189: 383-386; Kline et al., 1986 J. Molecular Biology 189:377-382). While the secondary structure of a polypeptide may often be determined by NMR data, the spatial connections between individual pieces of secondary structure are not as readily determined. The structural coordinates of a polypeptide defined by X-ray crystallography can guide the NMR spectroscopist to an understanding of the spatial interactions between secondary structural elements in a polypeptide of related structure. Information on spatial interactions between secondary structural elements can greatly simplify Nuclear Overhauser Effect (NOE) data from two-dimensional NMR experiments. In addition, applying the structural coordinates after the determination of secondary structure by NMR techniques simplifies the assignment of NOE's relating to particular amino acids in the polypeptide sequence and does not greatly bias the NMR analysis of polypeptide structure.

In an embodiment, the invention relates to a method of determining three dimensional structures of polypeptides with unknown structures, by applying the structural coordinates of a crystal of the present invention to nuclear magnetic resonance (NMR) data of the unknown structure. This method comprises the steps of: (a) determining the secondary structure of an unknown structure using NMR data; and (b) simplifying the assignment of through-space interactions of amino acids. The term “through-space interactions” defines the orientation of the secondary structural elements in the three dimensional structure and the distances between amino acids from different portions of the amino acid sequence. The term “assignment” defines a method of analyzing NMR data and identifying which amino acids give rise to signals in the NMR spectrum.

See also Brooks et al. (1983) J Comput Chem 4:187-217; Weiner et al (1981) J. Comput. Chem. 106: 765; Eisenfield et al. (1991) Am J Physiol 261:C376-386; Lybrand (1991) J Pharm Belg 46:49-54; Froimowitz (1990) Biotechniques 8:640-644; Burbam et al. (1990) Proteins 7:99-111; Pedersen (1985) Environ Health Perspect 61:185-190; and Kini et al. (1991) J Biomol Struct Dyn 9:475-488; Ryckaert et al. (1977) J Comput Phys 23:327; Van Gunsteren et al. (1977) Mol Phys 34:1311; Anderson (1983) J Comput Phys 52:24; J. Mol. Biol. 48: 442-453, 1970; Dayhoff et al., Meth. Enzymol. 91: 524-545, 1983; Henikoff and Henikoff, Proc. Nat. Acad. Sci. USA 89: 10915-10919, 1992; J. Mol. Biol. 233: 716-738, 1993; Methods in Enzymology, Volume 276, Macromolecular crystallography, Part A, ISBN 0-12-182177-3 and Volume 277, Macromolecular crystallography, Part B, ISBN 0-12-182178-1, Eds. Charles W. Carter, Jr. and Robert M. Sweet (1997), Academic Press, San Diego; Pfuetzner, et al., J. Biol. Chem. 272:430-434 (1997).

5. Rational Drug Design and Structure Guided Drug Design

Once the three-dimensional structure of a polypeptide is determined by the methods disclosed herein, a potential modulator (drug, agent, test compound, etc.) may be examined either through visual inspection or through the use of computer modeling using a docking program such as GRAM, DOCK, or AUTODOCK (Dunbrack et al., Folding & Design, 2:27-42 (1997)). This procedure can include computer fitting of potential drugs to a particular macromolecule to ascertain how well the shape and the chemical structure of the potential ligand will complement or interfere with the structure of the subject polypeptide (Bugg et al., Scientific American, Dec.: 92-98 (1993); West et al., TIPS, 16:67-74 (1995)). Computer programs may also be employed to estimate the attraction, repulsion, and steric hindrance of the potential drug to a binding site, for example. Generally, the tighter the fit (e.g., the lower the steric hindrance, and/or the greater the attractive force) the more potent the potential drug will be because these properties are consistent with a tighter binding constant. Furthermore, the more specificity in the design of a potential drug the more likely that the drug will not interfere with related proteins, which may minimize potential side-effects due to unwanted interactions.

The increasing availability of biomacromolecule structures that have been solved crystallographically has prompted the development of a variety of direct computational methods for molecular design, in which the steric and electronic properties of druggable target sites are use to guide the design of potential agents (Cohen et al. (1990) J. Med. Cam. 33: 883-894; Kuntz et al. (1982) J. Mol. Biol 161: 269-288; DesJarlais (1988) J. Med. Cam. 31: 722-729; Bartlett et al. (1989) (Spec. Publ., Roy. Soc. Chem.) 78: 182-196; Goodford et al. (1985) J. Med. Cam. 28: 849-857; DesJarlais et al. J. Med. Cam. 29: 2149-2153). Directed methods generally fall into two categories: (1) design by analogy in which 3-D structures of known molecules (such as from a crystallographic database) are docked to the polypeptide structure and scored for goodness-of-fit; and (2) de novo design, in which the test compound model is constructed piece-wise in the druggable target site. The test compound may be screened as part of a library or a data base of molecules. Data bases which may be used include ACD (Molecular Designs Limited), NCI (National Cancer Institute), CCDC (Cambridge Crystallographic Data Center), CAST (Chemical Abstract Service), Derwent (Derwent Information Limited), Maybridge (Maybridge Chemical Company Ltd), Aldrich (Aldrich Chemical Company), DOCK (University of California in San Francisco), and the Directory of Natural Products (Chapman & Hall). Computer programs such as CONCORD (Tripos Associates) or DB-Converter (Molecular Simulations Limited) can be used to convert a data set represented in two dimensions to one represented in three dimensions. In addition, structural information on the subject polypeptides may be used.

Test compounds may be tested for their capacity to fit spatially into a druggable target site. As used herein, the term “fits spatially” means that the three-dimensional structure of the test compound is accommodated geometrically in a cavity of the druggable site. The test compound may then be considered to be a drug candidate. A favorable geometric fit occurs when the surface area of the test compound is in close proximity with the surface area of the cavity of a druggable site without forming unfavorable interactions. A favorable complementary interaction occurs where the test compound interacts by hydrophobic, aromatic, ionic, dipolar, or hydrogen donating and accepting forces. Unfavorable interactions may be steric hindrance between atoms in the test compound and atoms in the druggable site.

If a model of the present invention is a computer model, the test compounds may be positioned in a druggable site through computational docking. If, on the other hand, the model of the present invention is a structural model, the test compounds may be positioned in the druggable site by, for example, manual docking. As used herein the term “docking” refers to a process of placing a compound in close proximity with a druggable site, or a process of finding low energy conformations of a test compound/druggable site complex.

In an illustrative embodiment, the design of potential drug candidates begins from the general perspective of shape complimentary for the druggable site of a polypeptide, and a search algorithm is employed which is capable of scanning a database of small molecules of known three-dimensional structure for candidates which fit geometrically into the target druggable site. Most algorithms of this type provide a method for finding a wide assortment of chemical structures that are complementary to the shape of a druggable target of the subject polypeptide. Each of a set of small molecules from a particular data-base, such as the Cambridge Crystallographic Data Bank (CCDB) (Allen et al. (1973) J. Chem. Doc. 13: 119), is individually docked to the druggable target site of a polypeptide in a number of geometrically permissible orientations with use of a docking algorithm. In certain embodiments, a set of computer algorithms called DOCK, can be used to characterize the shape of invaginations and grooves that form the active sites and recognition surfaces of the subject polypeptide (Kuntz et al. (1982) J. Mol. Biol 161: 269-288). The program can also search a database of small molecules for templates whose shapes are complementary to particular binding sites of a polypeptide (DesJarlais et al. (1988) J Med Chem 31: 722-729).

The orientations are evaluated for goodness-of-fit and the best are kept for further examination using molecular mechanics programs, such as AMBER or CHARMM. Such algorithms have previously proven successful in finding a variety of molecules that are complementary in shape to a given druggable site of a polypeptide, and have been shown to have several attractive features.

Goodford (1985, J Med Chem 28:849-857) and Boobbyer et al. (1989, J Med Chem 32:1083-1094) have produced a computer program (GRID) which seeks to determine regions of high affinity for different chemical groups (termed probes) on the molecular surface of the binding site. GRID hence provides a tool for suggesting modifications to known ligands that might enhance binding. It may be anticipated that some of the sites discerned by GRID as regions of high affinity correspond to “pharmacophoric patterns” determined inferentially from a series of known ligands. As used herein, a “pharmacophoric pattern” is a geometric arrangement of features of the anticipated ligand that is believed to be important for binding. Attempts have been made to use pharmacophoric patterns as a search screen for novel ligands (Jakes et al. (1987) J Mol Graph 5:41-48; Brint et al. (1987) J Mol Graph 5:49-56; Jakes et al. (1986) J Mol Graph 4:12-20).

Yet a further embodiment of the present invention utilizes a computer algorithm such as CLIX which searches such databases as CCDB for small molecules which can be oriented in the receptor binding site in a way that is both sterically acceptable and has a high likelihood of achieving favorable chemical interactions between the candidate molecule and the surrounding amino acid residues. The method is based on characterizing the receptor site in terms of an ensemble of favorable binding positions for different chemical groups and then searching for orientations of the candidate molecules that cause maximum spatial coincidence of individual candidate chemical groups with members of the ensemble. The algorithmic details of CLIX is described in Lawrence et al. (1992) Proteins 12:31-41.

In one instance, a potential drug could be obtained by screening a peptide library (Scott and Smith, Science, 249:386-390 (1990); Cwirla et al., Proc. Natl. Acad. Sci., 87:6378-6382 (1990); Devlin et al., Science, 249:404-406 (1990)). A potential drug selected in this manner could be then be systematically modified by computer modeling programs until one or more promising potential drugs are identified. Such analysis has been shown to be effective in the development of HIV protease inhibitors (Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1:109-128 (1993)).

Alternatively a potential modulator may be selected from a library of chemicals such as those that can be licensed from third parties, such as chemical and pharmaceutical companies. A third alternative is to synthesize the potential drug de novo.

A number of techniques may be used to design, evaluate and otherwise characterize compounds using structural information about the target in a process known as structure guided drug design. Computational techniques can be used to screen, identify, select and design chemical entities capable of associating with a molecule or complex, e.g., protein or protein complex. Knowledge of the structure coordinates of a molecule or complex permits the design and/or identification of synthetic compounds and/or other molecules which have a shape complementary to the conformation of a binding site of the molecule or complex. In particular, computational techniques can be used to identify or design chemical entities, such as inhibitors, agonists and antagonists, that associate with a binding pocket. Inhibitors may bind to or interfere with all or a portion of a binding pocket, and can be competitive, non-competitive, or uncompetitive inhibitors; or interfere with dimerization by binding at the interface between the two monomers. Once identified and screened for biological activity, these inhibitors/agonists/antagonists may be used therapeutically or prophylactically to block activity of the molecule or complex and. Structure-activity data for analogs of ligands that bind to or interfere with binding pockets can also be obtained computationally.

The term “chemical entity,” as used herein, refers to agents, complexes of two or more agents, and fragments of such agents or complexes. Chemical entities that are determined to associate with a molecule or complex are potential drug candidates. Data stored in a machine-readable storage medium that is capable of displaying a graphical three-dimensional representation of the structure of a molecule or complex, as identified herein, or portions thereof may thus be advantageously used for drug discovery. The structure coordinates of the chemical entity are used to generate a three-dimensional image that can be computationally fit to the three-dimensional image of the molecule or complex or portion thereof. The three-dimensional molecular structure encoded by the data in the data storage medium can then be computationally evaluated for its ability to associate with chemical entities. When the molecular structures encoded by the data is displayed in a graphical three-dimensional representation on a computer screen, the protein structure can also be visually inspected for potential association with chemical entities.

The chemical entities and compounds used in the present invention may de described in a number of ways. Some illustrative and non-limiting examples include the following. For example, chemical entities and compounds may contain one or more aromatic substructures, with one or more rings. Alternatively, the ring structures may not be aromatic in nature. In another aspect, the chemical entities and compounds may be characterized as having at least a certain number of carbon atoms, such as at least about 6, 10, 20 or alternatively from about 10 to 50 carbon atoms, etc. In yet another aspect, the chemical entities and compounds may contain certain atoms and chemical moieties, such as carbon-fluorine bonds, which are usually non-reactive at physiological conditions. The various means of describing the chemical entities and compounds may be combined, e.g., a chemical entity or compound of the present inventions includes at least about six carbon atoms, two fluorine atoms, two ring structures, optionally aromatic. Other combinations like that one are known to those of skill in the art, as are other ways of describing the chemical entities and compounds of the present invention.

One embodiment of the method of drug design involves evaluating the potential association of a known chemical entity with a molecule or complex, e.g., with a binding pocket. The method of drug design thus includes computationally evaluating the potential of a selected chemical entity to associate with any of the molecules or molecular complexes set forth above. This method may comprise the steps of: (a) employing computational means to perform a fitting operation between the selected chemical entity and a site of interest, e.g., a binding pocket, of the molecule or molecular complex; and (b) analyzing the results of said fitting operation to quantify the association between the chemical entity and the site of interest.

In another embodiment, the method of drug design involves computer assisted design of chemical entities that associate with a molecule or complex or portions thereof. Chemical entities can be designed in a step-wise fashion, one fragment at a time, or may be designed as a whole or “de novo.” To be a viable drug candidate, the chemical entity identified or designed according to the method must be capable of structurally associating with at least part of a site of interest on the molecule or complex, and must be able, sterically and energetically, to assume a conformation that allows it to associate with the molecule or complex. Non-covalent molecular interactions important in this association include hydrogen bonding, van der Waals interactions, hydrophobic interactions, and electrostatic interactions. Conformational considerations include the overall three-dimensional structure and orientation of the chemical entity in relation to the site of interest, e.g., binding pocket, and the spacing between various functional groups of an entity that directly interact with the site of interest on the molecule or complex.

Optionally, the potential binding of a chemical entity to a site of interest is analyzed using computer modeling techniques prior to the actual synthesis and testing of the chemical entity. If these computational experiments suggest insufficient interaction and association between it and the site of interest on the molecule or complex, testing of the entity is obviated. However, if computer modeling indicates a strong interaction, the molecule may then be synthesized and tested for its ability to bind to or interfere with the site of interest on the molecule or complex. Binding assays to determine if a compound actually binds to the site of interest can also be performed and are well known in the art. Binding assays may employ kinetic or thermodynamic methodology using a wide variety of techniques including, but not limited to, microcalorimetry, circular dichroism, capillary zone electrophoresis, nuclear magnetic resonance spectroscopy, fluorescence spectroscopy, and combinations thereof.

One skilled in the art may use one of several methods to screen chemical entities or fragments for their ability to associate with a site of interest on the molecule or complex, e.g., a binding pocket. This process may begin by visual inspection of, for example, the molecule or complex or particular portion thereof on the computer screen based on the structure coordinates of the molecule or complex or portion thereof or other coordinates which define a similar shape generated from the machine-readable storage medium. Selected fragments or chemical entities may then be positioned in a variety of orientations, or docked, within the binding pocket. Docking may be accomplished using software such as QUANTA and SYBYL, followed by energy minimization and molecular dynamics with standard molecular mechanics forcefields, such as CHARMM and AMBER.

Specialized computer programs may also assist in the process of selecting fragments or chemical entities. Examples include GRID (P. J. Goodford, J. Med. Chem. 28:849-857 (1985); available from Oxford University, Oxford, UK); MCSS (A. Miranker et al., Proteins: Struct. Funct. Genj 1:29-34 (1991); available from Molecular Simulations, San Diego, Calif.); AUTODOCK (D. S. Goodsell et al., Proteins: Struct. Funct. Genet. 8:195-202 (1990); available from Scripps Research Institute, La Jolla, Calif.); and DOCK (I. D. Kuntz et al., J. Mol. Biol. 161:269-288 (1982); available from University of California, San Francisco, Calif.).

Once suitable chemical entities or fragments have been selected, they can be assembled into a single compound or complex. Assembly may be preceded by visual inspection of the relationship of the fragments to each other on the three dimensional image displayed on a computer screen in relation to the structure coordinates of the molecule or complex or portion thereof. This can be followed by manual model building using software such as QUANTA or SYBYL (Tripos Associates, St. Louis, Mo.).

Useful programs to aid one of skill in the art in connecting the individual chemical entities or fragments include, without limitation, CAVEAT (P. A. Bartlett et al., in Molecular Recognition in Chemical and Biological Problems,” Special Publ., Royal Chem. Soc., 78:182-196 (1989); G. Lauri et al., J. Comput. Aided Mol. Des. 8:51-66 (1994); available from the University of California, Berkeley, Calif.); 3D database systems such as ISIS (available from MDL Information Systems, San Leandro, Calif.; reviewed in Y. C. Martin, J. Med. Chem. 35:2145-2154 (1992)); and HOOK (M. B. Eisen et al., Proteins: Struc., Funct., Genet. 19:199-221 (1994); available from Molecular Simulations, San Diego, Calif.).

Compounds binding to a particular site on a molecule or complex may be designed “de novo” using either an empty binding site or optionally including some portion(s) of a known inhibitor(s). There are many de novo ligand design methods including, without limitation, LUDI (H. -J. Bohm, J. CoMp. Aid. Molec. Design. 6:61-78 (1992); available from Molecular Simulations Inc., San Diego, Calif.); LEGEND (Y. Nishibata et al., Tetrahedron, 47:8985 (1991); available from Molecular Simulations Inc., San Diego, Calif.); LeapFrog (available from Tripos Associates, St. Louis, Mo.); and SPROUT (V. Gillet et al., J. Cpmput. Aided Mol. Desi 7:127-153 (1993); available from the University of Leeds, UK).

Once a compound has been designed or selected by the above methods, the efficiency with which that entity may bind to or interfere with a molecule or molecular complex may be tested and optimized by computational evaluation. For example, an effective inhibitor must preferably demonstrate a relatively small difference in energy between its bound and free states (i.e., a small deformation energy of binding). Thus, the most efficient inhibitors should preferably be designed with a deformation energy of binding of not greater than about 10 kcal/mole; more preferably, not greater than 7 kcal/mole. Inhibitors may interact with the binding pocket in more than one conformation that is similar in overall binding energy. In those cases, the deformation energy of binding is taken to be the difference between the energy of the free entity and the average energy of the conformations observed when the inhibitor binds to the protein.

An entity designed or selected as binding to or interfering with a molecule or complex may be further computationally optimized so that in its bound state it would preferably lack repulsive electrostatic interaction with the target enzyme and with the surrounding water molecules. Such non-complementary electrostatic interactions include repulsive charge-charge, dipole-dipole, and charge-dipole interactions.

Specific computer software is available in the art to evaluate compound deformation energy and electrostatic interactions. Examples of programs designed for such uses include: Gaussian 94, revision C (M. J. Frisch, Gaussian, Inc., Pittsburgh, Pa. (1995)); AMBER, version 4.1 (P. A. Kollman, University of California at San Francisco, (1995)); QUANTA/CHARI 4M (Molecular Simulations, Inc., San Diego, Calif. (1995)); Insight II/Discover (Molecular Simulations, Inc., San Diego, Calif. (1995)); DelPhi (Molecular Simulations, Inc., San Diego, Calif. (1995)); and AMSOL (Quantum Chemistry Program Exchange, Indiana University). These programs may be implemented, for instance, using a Silicon Graphics workstation such as an Indigo2 with “MPACT” graphics. Other hardware systems and software packages will be known to those skilled in the art.

Another approach encompassed by this invention is the computational screening of databases for small molecules, chemical entities, compounds or other modulators that can bind in whole, or in part, to a molecule or complex. In this screening, the quality of fit of such entities to the binding site may be judged either by shape complementarity or by estimated interaction energy (E. C. Meng et al., J. Comp. Chem., 13, pp. 505-524 (1992)).

This invention also enables the development of chemical entities that can isomerize to short-lived reaction intermediates in the chemical reaction of a substrate or other compound that binds to or with a molecule or complex. Time-dependent analysis of structural changes in the molecule or complex during its interaction with other molecules is carried out. The reaction intermediates of the molecule or complex can also be deduced from the reaction product in co-complex with the molecule or complex. Such information is useful to design improved analogs of know inhibitors or to design novel classes of inhibitors based on the reaction intermediates of the molecule or complex and inhibitor co-complex. This provides a novel route for designing inhibitors with both high specificity and stability.

Yet another approach to rational drug design involves probing the molecule or complex crystalwith molecules comprising a variety of different functional groups to determine optimal sites for interaction between candidate inhibitors and the protein. For example, high resolution x-ray diffraction data collected from crystals soaked in or co-crystallized with other molecules allows the determination of where each type of solvent molecule sticks. Molecules that bind tightly to those sites can then be further modified and synthesized and tested for their hepes protease inhibitor activity (J. Travis, Science, 262:1374 (1993)).

In a related approach, iterative drug design can be used to identify inhibitors of a molecule or complex. Iterative drug design is a method for optimizing associations between a protein and a compound by determining and evaluating the three dimensional structures of successive sets of protein/compound complexes. In iterative drug design, crystals of a series of protein/compound complexes are obtained and then the three-dimensional structures of each complex is solved. Such an approach provides insight into the association between the proteins and compounds of each complex. This is accomplished by selecting compounds with inhibitory activity, obtaining crystals of this new protein/compound complex, solving the three-dimensional structure of the complex, and comparing the associations between the new protein/compound complex and previously solved protein/compound complexes. By observing how changes in the compound affected the protein/compound associations, these associations may be optimized.

The structural analysis disclosed herein in conjunction with computer modeling allows the selection of a finite number of rational chemical modifications, as opposed to the countless number of essentially random chemical modifications that could be made, any of which might lead to a useful drug. Each chemical modification requires additional chemical steps, which while being reasonable for the synthesis of a finite number of compounds, quickly becomes overwhelming if all possible modifications needed to be synthesized. Thus through the use of the methodology disclosed herein and computer modeling, a large number of these compounds can be rapidly screened on the computer monitor screen, and a few likely candidates can be determined without the laborious synthesis of untold numbers of compounds. As mentioned above, the de novo synthesis of one or even a relatively small group of specific compounds is reasonable in the art of drug design.

Once a potential modulator is identified, it can then be tested in any standard assay for the macromolecule depending of course on the macromolecule, including in high throughput assays. When a suitable potential drug is identified, a further NMR structural analysis may optionally be performed.

For all of the drug screening assays described herein further refinements to the structure of the drug will generally be necessary and can be made by the successive iterations of any and/or all of the steps provided by the particular drug screening assay, in particular further structural analysis by e.g., ¹⁵N NMR relaxation rate determinations or x-ray crystallography with the modulator bound to the subject polypeptide. These studies may be performed in conjunction with biochemical assays, which are described above in part are well known to the skilled artisan.

Once identified, a potential drug candidate may be used as a model structure, and analogs to the compound can be obtained (e.g., from the vast chemical libraries that can be licensed for the large chemical companies as cited above, or alternatively through de novo synthesis). The analogs are then screened for their ability to bind the subject polypeptide. An analog of the potential drug candidate might be chosen as a drug candidate when it binds to the subject polypeptide with a higher binding affinity than the potential drug candidate.

In another embodiment, compounds are screened for binding to two nearby sites on polypeptide. In this case, a compound that binds a first site of the subject polypeptide does not bind a second nearby site. Binding to the second site can be determined by monitoring changes in a different set of amide chemical shifts in either the original screen or a second screen conducted in the presence of a drug candidate (or potential drug candidate) for the first site. From an analysis of the chemical shift changes the approximate location of a potential drug candidate for the second site is identified. Optimization of the second drug candidate for binding to the site is then carried out by screening structurally related compounds (e.g., analogs as described above). When drug candidates for the first site and the second site are identified, their location and orientation in the ternary complex can be determined experimentally either by standard NMR spectroscopy, an/or X-ray crystallography. On the basis of this structural information, a linked compound, e.g., a consolidated drug candidate, is synthesized in which the drug candidate for the first site and the drug candidate for the second site are linked. In certain embodiments, the two drug candidates are covalently linked to form a consolidated drug candidate. This consolidated drug candidate may be tested to determine if it has a higher binding affinity for the macromolecule than either of the two individual drug candidates. A consolidated drug candidate is selected as a drug candidate when it has a higher binding affinity for the macromolecule than either of the two drug candidates. Larger consolidated drug candidates can be constructed in an analogous manner, e.g., linking three drug candidates which bind to three nearby sites on the macromolecule to form a multilinked consolidated drug candidate that has an even higher affinity for the macromolecule than linked compound.

In still another aspect of the present invention, solution and/or crystal structures of individual domains of a multidomain protein can first be determined and then used as high resolution structures for the procedure of defining relative domain orientation disclosed herein for the intact multidomain protein. The resulting structural determination for the multidomain protein can then be used as to identify new binding sites arising from the close interactions of the constituent domains. The binding sites that are identified can in turn be used as a target for rational drug design in order to identify bioactive compounds useful as therapeutic agents (e.g. drugs) or alternatively as diagnostic reagents of the state of the protein. Such changes in relative orientation of protein domains might occur as the result of postsynthetic modifications, e.g., protein phosphorylation in which a tyrosine, serine, histidine, or threonine residue is phosphorylated (Sicheri and Kuriyan, Curr, Op, Str. Biol. 7: 777-785 (1997)).

The methods provided by the present invention may also be used in designing new polypeptides to aid in drug discovery. Thus based on analysis of the relative orientations of the components by the methods disclosed herein, novel polypeptides may be constructed through either total synthesis or by ligation of expressed proteins of chimeras, whose individual component structures can be precisely modified by site specific mutation (or site directed substitution), or residue or component substitution by total synthesis.

The present invention further provides a method of using NMR in combination with a high resolution crystal structure of a multidomain protein to define the likely orientation of heteronuclear bonds in component domains, as described above. In this case NMR would be used to define the actual, in solution, component orientations. This is likely to differ from the crystal structure form, and thereby provide unique information for rational drug design as outlined above.

6. Activity and Other Assays

In certain embodiments, the methods of the invention may utilize an activity assay to monitor the function of a polypeptide, characterize the ability of a molecule to bind to a polypeptide, and/or characterize the ability of a molecule to modify the activity of a polypeptide. Both in vitro and in vivo assays may be used in accordance with the methods of the invention depending on the identity of the polypeptide being investigated. Appropriate activity or functional assays may be readily determined by the skilled artisan based on the disclosure herein.

The activity of a polypeptide may be identified and/or assayed using a variety of methods well known to the skilled artisan. For example, information about the activity of non-essential genes may be assayed by creating a null mutant strain of bacteria expressing a mutant form of, or lacking expression of, a protein of interest. The resulting phenotype of the null mutant strain may provide information about the activity of the mutated gene product. Essential genes may be studied by creating a bacterial strain with a conditional mutation in the gene of interest. The bacterial strain may be grown under permissive and non-permissive conditions and the change in phenotype under the non-permissive conditions may be used to identify and/or assay the activity of the gene product.

In an alternative embodiment, the activity of a protein may be assayed using an appropriate substrate or binding partner or other reagent suitable to test for the suspected activity. For catalytic activity, the assay is typically designed so that the enzymatic reaction produces a detectable signal. For example, mixture of a kinase with a substrate in the presence of ³²P will result in incorporation of the ³²P into the substrate. The labeled substrate may then be separated from the free ³²P and the presence and/or amount of radiolabeled substrate may be detected using a scintillation counter or a phosphorimager. Similar assays may be designed to identify and/or assay the activity of a wide variety of enzymatic activities. Based on the teachings herein, the skilled artisan would readily be able to develop an appropriate assay for a polypeptide.

In another embodiment, the activity of a polypeptide may be determined by assaying for the level of expression of RNA and/or protein molecules. Transcription levels may be determined, for example, using Northern blots, hybridization to an oligonucleotide array or by assaying for the level of a resulting protein product. Translation levels may be determined, for example, using Western blotting or by identifying a detectable signal produced by a protein product (e.g., fluorescence, luminescence, enzymatic activity, etc.). Depending on the particular situation, it may be desirable to detect the level of transcription and/or translation of a single gene or of multiple genes.

Alternatively, it may be desirable to measure the overall rate of DNA replication, transcription and/or translation in a cell. In general this may be accomplished by growing the cell in the presence of a detectable metabolite which is incorporated into the resultant DNA, RNA, or protein product. For example, the rate of DNA synthesis may be determined by growing cells in the presence of BrdU which is incorporated into the newly synthesized DNA. The amount of BrdU may then be determined histochemically using an anti-BrdU antibody.

In certain embodiments of the subject method, it may be advantageous to assess the activity of small molecules and other moieties in in vitro assays. In one embodiment of such an assay, agents are identified which modulate the biological activity of a protein, protein-protein interaction of interest or protein complex, such as an enzymatic activity, binding to other cellular components, cellular compartmentalization, signal transduction, and the like. In certain embodiments, the test agent is a small organic molecule.

The invention also provides a method of screening compounds to identify those which modulate the action of a polypeptide. The method of screening may involve high-throughput techniques. For example, to screen for modulators, a synthetic reaction mix, a cellular compartment, such as a membrane, cell envelope or cell wall, or a preparation of any thereof, comprising a polypeptide and a labeled substrate or ligand of such polypeptide is incubated in the absence or the presence of a candidate molecule that may be a modulator of a polypeptide. The ability of the candidate molecule to modulate a polypeptide is reflected in decreased binding of the labeled ligand or decreased production of product from such substrate. Detection of the rate or level of production of product from substrate may be enhanced by using a reporter system. Reporter systems that may be useful in this regard include but are not limited to colorimetric labeled substrate converted into product, a reporter gene that is responsive to changes in a polynucleotide of the invention or polypeptide activity, and binding assays known in the art.

Another example of an assay for a modulator of a polypeptide that may be used in accordance with the methods of the invention is a competitive assay that combines a polypeptide and a potential modulator with molecules that bind to a polypeptide, recombinant molecules that bind to a polypeptide, natural substrates or ligands, or substrate or ligand mimetics, under appropriate conditions for a competitive inhibition assay. Polypeptides can be labeled, such as by radioactivity or a colorimetric compound, such that the number of molecules of a polypeptide bound to a binding molecule or converted to product can be determined accurately to assess the effectiveness of the potential modulator.

Potential antagonists include small molecules, peptides, polypeptides and antibodies that bind to a polynucleotide or polypeptide and thereby inhibit or extinguish its activity. Potential antagonists also may be small molecules, a peptide, a polypeptide such as a closely related protein or antibody that bind the same sites on a binding molecule without inducing the activity normally induced by a polypeptide, thereby preventing the action of a polypeptide by excluding the polypeptide from binding. Potential antagonists include a small molecule that binds to and occupies the binding site of the polypeptide thereby preventing binding to cellular binding molecules, such that normal biological activity is prevented.

The polynucleotides of the invention may be used in the discovery and development of antibacterial compounds and other therapeutics and drugs. The encoded protein, upon expression, can be used as a target for the screening of drugs. Additionally, the DNA sequences encoding the amino terminal regions of the encoded protein or Shine-Delgamo or other translation facilitating sequences of the respective mRNA can be used to construct antisense sequences to control the expression of the coding sequence of interest.

A number of in vivo assays are contemplated by the present invention. For example, Animal models of bacterial infection and/or other diseases and conditions may be used as an in vivo assay for evaluating the effectiveness of a protein or site. A number of suitable animal models are described briefly below, however, these models are only examples and modifications, or completely different animal models, may be used in accord with the methods of the invention.

(i) Mouse Soft Tissue Model

The mouse soft tissue infection model is a sensitive and effective method for measurement of bacterial proliferation. In these models (Vogelman et al., 1988, J. Infect. Dis. 157: 287-298) anesthetized mice are infected with the bacteria in the muscle of the hind thigh. The mice can be either chemically immune compromised (e.g., cytoxan treated at 125 mg/kg on days −4, −2, and 0) or immunocompetent. The dose of microbe necessary to cause an infection is variable and depends on the individual microbe, but commonly is on the order of 10⁵-10⁶ colony forming units per injection for bacteria. A variety of mouse strains are useful in this model although Swiss Webster and DBA2 lines are most commonly used. Once infected the animals are conscious and show no overt ill effects of the infections for approximately 12 hours. After that time virulent strains cause swelling of the thigh muscle, and the animals can become bacteremic within approximately 24 hours. This model most effectively measures proliferation of the microbe, and this proliferation is measured by sacrifice of the infected animal and counting colonies from homogenized thighs.

(ii) Diffusion Chamber Model

A second model useful for assessing the virulence of microbes is the diffusion chamber model (Malouin et al., 1990, Infect. Immun. 58: 1247-1253; Doy et al., 1980, J. Infect. Dis. 2: 39-51; Kelly et al., 1989, Infect. Immun. 57: 344-350. In this model rodents have a diffusion chamber surgically placed in the peritoneal cavity. The chamber consists of a polypropylene cylinder with semipermeable membranes covering the chamber ends. Diffusion of peritoneal fluid into and out of the chamber provides nutrients for the microbes. The progression of the “infection” may be followed by examining growth, the exoproduct production or RNA messages. The time experiments are done by sampling multiple chambers.

(iii) Endocarditis Model

For bacteria, an important animal model effective in assessing pathogenicity and virulence is the endocarditis model (J. Santoro and M. E. Levinson, 1978, Infect. Immun. 19: 915-918). A rat endocarditis model can be used to assess colonization, virulence and proliferation.

(iv) Osteomyelitis Model

A fourth model useful in the evaluation of pathogenesis is the osteomyelitis model (Spagnolo et al., 1993, Infect. Immun. 61: 5225-5230). Rabbits are used for these experiments. Anesthetized animals have a small segment of the tibia removed and microorganisms are microinjected into the wound. The excised bone segment is replaced and the progression of the disease is monitored. Clinical signs, particularly inflammation and swelling are monitored. Termination of the experiment allows histolic and pathologic examination of the infection site to complement the assessment procedure.

(v) Murine Septic Arthritis Model

A fifth model relevant to the study of microbial pathogenesis is a murine septic arthritis model (Abdelnour et al., 1993, Infect. Immun. 61: 3879-3885). In this model mice are infected intravenously and pathogenic organisms are found to cause inflammation in distal limb joints. Monitoring of the inflammation and comparison of inflammation vs. inocula allows assessment of the virulence of related strains.

(vi) Bacterial Peritonitis Model

Finally, bacterial peritonitis offers rapid and predictive data on the virulence of strains (M. G. Bergeron, 1978, Scand. J. Infect. Dis. Suppl. 14: 189-206; S. D. Davis, 1975, Antimicrob. Agents Chemother. 8: 50-53). Peritonitis in rodents, such as mice, can provide essential data on the importance of targets. The end point may be lethality or clinical signs can be monitored. Variation in infection dose in comparison to outcome allows evaluation of the virulence of individual strains.

A variety of other in vivo models are available and may be used when appropriate for specific pathogens or specific test agents. For example, target organ recovery assays (Gordee et al., 1984, J. Antibiotics 37:1054-1065; Bannatyne et al., 1992, Infect. 20:168-170) may be useful for fungi and for bacterial pathogens which are not acutely virulent to animals.

It is also relevant to note that the species of animal used for an infection model, and the specific genetic make-up of that animal, may contribute to the effective evaluation of the effects of a particular test agent. For example, immuno-incompetent animals may, in some instances, be preferable to immuno-competent animals. For example, the action of a competent immune system may, to some degree, mask the effects of the test agent as compared to a similar infection in an immuno-incompetent animal. In addition, many opportunistic infections, in fact, occur in immuno-compromised patients, so modeling an infection in a similar immunological environment is appropriate.

7. Pharmaceutical Compositions

Pharmaceutical compositions of this invention include, for example, those compounds that bind to a protein or other molecule of interest, or a pharmaceutically acceptable salt thereof, and a pharmaceutically acceptable carrier, adjuvant, or vehicle.

The term “pharmaceutically acceptable carrier” refers to a carrier(s) that is “acceptable” in the sense of being compatible with the other ingredients of a composition and not deleterious to the recipient thereof. Optionally, the pH of the formulation is adjusted with pharmaceutically acceptable acids, bases, or buffers to enhance the stability of the formulated compound or its delivery form.

Methods of making and using such pharmaceutical compositions are also included in the invention. The pharmaceutical compositions of the invention can be administered orally, parenterally, by inhalation spray, topically, rectally, nasally, buccally, vaginally, or via an implanted reservoir. Oral administration or administration by injection is preferred. The term parenteral as used herein includes subcutaneous, intracutaneous, intravenous, intramuscular, intra-articular, intrasynovial, intrasternal, intrathecal, intralesional, and intracranial injection or infusion techniques.

Dosage levels of between about 0.01 and about 100 mg/kg body weight per day, preferably between about 0.5 and about 75 mg/kg body weight per day of the subject compounds described herein are useful for the prevention and treatment of various diseases and conditions. In certain cases, the pharmaceutical compositions of this invention will be administered from about 1 to about 5 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

Exemplification

The invention now being generally described, it will be more readily understood by reference to the following examples which are included merely for purposes of illustration of certain aspects and embodiments of the present invention, and are not intended to limit the invention in any way.

### EXAMPLE 1

### Method for Expressing Selmet Labeled Polypeptides

Cells are transformed with a plasmid harboring the gene of interest and inoculated into 20 ml of NMM (New Minimal Medium) and shaken at 37° C. for 8-9 hours. This culture is then transferred into a 6L Erlenmeyer flask containing 2L of minimum medium (M9). The media is supplemented with all amino acids except methionine. All amino acids are added as a solution except for Tyrosine, Tryptophan and Phenylalanine which are added to the media in powder format. As well the media is supplemented with MgSO₄ (2 mM final concentrtion), FeSO₄.7H₂O (25 mg/L final concentration), Glucose (0.4% final concentration), CaCl₂ (0.1 mM final concentration) and Seleno-L-Methionine (40 mg/L final concentration). When the OD₆₀₀ of the cell culture reaches 0.8-0.9, IPTG (0.4 mM final concentration) is added to the medium for protein induction, and the cell culture is kept shaking at 15° C. for 10 hours. The cells are harvested by centrifuged at 3500 rpm at 4° C. for 20 minutes and the cell pellet is resuspended in 15 mL cold binding buffer (Hepes 50 mM, pH 7.5) and 100 μl of protease inhibitors (PMSF and Benzamidine) and flash frozen. The protein is then purified as described below.

### EXAMPLE 2

### Expression of ¹⁵N Labeled Polypeptides

Cells are transformed with a plasmid harboring the gene of interest and inoculated into 2L of minimal media (containing ¹⁵N isotope, Cambridge Isotope Lab) in a 6L Erlenmeyer flask. The minimal media is supplemented with 0.01 mM ZnSO₄, 0.1 mM CaCl₂, 1 mM MgSO₄, 5 mg/L Thiamine.HCl, and 0.4% glucose. The 2L culture is grown at 37° C. and 200 rpm to an OD₆₀₀ of between 0.7-0.8. The culture is then induced with 0.5 mM IPTG and allowed to shake at 15° C. for 14 hours. The cells are harvested by centrifugation and the cell pellet is resuspended in 15 mL cold binding buffer and 100 μl of protease inhibitor and flash frozen. The protein is then purified as described below.

### EXAMPLE 3

### Purification of Polypeptides

The frozen pellets are thawed and sonicated to lyse the cells (5×30 seconds, output 4 to 5, 80% duty cycle, in a Branson Sonifier, VWR). The lysates are clarified by centrifugation at 14,000 rpm for 60 min at 4° C. to remove insoluble cellular debris. The supernatants are removed and supplemented with 1 μl of Benzonase Nuclease (25 U/μl, Novagen).

The recombinant protein is purified using DE52 (anion exchanger, Whatman) and Ni-NTA columns (Qiagen). The DE52 columns (30 mm wide, Biorad) are prepared by mixing 10 grams of DE52 resin in 25 ml of 2.5 M NaCl per protein sample, applying the resin to the column and equilibrating with 30 ml of binding buffer (50 mM in HEPES, pH 7.5, 5% glycerol (v/v), 0.5 M NaCl, 5 mM imidazole). Ni-NTA columns are prepared by adding 3.5-8 ml of resin to the column (20 mm wide, Biorad) based on the level of expression of the recombinant protein and equilibrating the column with 30 ml of binding buffer. The columns are arranged in tandem so that the protein sample is first passed over the DE52 column and then loads directly onto the Ni-NTA column.

The Ni-NTA columns are washed with at least 150 ml of wash buffer (SOmM HEPES, pH 7.5, 5% glycerol (v/v), 0.5 M NaCl, 30 mM imidazole) per column. A pump set at 3.00 to 12.00 may be used to load and/or wash the columns. The protein is eluted off of the Ni-NTA column using elution buffer (50 mM in HEPES, pH 7.5, 5% glycerol (v/v), 0.5 M NaCl, 250 mM imidazole) until no more protein is observed in the aliquots of eluate as measured using Bradford reagent (Biorad). The eluate is supplemented with 1 mM of EDTA and 0.2 mM DTT.

The samples are assayed by SDS-PAGE and stained with Coomassie Blue, with protein purity determined by visual staining.

Samples of purified polypeptide are supplemented with 2.5 mM CaCl₂ and an appropriate amount of Thrombin (the amount added will vary depending on the activity of the enzyme preparation) and incubated for 20-30 minutes on ice in order to remove the His tag from the recombinant protein. The protein sample is then dialyzed in dialysis buffer (10 mM HEPES, pH 7.5, 5% glycerol (v/v) and 0.5 M NaCl) for at least 8 hours using a Slide-A-Lyzer (Pierce) appropriate for the molecular weight of the recombinant protein. An aliquot of the cleaved and dialyzed samples is then assayed by SDS-PAGE and stained with Coomassie Blue to determine the purity of the protein and the success of Thrombin cleavage.

The remainder of the sample is centrifuged at 2700 rpm at 4° C. for 10-15 minutes to remove any precipitant and supplemented with 100 μl of protease inhibitor cocktail (0.1 M benzamidine and 0.05 M PMSF) (NO Bioshop). The protein is then applied to a second Ni-NTA column (˜8 ml of resin) to remove the His-tags and eluted with binding buffer or wash buffer until no more protein is eluting off the column as assayed using the Bradford reagent. The eluted sample is supplemented with 1 mM EDTA and 0.6 mM of DTT and concentrated to a final volume of ˜15 mls using a Millipore Concentrator with an appropriately sized filter at 2700 rpm at 4° C. The samples are then dialyzed overnight against crystallization buffer and concentrated to final volume of 0.3-0.7 ml.

### EXAMPLE 4

### Sample Preparation for Mass Spectrometry—Limited Proteolysis of Polypeptides

The polypeptide is incubated with four different proteases, trypsin, chymotrypsin, papain and proteinase K (Sigma) that are immobilized on plastic 96-well microtitre plates (Nuclon) in the following manner. The protease stocks are made 0.5 mg/ml in TBS (50 mM Tris pH 8, 150 mM NaCI). A serial dilution of each protease is prepared to final concentrations of 50 μg/ml, 25 μg/ml, 5 μg/ml, 2.5 μg/ml and 0.5 μg/ml in TBS. 50 μl of each dilution is then applied to different wells in a row of the microtitre plate. The plate with the arrayed protease dilutions is then incubated overnight at 4° C. in a sealed bag containing a wet paper towel. The protease solution is then removed and the wells washed with 100 μl of blocking buffer (TBS, 0.01% beta-octyl glucoside). The first wash is discarded and the non-specific binding sites on the microtitre wells are blocked with an additional 30 minute incubation at 4° C. with an additional 100 μl of blocking buffer.

A polypeptide solution is then added to each of the protease-coated wells and incubated for 2-4 hours at room temperature. The protein solution is then brought up to 2% Sodium dodecyl sulphate, 25% glycerol, 0.1M Tris-Hel (pH 8.0) and resolved by gel electrophoresis.

### EXAMPLE 5

### Sample Preparation for Mass Spectrometry—Complete Proteolysis of Polypeptides

Gel slices containing the fragments of the polypeptide are cut into 1 mm cubes and 10 to 20 μl of 1% acetic acid is added. The gel particles are washed with 100-150 μl of HPLC grade water (5 minutes with occasional mixing), briefly centrifuged, and the liquid is removed. Acetonitrile (˜200 μl, approximately 3 to 4 times the volume of the gel particles) is added followed by incubation at room temperature for 10 to 15 minutes with vortexing. A second acetonitrile wash may be required to completely dehydrate the gel particles. The sample is briefly centrifuged and all the liquid is removed.

The protein in the gel particles is reduced at 50 degrees Celsius using 10 mM dithiothreitol (in 100 mM ammonium bicarbonate) for 30 minutes and then alkylated at room temperature in the dark using 55 mM iodoacetamide (in 100 mM ammonium bicarbonate). The gel particles are rinsed with a minimal volume of 100 mM ammonium bicarbonate before a trypsin (50 mM ammonium bicarbonate, 5 mM CaCl₂, and 12.5 ng/μl trypsin) solution is added. The gel particles are left on ice for 30 to 45 minutes (after 20 minutes incubation more trypsin solution is added). The excess trypsin solution is removed and 10 to 15 μl digestion buffer without trypsin is added to ensure the gel particles remain hydrated during digestion. The samples are digested overnight at 37° C.

The following day, the supernatant is removed from the gel particles. The peptides are extracted from the gel particles with 2 changes of 100 μL of 100 mM ammonium bicarbonate with shaking for 45 minutes and pooled with the initial gel supernatant. The extracts are acidified to 1% (v/v) with 100% acetic acid.

### EXAMPLE 6

### Purification of Proteolytic Fragments from Complete or Limited Digestions

The peptides are purified with a C18 reverse phase resin. 250 μL of dry resin is washed twice with methanol and twice with 75% acetonitrile/1% acetic acid. A 5:1 slurry of solvent resin is prepared with 75% acetonitrile/1% acetic acid. To the extracted peptides, 2 μL of the resin slurry is added and the solution is shaken at moderate speed for 30 minutes at room temperature. The supernatant is removed and replaced with 200 μL of 2% acetonitrile/1% acetic acid and shaken for 5-15 minutes with moderate speed. The supernatant is removed and the peptides are eluted from the resin with 15 μL of 75% acetonitrile/1% acetic acid with shaking for about 5 minutes. The peptide and slurry mixture is applied to a filter plate and centrifuged for 1-2 minutes at 1000 rpm, the filtrate is collected and stored at −70° C. until use.

Alternatively, the peptides may be purified using ZipTip_{C18} (Millipore, Cat #ZTC18S960). The ZipTips are first pre-wetted by aspirating and dispensing 100% methanol 5 times. The tips are then washed with 2% acetonitrile/1% acetic acid (5 times), followed by 65% acetonitrile/1% acetic (5 times) and returned to 2% acetonitrile/1% acetic acid (5 times). The ZipTips are replaced in their rack and the residual solvent is eliminated. The ZipTips are washed again with 2% acetonitrile/1% acetic acid (5 times). The digested peptides are bound to the ZipTips by aspirating and dispensing the samples 5 times. Salts are removed by washing ZipTips with 2% acetonitrile/1% acetic acid (5 times). 10 μL of 65% acetonitrile/1% acetic acid is collected by the ZipTips and dispensed into a 96-well microtitire plate. 1 μL of sample and 1 μL of matrix are spotted on a MALDI-ToF sample plate for analysis.

### EXAMPLE 7

### Mass Spectrometric Analysis

(a) Method One for Analysis of Tryptic Peptides

Analytical samples containing peptides produced by limited or complete proteolytic digestion are subjected to Matrix Assisted Laser Desorption/Ionization Time Of Flight (MALDI-TOF) mass spectrometry. Samples are mixed 1:1 with a matrix of α-cyano-4-hydroxy-trans-cinnamic acid. The sample/matrix mixture is spotted on to the MALDI sample plate with a robot. The sample/matrix mixture is allowed to dry on the plate and is then introduced into the mass spectrometer. Analysis of the peptides in the mass spectrometer is conducted using both delayed extraction mode and an ion reflector to ensure high resolution of the peptides.

Internally-calibrated peptide masses are searched against databases using a correlative mass matching algorithm. Statistical analysis is performed on each protein match to determine its validity. Typical search constraints include error tolerances within 0.1 Da for monoisotopic peptide masses and carboxyamidomethylation of cysteines. Identified proteins are stored automatically in a relational database with software links to SDS-PAGE images and ligand sequences.

(b) Method Two for Analysis of Tryptic Peptides

Alternatively, samples containing peptides produced by limited or complete proteolytic digestion are analyzed with an ion trap instrument. The peptide extracts are first dried down to approximately 1 μL of liquid. To this, 0.1% trifluoroacetic acid (TFA) is added to make a total volume of approximately 5 μL. Approximately 1-2 μL of sample are injected onto a capillary column (C8, 150 μm ID, 15 cm long) and run at a flow rate of 800 nL/min. using the following gradient program:

| Time (minutes) | % Solvent A | % Solvent B |
|---|---|---|
| 0 | 95 | 5 |
| 30 | 65 | 35 |
| 40 | 20 | 80 |
| 41 | 95 | 5 |

Where Solvent A is composed of water/0.5% acetic acid and Solvent B is acetonitrile/0.5% acetic acid. The majority of the peptides will elute between the 20-40% acetonitrile gradient. Two types of data from the eluting HPLC peaks are acquired with the ion trap mass spectrometer. In the MS¹ dimension, the mass to charge range for scanning is set at 400-1400this will determine the parent ion spectrum. Secondly, the instrument has MS² capabilities whereby it will acquire fragmentation spectra of any parent ions whose intensities are detected to be greater than a predetermined threshold (Mann and Wilm, 1994). A significant amount of information is collected for each protein sample as both a parent ion spectrum and many daughter ion spectra are generated with this instrumentation.

All resulting mass spectra are submitted to a database search algorithm for protein identification. A correlative mass algorithm is utilized along with a statistical verification of each match to identify a protein's identification (Ducret et al, 1998). This method proves much more robust than MALD-ToF mass spectrometry for identifying the components of complex mixtures of proteins. See Mann M, Wilm M, Error-tolerant identification of peptides in sequence databases by peptide sequence tags, Anal Chem 1994 Dec 15;66(24):4390-4399; and Ducret A, Van Oostveen I, Eng J K, Yates J R 3rd, Aebersold R, High throughput protein characterization by automated reverse-phase chromatography/electrospray tandem mass spectrometry, Protein Sci 1998 Mar;7(3):706-719.

### EXAMPLE 8

### NMR Screening

Purified protein sample is centrifuged at 13,000 rpm for 10 minutes with a bench-top microcentrifuge to eliminate any precipitated protein. The supernatant is then transferred into a clean tube and the sample volume is measured. If the sample volume is less than 450 μl, an appropriate amount of crystal buffer is added to the sample to reach that volume. Then 50 μl of D₂O (99.9%) is added to the sample to make an NMR sample of 500 μl.

NMR screening experiments are performed on a Varian Unity 500 spectrometer. All spectra are recorded at 25° C. Standard 1D proton pulse sequence with presaturation is used for iD screening. Normally, a sweepwidth of 6400 Hz, and 32 or 64 scans is used, although different pulse sequences are known to those of skill in the art and may be readily determined. For ¹H, ¹⁵N HSQC experiments, a pulse sequence with “flip-back” water suppression may be used. Typically, sweepwidths of 8000 Hz and 2000 Hz are used for F2 and F1 dimension, respectively. Eight to sixteen scans are normally adequate for a good NMR sample. The data is then processed on a Sun Ultra 5 computer with NMRpipe software.

### EXAMPLE 9

### X-ray Crystallography

(a) Crystallization

Subsequent to purification, a polypeptide is centrifuged for 10 minutes at 4° C. and at 14,000 rpm in order to sediment any aggregated protein. The protein sample is then diluted in order to provide multiple concentrations for screening.

Two 96 well plates (Nunc) are employed for the initial crystal screen, with 48 potential crystallization conditions. The screening library has crystallization conditions found in Hampton Research Crystal Screen I (Jankarik, J. and S. H. Kim, J. Appl. Cryst., 1991. 24:409-11), Hampton Research Crystal Screen II, Hampton Crystal Screen I-Lite, and from Emerald Biostructures, Inc., Bainbridge Island, Wash., Wizard I, Wizard II, Cryo I and Cryo II. Alternatively, other conditions known to those of skill in the art, including those provided in screening kits available from other companies, may also be tested.

Conditions are tested at both protein concentrations and at two temperatures (4 and 20° C.). Crystal setups may be performed by a liquid handling robot appropriately programmed for sitting drop experiments. The robot loads 50 μl of buffer into each screening well on a 24 or 96 well sitting drop crystal screen tray, and then loads 0.5-5 μl of protein into each drop reservoir to be screened on the plate. Subsequently, the robot loads 1.5 μl of the corresponding screening solution into the drop reservoir atop the protein. The plate is then sealed using transparent tape, and stored at either 4, 20 or 35° C. Each plate is observed two days, two weeks, and 1 month after being set. Alternatively, screens may be performed using 0.1-10 μl drops suspended at the interface of two immiscible oils. The protein containing solution has a density intermediate between the two oils and thus floats between them (Chayen N. E.: 1996, Protein Eng. 9:927-29). This procedure may be performed in an automated fashion by an appropriately programmed liquid handling robot, with additional steps being required initially to introduce the oils. No tape is added to facilitate gradual drying out of the drop to promote crystallization.

Having identified conditions that are best suited for further crystal refinement, subsequent plates are set up to explore the effects of variables such as temperature, pH, salt or PEG concentration on crystal size and form, with the intent of establishing conditions where the protein is able to form crystals of suitable size and morphology for diffraction analysis. Each refinement is performed in the sitting drop format in a 24 well lindbro plate. Each well in the tray contains 500 μl of screening solution, and a 1.5 μl drop of protein diluted with 1.5 μl of the screening solution is set to hang from the siliconized glass cover slip covering the well. Alternatively, refinement steps may be performed using either the machine 96 well plate hanging drop method or the oil suspension method described above.

(b) Heavy Atom Substitution

For preparation of crystals containing heavy atoms, crystals of the subject polypeptide may be soaked in a solution of a compound containing the appropriate heavy atom for such period as time as may be experimentally determined is necessary to obtain a useful heavy atom derivative for x-ray purposes. Likewise, for other compounds that may be of interest, including, for example, inhibitors or other molecules that interact with the subject polypeptide, crystals of the subject polypeptide may be soaked in a solution of such compound for an appropriate period of time.

(c) Data Collection and Processing

Before data collection may commence, a protein crystal is frozen to protect it from radiation damage. This is accomplished by suspending the crystal in a loop purchased from Hampton Research) in a stream of dry nitrogen gas at approximately 100 K. The crystals are protected from damage caused by formation of ice crystals (within the lattice or in the liquid surrounding the crystal) upon freezing by supplementing the crystal growth solution with the appropriate cryo-protecting chemical. In some instances, crystals will grow in conditions that provide good cryo-protection, allowing the crystals to be frozen without further modification. In other instances, cryo-protection is achieved by supplementing the crystal growth solution with one or more of the following: 30% volume/volume MPD; 1.2M Na citrate; 30% PEG 400; 4.0M Na Formate; 15% glycerol; 15% ethylene glycol. Alternatively, data may be collected from crystals placed in a thin walled glass capillary and sealed at both ends to protect the crystal from dehydration.

In some cases, data collection is done at the Com-CAT beam-line at the Advanced Photon Source, using a charged coupled device detector. The oscillation method is used. Data is collected for three different wavelengths corresponding to the maximum of anomalous scattering for the appropriate heavy atom, such as selenium, the inflection point and a high energy remote wavelength. Alternatively, data may be collected at only one wavelength corresponding to the maximum of anomalous scattering, with data being collected over a larger range of oscillation angles.

In other cases, data collection is performed in house using a Bruker AXS Proteum R diffractometer. This machine includes a copper rotating anode, Osmic confocal focusing optics and a charge coupled device detector. This data is collected using Cu K_{α} radiation with a wavelength of 1.54 Å, using the oscillation method.

In some instances, data processing is done using the program HKL2000 and data scaling in Scalepack (Z. Otwinowski and W. Minor, Methods in Enzymology vol. 276 p307-326, Academic press). Or, as an alternative, data processing is done using the program Mosfilm and scaling in Scala (Diederichs, K. & Karplus, P. A., Nature Structural Biology, 4, 269-275, 1997).

After scaling, a computer file is obtained which contains the space group, unit cell parameters, and the index, intensity and sigma value for each reflection unique symmetrically. This information forms the raw input of structure detennination.

(d) Heavy Atom Substructure, Phasing

Anomalous scattering sites are found using automated anomalous difference Patterson methods in the program CNX (Brünger A T, Adams P D, Clore G M, DeLano W L, Gros P, Grosse-Kunstleve R W, Jiang J S, Kuszewski J, Nilges M, Pannu N S, Read R J, Rice L M, Simonson T, Warren G L. Acta Crystallogr. D 1998 54 pp 905-21). Alternatively, anomalous scattering sites are found using by real/reciprocal space cycling searches as implemented in shake-and-bake (Weeks C M, DeTitta G T, Hauptman H A, Thuman P, Miller R Acta Crystallogr A 1994; V50: 210-20).

Heavy atom substructure refinement, phase calculation and map calculation are performed in CNX (Brünger A T, et. al. Acta Crystallogr. D 1998 54 pp 905-21), as are density modification (including solvent flipping and non-crystallographic symmetry averaging). In some instances density modification is performed in programs of the CCP4 suite including DM (Collaborative Computational Project, Number 4. 1994. Acta Cryst. D50, 760-763).

The initial protein model may be built in the program TURBO or O. In this process, the crystallographer displays the electron density map on a graphics terminal and interprets the observed density in terms of amino acid residues in the appropriate sequence. Alternatively, QUANTA may be used, which provides an environment for semi-automated model building (Oldfield, T J. Acta Crystallogr D 2001; 57:82-94).

In certain circumstances, the electron density is fully and automatically interpreted in terms of a polypeptide chain using MAID (Levitt, D. G., Acta Crystallogr D 2001 V57:1013-9) or wARP (Perrakis, A., Morris, M. & Lamzin, V. S.; Nature Structural Biology, 1999 V6: 458-463).

(e) Molecular Replacement

In cases where an atomic model sufficiently similar to the structure in question is available, structure solution may proceed by molecular replacement (Rossmann M. G., Acta Crystallogr. A 1990; V46: 73-82). An appropriate search model is identified on the basis of sequence similarity to a suitable target molecule for which a known structure exists in the RCSB protein structure database (http://www.rcsb.org/pdb) or some other (potentially proprietary) database. Alternatively, the molecular replacement solution may be found using genetic algorithms that simultaneously search rotation and translation space, as is done by EPMR (Kissinger C R, Gehlhaar D K, Fogel D B. Acta Crystallogr D 1999; 55: 484-491). The appropriately positioned model may then be refined using rigid body refinement techniques in CNX. This model is then used to calculate model phases, which after solvent flipping in CNX, is used to calculate a map. This map is then used to rebuild the model to better reflect the electron density.

(f) Structure Refinement

The atomic model built by the crystallographer may be used, via theoretical models of how atoms scatter x-rays, to predict the diffraction intensities such a molecule would produce. These predictions can then be compared to the experimentally observed data, allowing the calculation of goodness of fit statistics such as the R-factor. Another important statistic is the R-free, a cross-correlated R-factor calculated using data that has been excluded from model refinement from the beginning. This statistic is free of model bias and can be used, for example, as an objective judge as whether the introduction of extra degrees of freedom into the model is justified (Brunger A T, Clore G M, Gronenborn A M, Saffrich R, Nilges M. Science 1993;261: 328-31). The model was then iteratively perturbed computationally to maximize the probability that the observed data was produced by the model, as well as to optimize model geometry (as embodied in an energy term) in the process known as refinement. Pragmatically, in order to maximize the computational efficiency convergence radius of refinement, simulated annealing refinement using torsion angle dynamics (in order to reduce the degrees of freedom of motion of the model) (Adams P D, Pannu N S, Read R J, Brunger A T, Acta Crystallogr. D 1999; V55: 181-90). Alternatively, refinement may be performed in the CCP4 program REFMAC, which uses similar procedures (Murshudov, G. N., Vagin, A. A. & Dodson, E. J. (1997). Acta Cryst. D53, 240-253).

Experimental phase information from a MAD experiment may be collected and may be utilized as an additional restraint in the refinement as Hendrickson-Lattman phase probability targets. Individual or group temperature factor refinements may also be performed in CNX.

Automatic water picking routines (implemented in the same package) may be employed to find well ordered solvent molecules, the inclusion of which is justified by a reduction in R-free.

Equivalents

The present invention provides among other things methods for determining three dimensional structure information of a polypeptide, methods for identifying compounds that bind to a polypeptide, and methods determining the selectivity of compound for two or more polypeptides. While specific embodiments of the subject invention have been discussed, the above specification is illustrative and not restrictive. Many variations of the invention will become apparent to those skilled in the art upon review of this specification. The appended claims are not intended to claim all such embodiments and variations, and the full scope of the invention should be determined by reference to the claims, along with their full scope of equivalents, and the specification, along with such variations.

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference are the following: WO 00/45168, WO 00/79238, WO 00/77712, EP 1047108, EP 1047107, WO 00/72004, WO 00/73787, WO00/67017, WO 00/48004, WO 00/45168, WO 00/45164, U.S. Ser. No. 09/20,272; PCT/CA99/00640; U.S. Pat. Nos. 6,254,833; 6,232,114; 6,229,603; 6,221,612; 6,214,563; 6,200,762; 6,171,780; 6,143,492; 6,124,128; 6,107,477; D428,157; 6,063,338; 6,004,808; 5,985,214; 5,981,200; 5,928,888; 5,910,287; 6,248,550; 6,232,114; 6,229,603; 6,221,612; 6,214,563; 6,200,762; 6,197,928; 6,180,411; 6,171,780; 6,150,176; 6,140,132; 6,124,128; 6,107,066; 6,077,707; 6,066,476; 6,063,338; 6,054,321; 6,054,271; 6,046,925; 6,031,094; 6,008,378; 5,998,204; 5,981,200; 5,955,604; 5,955,453; 5,948,906; 5,932,474; 5,925,558; 5,912,137; 5,910,287; 5,866,548; 5,834,436; 5,777,079; 5,741,657; 5,693,521; 5,661,035; 5,625,048; 5,602,258; 5,552,555; 5,439,797; 5,374,710; 5,296,703; 5,283,433; 5,141,627; 5,134,232; 5,049,673; 4,806,604; 4,689,432; 4,603,209; 6,217,873; 6,174,530; 6,168,784; 6,271,037; 6,228,654; 6,184,344; 6,040,133; 5,910,437; 5,891,993; 5,854,389; 5,792,664; and 6,248,558.

TABLE 1
Gene Families

| | | Biological function | Illustrative | | |
|---|---|---|---|---|---|
| Family | Target | or Therapeutic use | Genes | Accession | References |
| Nuclear | | | | | Aranda A, et al |
| receptors | | | | | Physiol Rev. |
| | | | | | (2001) |
| | | | | | Jul;81(3):1269-304. |
| | | | | | Whitfield GK, |
| | | | | | et al. J Cell |
| | | | | | Biochem. |
| | | | | | (1999) Suppl |
| | | | | | 32-33:110-22. |
| | androgen receptor | prostate cancer, | AR | M20132 | |
| | (AR) | hormone replacement | | | |
| | | therapy, muscle | | | |
| | | wasting disorders | | | |
| | estrogen receptors | Cancer, hormone | ER | X03635 | von Angerer, |
| | | replacement therapy | ER-beta | NM_001437 | E. “The |
| | | | hERR1 | X51416 | Estrogen |
| | | | hERR2 | X51417 | Receptor as a |
| | | | | | Target for |
| | | | | | Rational Drug |
| | | | | | Design” |
| | | | | | (1995), Landes |
| | | | | | Bioscience |
| | | | | | 159 pp |
| | retinoic acid | acne, psoriasis, | RAT | X06538 | Zouboulis, |
| | receptors (RATs) | premalignant skin | RAT-gamma | M57707 | CC. J Eur |
| | | lesions, cancer | RAT-like protein | X52773 | Acad |
| | | | | | Dermatol |
| | | | | | Venereol. |
| | | | | | (2001)15 |
| | | | | | Suppl 3:63-7 |
| | | | | | Nagpal S, |
| | | | | | Chandraratna |
| | | | | | RA. Curr |
| | | | | | Pharm Des. |
| | | | | | (2000) |
| | | | | | Jun;6(9):919-31. |
| | | | | | Khuri FR, |
| | | | | | Lippman SM. |
| | | | | | Semin Surg |
| | | | | | Oncol. (2000) |
| | | | | | Mar;18(2):100-5 |
| | | | | | Chandraratna |
| | | | | | RA. Cutis. |
| | | | | | (1998) |
| | | | | | Feb;61(2 |
| | | | | | Suppl):40-5. |
| | retinoid-X- | acne, psoriasis, | RXR | NM_005123 | Nagpal S, |
| | receptor (RXR) | premalignant skin | RXR-beta | M84820 | Chandraratna |
| | | lesions, diabetes | RXR-gamma | NM_006917 | RA. Curr |
| | | | | | Pharm Des. |
| | | | | | (2000) |
| | | | | | Jun;6(9):919-31 |
| | vitamin-D | Bone disorders | VDR | J03258 | Jones, D. et al. |
| | receptor (VDR) | (osteoporosis, rickets), | | | Physiol Rev |
| | | alopecia, immune | | | (1998) |
| | | disorders, female | | | Oct;78(4):119 |
| | | reproductive | | | 3-231 |
| | | disorders, pancreatic | | | Hewison, M et |
| | | disorders | | | al Baillieres |
| | | | | | Clin |
| | | | | | Endocrinol |
| | | | | | Metab (1994) |
| | | | | | Apr;8(2):305-15 |
| | peroxisome- | well-known | PPAR-alpha | L02932 | Kersten, S., et |
| | proliferator- | target for lipid- | PPAR-beta | L07592 | al EXS (2000) |
| | activated | lowering and | PPAR-gamma | L40904 | 89:141-51 |
| | receptors | antidiabetic drugs, | | | Everett, L. et |
| | | liver disfunction | | | al Liver (2000) |
| | | | | | Jun;20(3): 191-9 |
| | glucocorticoid | chronic inflammatory | Alpha-GR | X03225 | Barnes PJ.Clin |
| | receptors | diseases, | | | Sci (Lond). |
| | | immunosuppression, | | | (1998) |
| | | depression. type 2 | | | Jun;94(6):557- |
| | | diabetes | | | 72. |
| | | | | | Buttgereit F. |
| | | | | | Z Rheumatol. |
| | | | | | (2000) 59 |
| | | | | | Suppl |
| | | | | | 2:II/119-23. |
| | | | | | Steckler T et |
| | | | | | al. Baillieres |
| | | | | | Best Pract Res |
| | | | | | Clin |
| | | | | | Endocrinol |
| | | | | | Metab. (1999) |
| | | | | | Dec; 13(4):597- |
| | | | | | 614. |
| | thyroid hormone | developmental | TR | X04707 | Brent GA. N |
| | receptor (TR) | disorders, thyroid | TR-alpha | M24748 | Engl J Med. |
| | | disorders, cardiac | TR-beta | NM_000476 | 1994 Se |
| | | function, lipid | | 1 | 29;331(13):84 |
| | | metabolism, pituitary | | | 7-53. |
| | | hormone secretion, | | | |
| | | neural development, | | | |
| | | metabolic disorders, | | | |
| | | skin disorders, | | | |
| | | glaucoma | | | |
| | orphan receptors | anxiety, memory, | BD73 | L31785 | Fujisawa Y,et |
| | | narcolepsy, | RORalpha1 | U04897 | al Nippon |
| | | hypertension, obesity | ROR-beta | Y08639 | Rinsho. (2002) |
| | | | ROR gamma | U16997 | Jan;60(l):31- |
| | | | mCAR1 | AF009327 | 7. |
| | | | TR4 | L27586 | |
| | | | novel OR | Z30425 | |
| | | | TR3 | L13740 | |
| | progesterone | contraception, | | M15716 | Wagner, et al. |
| | receptor (PR) | hormone replacement | | | Proc Natl |
| | | therapy, breast | | | Acad Sci USA |
| | | and prostate cancer, | | | (1996) |
| | | inflammation, | | | Aug |
| | | osteoporosis and | | | 6;93(16):8739- |
| | | endometriosis | | | 44 |
| | liver X receptors | atherosclerosis, | LXR-alpha | U22662 | |
| | (subtype of | enlarged heart, | | | |
| | orphan receptor) | obesity, or Type II | | | |
| | | Diabetes; activated by | | | |
| | | oxysterols and are | | | |
| | | believed to have | | | |
| | | important roles in | | | |
| | | regulation of lipid | | | |
| | | homeostasis as well | | | |
| | | as in the immune | | | |
| | | system | | | |
| | mineralocorticoid | cardiovascular | | M16801 | Delyani JA. |
| | receptor (MR) | diseases such as | | | Kidney Int. |
| | | congestive heart | | | (2000) |
| | | failure and | | | Apr;57(4):140 |
| | | hypertension, edema | | | 8-11. |
| | | | | | Sutanto W, et |
| | | | | | al.Med Res |
| | | | | | Rev. (1991) |
| | | | | | Nov;11(6):617- |
| | | | | | 39. |
| | other nuclear | | Ner-I | U07132 | |
| | receptors | | THRA1, ear1 | M24898 | |
| | | | v-erbA related | X12794 | |
| | | | ear-2 | NM_033013 | |
| | | | NRS 1, grp I, | AF411525 | |
| | | | mem 2 | X76930 | |
| | | | NRS 2, grp e, | Z49826 | |
| | | | mem 1 | M29960 | |
| | | | HNF4 | AF121129 | |
| | | | HNF4-gamma | X12795 | |
| | | | TR2-11 | M64497 | |
| | | | PNR | X16155 | |
| | | | v-erbA related | M62760 | |
| | | | ear-3 | X75918 | |
| | | | ARP-1 | XM_037370 | |
| | | | human COUP-TF | NM_004959 | |
| | | | COUP-TFII | XM_056232 | |
| | | | NOT | NM_021969 | |
| | | | NR4A3 | U93553 | |
| | | | NR4A1 | | |
| | | | NR6A1 | | |
| | | | NR0B2 | | |
| | | | hFTF | | |
| Nuclear | | | | | |
| receptor co- | | | | | |
| activators | | | | | |
| and | | | | | |
| modulators | | | | | |
| | Human steroid | | | U59302 | |
| | receptor | | | | |
| | coactivator-1 F- | | | | |
| | SRC-1 | | | | |
| | SHP | Regulator of estrogen | | L76571 | Johansson, L., |
| | | receptor activity | | | et al J. Biol. |
| | | | | | Chem. (1999) |
| | | | | | 274, 345-353. |
| | DAX-1 | adrenal hypoplasia | | S74720 | Tabarin A.Ann |
| | | congentia; dosage | | | Endocrinol |
| | | sensitive sex reversal | | | (Paris). (2001) |
| | | (DSS) phenotype, | | | Apr;62(2):202- |
| | | a male-to-female sex- | | | 6. |
| | | reversal syndrome due | | | Goodfellow |
| | | to the duplication of a | | | PN, Camerino |
| | | small | | | G. EXS (2001) |
| | | region of human | | | (91):57-69 |
| | | chromosome Xp2.1 | | | |
| Phosphodies | | | | | Perry MJ, |
| terases | | | | | Higgs GA. |
| | | | | | Curr Opin |
| | | | | | Chem Biol |
| | | | | | (1998) |
| | | | | | Aug;2(4):472-81 |
| | PDE-I | CNS, vasorelaxation, | PDE1A | XM_046310 | |
| | | type 2 diabetes | PDE1B | XM_028708 | |
| | | | PDE1C | NM_005020 | |
| | PDE-II | | PDE2A | NM_002599 | |
| | PDE-III | vascular and airway | PDE3A | NM_000921 | |
| | | dilation, platelet | PDE3B | XM_006210 | |
| | | aggregation, obesity | | | |
| | | cytokine production | | | |
| | | and lipolysis | | | |
| | PDE-IV | control of airway | PDE4A | U68532 | Barnette MS, |
| | | smooth muscle and | PDE4B | NM_002600 | Underwood |
| | | inflammatory | PDE4C | NM_000923 | DC. Curr Opin |
| | | mediator release but | PDE4D | XM_041704 | Pulm Med |
| | | also | | | (2000) |
| | | has a role in CNS and | | | Mar;6(2): 164- |
| | | in regulation of gastric | | | 9 |
| | | acid secretion, COPD, | | | |
| | | memory | | | |
| | PDE-V | platelet aggregation, | PDE5A | NM_033437 | |
| | | impotence | | | |
| | PDE-VI | photoreceptors | PDE6A | XM_003786 | |
| | | | PDE6B | XM_018542 | |
| | | | PDE6C | NM_006204 | |
| | | | PDE6D | XM_002246 | |
| | | | PDE6G | NM_002602 | |
| | | | PDE6H | NM_006205 | |
| | PDE-VII | | PDE7A | XM_037534 | |
| | | | PDE7B | NM_018945 | |
| | PDE-VIII | | PDE8A | XM_031443 | |
| | | | PDE8B | XM_041695 | |
| | PDE-IX | | PDE9A | XM_032992 | |
| | PDE-X | | PDE10A | XM_033069 | |
| | PDE-XI | CNS, reproduction | PDE11A | NM_016953 | Fawcett, L. et |
| | | | | | a Proc. Natl. |
| | | | | | Acad. USA |
| | | | | | (2002) |
| | | | | | 97:3702-7.1 |
| Kinases | | | | | Griffin J |
| | | | | | Semin Oncol. |
| | | | | | (2001) |
| | | | | | Oct;28(5 |
| | | | | | Suppl 17):3-8. |
| | MAP (mitogen | cancer, immune | p38 MAP kinase | L35253 | Sebolt- |
| | activated protein) | disorders | MAPK12 | NM_002969 | Leopold JS. |
| | kinases and MAP | | MEKK1 | AF042838 | Oncogene. |
| | kinase kinases | | MAPKAP2 | NM_004759 | (2000) Dec |
| | | | MAPKAP3 | NM_004635 | 27;19(56):659 |
| | | | STE20-like | NM_006281 | 4-9. |
| | | | kinases | NM_003188 | |
| | | | MAP3K7 | U35002 | |
| | | | JNK | | |
| | Tyrosine kinases | cancer, immune | FAK | AF025651 | Traxler P, et |
| | | disorders, anti- | Ab1 | NM_005157 | al. Med Res |
| | | inflammatory | EphA2 | NM_004431 | Rev. (2001) |
| | | | src kinase | M14676 | Nov;21(6):499- |
| | | | Brk/Slk and other | U00803 | 512. |
| | | | src-like kinases | XM_044659 | |
| | | | FRK | NM_004327 | |
| | | | Csk | Z29630 | |
| | | | Bcr | U00803 | |
| | | | ZAP-70/Syk | XM_044444 | |
| | | | Janus JAK | U33284 | |
| | | | Tec | D37827 | |
| | | | FAK/PYK2 | | |
| | | | ERK | | |
| | Serine/Threonine | | TGFbeta | AB009358 | |
| | kinases other than | | activated kin | U01337 | |
| | MAP kinases | | Raf | M80335 | |
| | | | Protein Kinase A | X61037 | |
| | | | Protein Kinase B | XM_034737 | |
| | | | Protein Kinase C | AB013385 | |
| | | | NIK | AF012890 | |
| | | | IKK | XM_041406 | |
| | | | TTK/Esk | AJ010119 | |
| | | | Rsk | M60724 | |
| | | | p70 56 | | |
| | Histidine Kinases | target for | | | Matsushita M, |
| | (present in plants, | antimicrobial agents | | | et al. Bioorg |
| | bacteria, fungi- | | | | Med Chem. |
| | not all are | | | | (2002) |
| | soluble; should | | | | Apr;10(4):855- |
| | we include?) | | | | 67.\ |
| | Polo-Like Kinase | cancer, Alzheimer's | Weel Hu | X62048 | |
| | | | Mytl | NM_004535 | |
| | Mos Proto- | proto-oncogene | | M19412 | |
| | Oncogene | | | | |
| | PRK/TIK | proto-oncogene | | M65029 | |
| | integrin-linked | cancer | ILK-1 | AF244139 | |
| | kinases | | | | |
| | G-protein coupled | | GRK-7 | AF282269 | |
| | receptor kinases | | | | |
| | (GRKs) | | | | |
| | Hexokinase | cancer | type II | NM_000189 | |
| | Casein Kinase | | | | |
| | Glycogen | | | | |
| | Synthase Kinase | | | | |
| | (GSK), | | | | |
| | LIM Kinase | | | | |
| | (actin-binding | | | | |
| | kinase | | | | |
| | IkappaB | | | | |
| | Kinases (IKK), | | | | |
| | Rock and Related | | | | |
| | Rho Interacting | | | | |
| | Proteins (protein | | | | |
| | kinases) | | | | |
| | Pyruvate | | | | |
| | Dehydrogenase | | | | |
| | Kinase (PDK) | | | | |
| | IL-1 receptor | | | | |
| | kinases | | | | |
| | Calcium/calmodu | | | | |
| | lin-dependent | | | | |
| | protein kinases | | | | |
| | (CaM kinase) | | | | |
| | PAK and Related | | | | |
| | “CRIB” Domain | | | | |
| | Protein Kinases | | | | |
| | Muscle-Specific | | | | |
| | Kinase (MuSK) | | | | |
| Phosphatass | protein tyrosine | diabetes, obesity, and | PTP1b | AY029236 | Zhang ZY. |
| | phosphatase | impaired glucose | SHP-1 | XM_092897 | Curr |
| | (PTP) | tolerance | SHP-2 | XM_069073 | Opin Chem |
| | | | | | Biol. (2001) |
| | | | | | Aug;5(4):416- |
| | | | | | 23. |
| | CDC25 | cancer (key regulator | CDC25A | NM_001789 | Lazo, JS, et |
| | phosphatase | of cell cycle | CDC25B | X96436 | al.. J Med |
| | | progression) | | | Chem. (2001) |
| | | | | | Nov |
| | | | | | 22;44(24):404 |
| | | | | | 2-9. |
| | Ca2+/calmodulin- | immunosuppression | calcineurin A- | NM_000944 | |
| | dependent S/T | (key proteins in | alpha | | |
| | protein | calcium signaling | calcineurin A- | NM_021132 | |
| | phosphatases | pathway) | beta | | |
| | | | calcineurin A- | NM_005605 | |
| | | | gamma | | |
| | | | calcineurin cata. | S46622 | |
| | | | sub. | | |
| | Inositol | Lowe syndrome, | phosphatidylinosi | XM_038489 | Stolz et al, |
| | phosphatases | cancer and | tol | | Genetics |
| | | myotubular myopathy | 4,5-bisphosphate | | (1998) |
| | | | (PIP2) | | 148:1715-1729 |
| | | | inositol | AF200432 | Stolz et al., |
| | | | monophosphatase | | JBC (1998) |
| | | | | | 273:11852-61 |
| | | | inositol | NM_005541 | Raucher et al., |
| | | | polyphosphate 5- | | Cell, (2000) |
| | | | phosphatase | | 100:221-8 |
| | | | inositol | NM_016532, | |
| | | | polyphosphate 4- | NM_130766 | |
| | | | phosphatases | | |
| | | | (4ptases) | | |
| | MAPK | cancer | MAPK Phe-7 | XM_039106 | |
| | phosphatases | | | | |
| | Dual-specificity | cancer | FYVE-DSP1a | AF233436 | |
| | phosphatases | | FYVE-DSP1b | AF233437 | |
| | | | FYVE-DSP1c | AF233438 | |
| | | | FYVE-DSP2 | AF264717 | |

We claim:

1. A method for identifying a compound that binds to a protein, comprising:
(a) providing a purified protein;
(b) subjecting the protein to mass spectroscopy analysis to identify the protein;
(c) subjecting the protein to two or more of the following in any order:
(i) NMR spectroscopic analysis in the absence of the compound;
(ii) NMR spectroscopic analysis in the presence of the compound;
(iii) X-ray diffraction analysis in the absence of the compound; and
(iv) X-ray diffraction analysis in the presence of the compound; and
(c) analyzing the results obtained in (a) and (b) to thereby identify a compound that binds to the protein.

2. The method of claim 1, wherein (c) of the method comprises (i) and (ii).

3. The method of claim 1, wherein (c) of the method comprises (iii) and (iv).

4. The method of claim 1, wherein (c) of the method comprises (ii) and (iii).

5. The method of claim 1, wherein the protein is from a microbial species.

6. The method of claim 1, wherein the protein is from a mammalian species.

7. The method of claim 1, wherein the protein is one of the following: kinases, proteases, phosphatases, P450s, conjugation enzymes, ATPases, GTPase, nucleotide binding proteins, DNA processing enzymes, helicases, polymerases, RNA polymerases, DNA polymerases, GPCRs, intracellular receptors, metabolic enzymes, nuclear receptors, channels, phosphodiesterases, Ca binding proteins, bacterial proteins, non-membrane bacterial proteins, human proteins that bind viral proteins, viral proteins, or nonmembrane viral proteins.

8. A method for identifying a compound that binds to a protein, comprising:
(a) providing a purified protein;
(b) subjecting the protein to three or more of the following in any order:
(i) NMR spectroscopic analysis in the absence of the compound;
(ii) NMR spectroscopic analysis in the presence of the compound;
(iii) X-ray diffraction analysis in the absence of the compound;
(iv) X-ray diffraction analysis in the presence of the compound; and
(v) analysis by mass spectroscopy; and
(c) analyzing the results obtained in (b) to thereby identify a compound that binds to the protein.

9. The method of claim 8, further comprising identifying a compound that binds to the protein using structure guided drug design.

10. The method of claim 9, the structure guided drug design comprising:
(i) supplying a computer modeling application with a set of structure coordinates and structural information obtained from (b);
(ii) supplying the computer modeling application with a set of structure coordinates for a chemical entity; and
(iii) determining whether the chemical entity is expected to bind to the protein.

11. The method of claim 10, wherein (iii) for the structure guided drug design further comprises performing a fitting operation between the chemical entity and a site of the protein, followed by computationally analyzing the results of the fitting operation to quantify the association between the chemical entity and the site.

12. The method of claim 9, wherein the structure guided drug design comprises:
(1) supplying a computer modeling application with a set of structure coordinates and structural information for the protein obtained from (b);
(2) supplying the computer modeling application with a set of structure coordinates for a chemical entity;
(3) evaluating the potential binding interactions between the chemical entity and a site of interest of the protein;
(4) structurally modifying the chemical entity to yield a set of structure coordinates for a modified chemical entity; and
(5) determining whether the chemical entity is expected to bind to the site.

13. The method of claim 9, wherein the structure guided drug design comprises:
(1) supplying a computer modeling application with a set of structure coordinates and structural information for the protein obtained from (b);
(2) computationally building a chemical entity represented by a set of structure coordinates; and
(3) determining whether the chemical entity is expected to bind to the protein.

14. The method of claim 8, which further comprises characterizing the ability of the compound to interact with the three dimensional structure of the protein using a computational method.

15. The method of claim 8, wherein (b) of the method comprises (i), (ii) and (v).

16. The method of claim 8, wherein (b) of the method comprises (iii), (iv) and (v).

17. The method of claim 8, wherein (b) of the method comprises (i), (ii), (iii), (iv) and (v).

18. The method of claim 8, wherein the protein is from a microbial species.

19. The method of claim 8, wherein the protein is from a mammalian species.

20. The method of claim 8, wherein the protein is one of the following: kinases, proteases, phosphatases, P450s, conjugation enzymes, ATPases, GTPase, nucleotide binding proteins, DNA processing enzymes, helicases, polymerases, RNA polymerases, DNA polymerases, GPCRs, intracellular receptors, metabolic enzymes, nuclear receptors, channels, phosphodiesterases, Ca binding proteins, bacterial proteins, non-membrane bacterial proteins, human proteins that bind viral proteins, viral proteins, or nonmembrane viral proteins.

21. The method of claim 8, wherein the method comprises analysis of the protein by mass spectrometry, which further comprises subjecting the protein to proteolytic digestion prior to analysis by mass spectrometry.

22. The method of claim 21, wherein proteolytic digestion of the protein is carried out in the presence of the compound.

23. The method of claim 8, which further comprises determining the ability of the compound to bind to the protein using an activity assay, wherein a change in the activity of the protein in the presence of the compound indicates that the compound binds to the protein.

24. The method of claim 8, wherein the compound is a polypeptide, nucleic acid, or small molecule.

25. The method of claim 24, wherein the compound is a member of a library of compounds.

26. The method of claim 24, wherein the compound is isolated from a naturally occurring source.

27. The method of claim 8, which further comprises identifying a site on the protein, wherein the site region is a location on the three dimensional structure of the protein comprising three or more amino acid residues of the protein.

28. The method of claim 27, wherein the site is identified by comparing structural information of the protein obtained in the presence and absence of the compound.

29. The method of claim 8, wherein the method comprises analysis of the protein by mass spectrometry, and wherein the protein is identified using mass spectrometry to determine the primary sequence of the protein, the type and location of post translational modifications of the protein, or to identify regions of the protein which interact with another molecule.

30. The method of claim 8, wherein the method comprises analysis of the protein by one of the two NMR analyses, and wherein the NMR spectroscopic analysis of the protein is used to determine information about the three dimensional structure, the conformational state, the aggregation level, or the state of unfolding of the protein.

31. The method of claim 8, wherein the method comprises analysis of the protein by one of the two NMR analyses, and wherein the NMR spectroscopic analysis involves iD NMR, 2D NMR or ¹⁵N/¹H correlation spectroscopy.

32. The method of claim 8, wherein the method comprises analysis of the protein by one of the two X-ray diffraction analyses, and wherein the X-ray diffraction analysis is used to determine the three dimensional structure of the stable domain or the space group of crystals of the stable domain.

33. The method of claim 8, wherein the method comprises analysis of the protein by one of the two X-ray diffraction analyses, and further comprising identifying the site of the protein at which the compound binds.

34. The method of claim 33, further comprising conducting structure guided drug design using sets of points with a root mean square deviation of not more than 1.5 Å for all backbone atoms of the amino acid residues of the site.

35. The method of claim 33, further comprising conducting structure guided drug design using sets of points with a root mean square deviation of not more than 1.5 Å for all side chain atoms and Ca atoms of the amino acid residues of the site.

36. The method of claim 8, wherein the protein comprises one or more labels so as to facilitate determining three dimensional structure information of the protein.

37. The method of claim 36, wherein the protein comprises an isotopic label.

38. The method of claim 37, wherein the isotopic label is one of the following: potassium-40 (⁴⁰K), carbon-14 (¹⁴C), tritium (³H), sulphur-35 (³⁵S), phosphorus-32 (³²p), technetium-99m (^{99m}Tc), thallium-201 (²⁰¹Tl), gallium-67 (⁶⁷Ga), indium-111 (¹¹¹In), iodine-123 (123I), iodine-131 (131I), yttrium-90 (⁹⁰Y), samarium-153 (¹⁵³Sm), rhenium-186 (¹⁸⁶Re), rhenium-188 (¹⁸⁸Re), dysprosium-165 (¹⁶⁵Dy), holmium-166 (¹⁶⁶Ho), hydrogen-1 (¹H), hydrogen-2 (²H), hydrogen-3 (³H), phosphorous-31 (³¹p), sodium-23 (²³Na), nitrogen-14 (¹⁴N), nitrogen-15 (¹⁵N), carbon-13 (¹³C) and fluorine-19 (¹⁹F).

39. The method of claim 37, wherein the protein comprises at least two different isotopic labels.

40. The method of claim 39, wherein the protein comprises at least one ¹⁵N label and at least one ¹³C label.

41. The method of claim 36, wherein the protein comprises a heavy atom label.

42. The method of claim 41, wherein the heavy atom label is one of the following: cobalt, selenium, krypton, bromine, strontium, molybdenum, ruthenium, rhodium, palladium, silver, cadmium, tin, iodine, xenon, barium, lanthanum, cerium, praseodymium, neodymium, samarium, europium, gadolinium, terbium, dysprosium, holmium, erbium, thulium, ytterbium, lutetium, tantalum, tungsten, rhenium, osmium, iridium, platinum, gold, mercury, thallium, lead, thorium and uranium.

43. The method of claim 36, wherein the protein comprises at least one seleno-methionine.

44. The method of claim 39, wherein the protein comprises at least one isotopic label and at least one heavy atom label.

45. A computer readable storage medium comprising digitally encoded structural data, wherein the data comprise the identity of a protein, the identity of a compound, and the three dimensional structure information of the protein obtained using the method of claim 8.

46. A database comprising the identity of a protein, the identity of a compound, and the three dimensional structure information of the protein obtained using the method of claim 8.

47. The method of claim 8, wherein the protein is a member of a library of proteins.

48. The method of claim 8, wherein several of the experimental procedures for one or more of the analyses are automated.

49. The method of claim 8, wherein the protein is at least about 80% pure by weight.

50. The method of claim 8, wherein the protein is at least about 70% soluble as measured by light scattering.

51. The method of claim 8, wherein the protein is fused to at least one heterologous polypeptide.

52. The method of claim 8, wherein the crystallized protein diffracts X-rays to a resolution of about 3.5 Å or better.
